SEQUENCE LISTING (148 sequences)

SEQ ID NO: 1 - protein, 18 aa, Severe acute respiratory syndrome-related coronavirus
  source 1..18: note = SARS-CoV-2
MYSFVSEETGTLIVNSVL

SEQ ID NO: 2 - protein, 18 aa, synthetic construct
MYSFVSDDTGTLIVNSVL

SEQ ID NO: 3 - protein, 31 aa, synthetic construct
  REGION 13..14: note = Q is linked to M via 13-atom polyethylene glycol linker; or (PEG)3
GRKKRRQRRRPPQMYSFVSDDTGTLIVNSVL

SEQ ID NO: 4 - protein, 13 aa, Human immunodeficiency virus 1
GRKKRRQRRRPPQ

SEQ ID NO: 5 - protein, 6 aa, synthetic construct
RRRRRR

SEQ ID NO: 6 - protein, 16 aa, Drosophila sp.
RQIKIWFQNRRMKWKK

SEQ ID NO: 7 - protein, 31 aa, synthetic construct
GRKKRRQRRRPPQMYSFVSEETGTLIVNSVL

SEQ ID NO: 8 - protein, 31 aa, synthetic construct; identical to SEQ ID NO: 3

SEQ ID NO: 9 - protein, 24 aa, synthetic construct
RRRRRRMYSFVSEETGTLIVNSVL

SEQ ID NO: 10 - protein, 34 aa, synthetic construct
RQIKIWFQNRRMKWKKMYSFVSEETGTLIVNSVL

SEQ ID NO: 11 - protein, 76 aa, Severe acute respiratory syndrome-related coronavirus
  source 1..76: note = SARS-CoV-1
MYSFVSEETGTLIVNSVLLFLAFVVFLLVTLAILTALRLCAYCCNIVNVSLVKPTVYVYSRVKNLNSSEGVPDLLV

SEQ ID NO: 12 - protein, 75 aa, Severe acute respiratory syndrome-related coronavirus
  source 1..75: note = SARS-CoV-2
MYSFVSEETGTLIVNSVLLFLAFVVFLLVTLAILTALRLCAYCCNIVNVSLVKPSFYVYSRVKNLNSSRVPDLLV

SEQ ID NO: 13 - protein, 22 aa, Severe acute respiratory syndrome-related coronavirus
  source 1..22: note = SARS-CoV-2
MYSFVSEETGTLIVNSVLLFLA

SEQ ID NO: 14 - protein, 18 aa, Middle East respiratory syndrome-related coronavirus
MLPFVQERIGLFIVNFFI

SEQ ID NO: 15 - protein, 18 aa, synthetic construct
MLPFVQDRIGLFIVNFFI

SEQ ID NO: 16 - protein, 18 aa, synthetic construct
MLPFVQEHIGLFIVNFFI

SEQ ID NO: 17 - protein, 18 aa, Human coronavirus 229E
MFLKLVDDHALVVNVLLW

SEQ ID NO: 18 - protein, 18 aa, synthetic construct
MFLKLVEEHALVVNVLLW

SEQ ID NO: 19 - protein, 18 aa, Human coronavirus NL63
MFLRLIDDNGIVLNSILW

SEQ ID NO: 20 - protein, 18 aa, synthetic construct
MFLRLIEENGIVLNSILW

SEQ ID NO: 21 - protein, 18 aa, synthetic construct
MFLRLIEEDGIVLNSILW

SEQ ID NO: 22 - protein, 18 aa, unidentified
  source 1..18: note = HCoV-OC43
MADAYLADTVWYVGQIIF

SEQ ID NO: 23 - protein, 18 aa, synthetic construct
MADAYLAETVWYVGQIIF

SEQ ID NO: 24 - protein, 18 aa, Human coronavirus HKU1
MVDLFFNDTAWYIGQILV

SEQ ID NO: 25 - protein, 17 aa, synthetic construct
VDLFFNETAWYIGQILV

SEQ ID NO: 26 - protein, 82 aa, Middle East respiratory syndrome-related coronavirus
MLPFVQERIGLFIVNFFIFTVVCAITLLVCMAFLTATRLCVQCMTGFNTLLVQPALYLYNTGRSVYVKFQDSKPPLPPDEWV

SEQ ID NO: 27 - protein, 84 aa, unidentified
  source 1..84: note = HCoV-OC43
MFMADAYLADTVWYVGQIIFIVAICLLVTIVVVAFLATFKLCIQLCGMCNTLVLSPSIYVFNRGRQFYEFYNDVKPPVLDVDDV

SEQ ID NO: 28 - protein, 82 aa, Human coronavirus HKU1
MVDLFFNDTAWYIGQILVLVLFCLISLIFVVAFLATIKLCMQLCGFCNFFIISPSAYVYKRGMQLYKSYSEQVIPPTSDYLI

SEQ ID NO: 29 - protein, 77 aa, Human coronavirus 229E
MFLKLVDDHALVVNVLLWCVVLIVILLVCITIIKLIKLCFTCHMFCNRTVYGPIKNVYHIYQSYMHIDPFPKRVIDF

SEQ ID NO: 30 - protein, 77 aa, Human coronavirus NL63
MFLRLIDDNGIVLNSILWLLVMIFFFVLAMTFIKLIQLCFTCHYFFSRTLYQPVYKIFLAYQDYMQIAPVPAEVLNV

SEQ ID NO: 31 - DNA, 28 bp, synthetic construct
  source 1..28: mol_type = other DNA
ctcttgtagatctgttctctaaacgaac

SEQ ID NO: 32 - DNA, 20 bp, synthetic construct
  source 1..20: mol_type = other DNA
ggtccaccaaacgtaatgcg

SEQ ID NO: 33 - DNA, 23 bp, synthetic construct
  source 1..23: mol_type = other DNA
ctcattcgtttcggaagagacag

SEQ ID NO: 34 - DNA, 24 bp, synthetic construct
  source 1..24: mol_type = other DNA
agaccagaagatcaggaactctag

SEQ ID NO: 35 - DNA, 20 bp, synthetic construct
  source 1..20: mol_type = other DNA
cttcaccaccatggagaagg

SEQ ID NO: 36 - DNA, 20 bp, synthetic construct
  source 1..20: mol_type = other DNA
tgaagtcgcaggagacaacc

SEQ ID NO: 37 - DNA, 22 bp, synthetic construct
  source 1..22: mol_type = other DNA
gaaggtgaaggtcggagtcaac

SEQ ID NO: 38 - DNA, 24 bp, synthetic construct
  source 1..24: mol_type = other DNA
tcgttgtcataccaggaaatgagc

SEQ ID NO: 39 - DNA, 20 bp, synthetic construct
  source 1..20: mol_type = other DNA
cttctccgatgcctctgacg

SEQ ID NO: 40 - DNA, 19 bp, synthetic construct
  source 1..19: mol_type = other DNA
cggggcttggaccatacag

SEQ ID NO: 41 - DNA, 19 bp, synthetic construct
  source 1..19: mol_type = other DNA
actcggaccttctcacgtc

SEQ ID NO: 42 - DNA, 20 bp, synthetic construct
  source 1..20: mol_type = other DNA
ggtcggtgtagtggtgatgt

SEQ ID NO: 43 - DNA, 21 bp, synthetic construct
  source 1..21: mol_type = other DNA
ctcaccggatgcaccaatgtt

SEQ ID NO: 44 - DNA, 21 bp, synthetic construct
  source 1..21: mol_type = other DNA
cgcgttgctcacaatgttcat

SEQ ID NO: 45 - protein, 16 aa, synthetic construct
RKKRRRESRKKRRRES

SEQ ID NO: 46 - protein, 17 aa, synthetic construct
GRPRESGKKRKRKRLKP

SEQ ID NO: 47 - protein, 15 aa, synthetic construct
GKRKKKGKLGKKRDP

SEQ ID NO: 48 - protein, 17 aa, synthetic construct
GKRKKKGKLGKKRPRSR

SEQ ID NO: 49 - protein, 18 aa, synthetic construct
RKKRRRESRRARRSPRHL

SEQ ID NO: 50 - protein, 19 aa, synthetic construct
SRRARRSPRESGKKRKRKR

SEQ ID NO: 51 - protein, 19 aa, synthetic construct
VKRGLKLRHVRPRVTRMDV

SEQ ID NO: 52 - protein, 14 aa, synthetic construct
SRRARRSPRHLGSG

SEQ ID NO: 53 - protein, 16 aa, synthetic construct
LRRERQSRLRRERQSR

SEQ ID NO: 54 - protein, 22 aa, synthetic construct
GAYDLRRRERQSRLRRRERQSR

SEQ ID NO: 55 - protein, 9 aa, synthetic construct
RKKRRQRRR

SEQ ID NO: 56 - protein, 15 aa, synthetic construct
RRRRNRTRRNRRRVR

SEQ ID NO: 57 - protein, 17 aa, synthetic construct
TRQARRNRRRRWRERQR

SEQ ID NO: 58 - protein, 13 aa, synthetic construct
TRRQRTRRARRNR

SEQ ID NO: 59 - protein, 19 aa, synthetic construct
KMTRAQRRAAARRNRWTAR

SEQ ID NO: 60 - protein, 17 aa, synthetic construct
NAKTRRHERRRKLAIER

SEQ ID NO: 61 - protein, 22 aa, synthetic construct
MDAQTRRRERRAEKQAQWKAAN

SEQ ID NO: 62 - protein, 18 aa, synthetic construct
TAKTRYKARRAELIAERR

SEQ ID NO: 63 - protein, 16 aa, synthetic construct
TRRNKRNRIQEQLNRK

SEQ ID NO: 64 - protein, 32 aa, synthetic construct
PRRRRSSSRPVRRRRRPRVSRRRRRRGGRRRR

SEQ ID NO: 65 - protein, 28 aa, synthetic construct
RIKAERKRMRNRIAASKSRKRKLERIAR

SEQ ID NO: 66 - protein, 26 aa, synthetic construct
KRRIRRERNKMAAAKSRNRRRELTDT

SEQ ID NO: 67 - protein, 22 aa, synthetic construct
KRARNTEAARRSRARKLQRMKQ

SEQ ID NO: 68 - protein, 16 aa, synthetic construct
RQIKIWFQNRRMKWKK

SEQ ID NO: 69 - protein, 16 aa, synthetic construct
RVIRVWFQNKRCKDKK

SEQ ID NO: 70 - protein, 60 aa, synthetic construct
SKRTRQTYTRYQTLELEKEFHFNRYITRRRRIDIANALSLSERQIKIWFQNRRMKSKKDR

SEQ ID NO: 71 - protein, 16 aa, synthetic construct
SQIKIWFQNKRAKIKK

SEQ ID NO: 72 - protein, 16 aa, synthetic construct
RQVTIWFQNRRVKEKK

SEQ ID NO: 73 - protein, 16 aa, synthetic construct
KQINNWFINQRKRHWK

SEQ ID NO: 74 - protein, 16 aa, synthetic construct
RHIKIWFQNRRMKWKK

SEQ ID NO: 75 - protein, 23 aa, synthetic construct
MGLGLHLLVLAAALQGAKKKRKV

SEQ ID NO: 76 - protein, 30 aa, synthetic construct
MVKSKIGSWILVLFVAMWSDVGLCKKRPKP

SEQ ID NO: 77 - protein, 28 aa, synthetic construct
MANLGYWLLALFVTMWTDVGLCKKRPKP

SEQ ID NO: 78 - protein, 21 aa, synthetic construct
AAVLLPVLLAAPVQRKRQKLP

SEQ ID NO: 79 - protein, 12 aa, synthetic construct
AAVLLPVLLAAP

SEQ ID NO: 80 - protein, 24 aa, synthetic construct
RRIRPRPPRLPRPRPRPLPFPRPG

SEQ ID NO: 81 - protein, 20 aa, synthetic construct
VDKGSYLPRPTPPRPIYNRN

SEQ ID NO: 82 - protein, 22 aa, synthetic construct
KCFQWQRNMRKVRGPPVSCIKR

SEQ ID NO: 83 - protein, 21 aa, synthetic construct
TRSSRAGLQWPVGRVHRLLRK

SEQ ID NO: 84 - protein, 26 aa, synthetic construct
GIGAVLKVLTTGLPALISWIKRKRQQ

SEQ ID NO: 85 - protein, 23 aa, synthetic construct
GIGKWLHSAKKFGKAFVGEIMNS

SEQ ID NO: 86 - protein, 38 aa, synthetic construct
LLGDFFRKSKEKIGKEFKRIVQRIKDFLRNLVPRTESC

SEQ ID NO: 87 - protein, 18 aa, synthetic construct
RGGRLSYSRRRFSTSTGR

SEQ ID NO: 88 - protein, 14 aa, synthetic construct
YKQCHKKGGKKGSG

SEQ ID NO: 89 - protein, 20 aa, synthetic construct
ALWKTLLKKVLKAPKKKRKV

SEQ ID NO: 90 - protein, 20 aa, synthetic construct
HARIKPTFRRLKWKYKGKFW

SEQ ID NO: 91 - protein, 22 aa, synthetic construct
TKRRITPKDVIDVRSVTTEINT

SEQ ID NO: 92 - protein, 27 aa, synthetic construct
RQGAARVTSWLGRQLRIAGKRLEGRSK

SEQ ID NO: 93 - protein, 34 aa, synthetic construct
NAATATRGRSAASRPTQRPRAPARSASRPRRPVQ

SEQ ID NO: 94 - protein, 16 aa, synthetic construct
RHSRIGIIQQRRTRNG

SEQ ID NO: 95 - protein, 29 aa, synthetic construct
KLIKGRTPIKFGKADCDRPPKHSQNGMGK

SEQ ID NO: 96 - protein, 12 aa, synthetic construct
PLSSIFSRIGDP

SEQ ID NO: 97 - protein, 26 aa, synthetic construct
DPKGDPKGVTVTVTVTVTGKGDPKPD

SEQ ID NO: 98 - protein, 10 aa, synthetic construct
RRIPNRRPRR

SEQ ID NO: 99 - protein, 5 aa, synthetic construct
RLRWR

SEQ ID NO: 100 - protein, 22 aa, synthetic construct
MVRRFLVTLRIRRACGPPRVRV

SEQ ID NO: 101 - protein, 22 aa, synthetic construct
MVTVLFRRLRIRRACGPPRVRV

SEQ ID NO: 102 - protein, 18 aa, synthetic construct
LLIILRRRIRKQAHAHSK

SEQ ID NO: 103 - protein, 18 aa, synthetic construct
LSTAADMQGVVTDGMASG

SEQ ID NO: 104 - protein, 28 aa, synthetic construct
LSTAADMQGVVTDGMASGLDKDYLKPDD

SEQ ID NO: 105 - protein, 15 aa, synthetic construct
KFHTFPQTAIGVGAP

SEQ ID NO: 106 - protein, 5 aa, synthetic construct
VPTLK

SEQ ID NO: 107 - protein, 6 aa, synthetic construct
PFVYLI

SEQ ID NO: 108 - protein, 10 aa, synthetic construct
PIEVCMYREP

SEQ ID NO: 109 - protein, 20 aa, synthetic construct
KETWWETWWTEWSQPKKRKV

SEQ ID NO: 110 - protein, 27 aa, synthetic construct
GLAFLGFLGAAGSTMGAWSQPKKKRKV

SEQ ID NO: 111 - protein, 27 aa, synthetic construct
GWTLNSAGYLLGKINLKALAALAKKIL

SEQ ID NO: 112 - protein, 21 aa, synthetic construct
  MOD_RES 17: note = Aib
AGYLLGHINLHHLAHLXHHIL

SEQ ID NO: 113 - protein, 17 aa, synthetic construct
KLALKALKALKAALKLA

SEQ ID NO: 114 - protein, 9 aa, synthetic construct
RRWWRRWRR

SEQ ID NO: 115 - protein, 20 aa, synthetic construct
GLWRALWRLLRSLWRLLWRA

SEQ ID NO: 116 - protein, 23 aa, synthetic construct
LIRLWSHLIHIWFQNRRLKWKKK

SEQ ID NO: 117 - protein, 30 aa, synthetic construct
WEAALAEALAEALAEHLAEALAEALEALAA

SEQ ID NO: 118 - protein, 18 aa, synthetic construct
LKTLTETLKELTKTLTEL

SEQ ID NO: 119 - protein, 18 aa, synthetic construct
QLALQLALQALQAALQLA

SEQ ID NO: 120 - protein, 36 aa, synthetic construct
GPSQPTYPGDDAPVRDLIRFYRDLQRYLNVVTRHRY

SEQ ID NO: 121 - protein, 36 aa, synthetic construct
GPSQPTYPGDDAPVRDLIRFYRDLRRYLNVVTRHRY

SEQ ID NO: 122 - protein, 36 aa, synthetic construct
GPSQPTYPGDDAPVRDLRRFYRDLRRYLNVVTRHRY

SEQ ID NO: 123 - protein, 18 aa, synthetic construct
VRLPPPVRLPPPVRLPPP

SEQ ID NO: 124 - protein, 18 aa, synthetic construct
VELPPPVELPPPVELPPP

SEQ ID NO: 125 - protein, 15 aa, synthetic construct
FKIYDKKVRTRVVKH

SEQ ID NO: 126 - protein, 18 aa, synthetic construct
RASKRDGSWVKKLHRILE

SEQ ID NO: 127 - protein, 16 aa, synthetic construct
KGTYKKKLMRIPLKGT

SEQ ID NO: 128 - protein, 19 aa, synthetic construct
LYKKGPAKKGRPPLRGWFH

SEQ ID NO: 129 - protein, 17 aa, synthetic construct
HSPIIPLGTRFVCHGVT

SEQ ID NO: 130 - protein, 16 aa, synthetic construct
YTAIAWVKAFIRKLRK

SEQ ID NO: 131 - protein, 17 aa, synthetic construct
IAWVKAFIRKLRKGPLG

SEQ ID NO: 132 - protein, 17 aa, synthetic construct
IAWVKAFIRKLRKGPLG

SEQ ID NO: 133 - protein, 14 aa, synthetic construct
RLSGMNEVLSFRWL

SEQ ID NO: 134 - protein, 15 aa, synthetic construct
SDLWEMMMVSLACQY

SEQ ID NO: 135 - protein, 12 aa, synthetic construct
VTWTPQAWFQWV

SEQ ID NO: 136 - protein, 13 aa, synthetic construct
GSPWGLQHHPPRT

SEQ ID NO: 137 - protein, 13 aa, synthetic construct
GPFHFYQFLFPPV

SEQ ID NO: 138 - protein, 12 aa, synthetic construct
TSPLNIHNGQKL

SEQ ID NO: 139 - protein, 9 aa, synthetic construct
CAYHRLRRC

SEQ ID NO: 140 - protein, 18 aa, synthetic construct
RCGRASRCRVRWMRRRRI

SEQ ID NO: 141 - protein, 26 aa, synthetic construct
PYSRPHVQLWYPNRESCRSLIRSLGP

SEQ ID NO: 142 - protein, 12 aa, synthetic construct
PLILLRLLRGQF

SEQ ID NO: 143 - protein, 12 aa, synthetic construct
PLIYLRLLRGQF

SEQ ID NO: 144 - protein, 15 aa, synthetic construct
KLWMRWYSPTTRRYG

SEQ ID NO: 145 - protein, 5 aa, synthetic construct
PMLKE

SEQ ID NO: 146 - protein, 5 aa, synthetic construct
VPALR

SEQ ID NO: 147 - protein, 5 aa, synthetic construct
VSALK

SEQ ID NO: 148 - protein, 5 aa, synthetic construct
IPALK

(19) United States
(12) Patent Application Publication
(10) Pub. No.: US 2025/0026796 A1
(43) Pub. Date: Jan. 23, 2025

(54) INHIBITORY PEPTIDES AGAINST CORONAVIRUSES

(71) Applicant: The Trustees of Columbia University in the City of New York, New York, NY (US)

(72) Inventors: Masayuki YAZAWA, New York, NY (US); Ramsey BEKDASH, New York, NY (US); Kazushige YOSHIDA, New York, NY (US); David D. HO, New York, NY (US); Yaoxing HUANG, New York, NY (US); Manoj NAIR, New York, NY (US)

(73) Assignee: The Trustees of Columbia University in the City of New York, New York, NY (US)

(21) Appl. No.: 18/912,989

(22) Filed: Oct. 11, 2024

Related U.S. Application Data
(63) Continuation of application No. PCT/US23/18316, filed on Apr. 12, 2023.
(60) Provisional application No. 63/330,408, filed on Apr. 13, 2022.

(51) Int. Cl. C07K 14/005; A61K 38/00; A61P 31/14

(52) U.S. Cl. CPC C07K 14/005; A61P 31/14; A61K 38/00; C07K 2319/10; C12N 2770/20022

(57) ABSTRACT

The present inhibitory polypeptides/peptides are derived from an envelope (E) protein of a coronavirus (e.g., human coronaviruses such as SARS-CoV-2). The polypeptides/peptides against coronaviruses can be used to treat or prevent infection by a coronavirus in a subject.

### CROSS-REFERENCE TO RELATED APPLICATIONS

This present application is a Continuation application of PCT/US23/18316 filed on Apr. 12, 2023, which claims priority to U.S. Provisional Patent Application No. 63/330,408 filed on Apr. 13, 2022, both of which are incorporated herein by reference in their entirety.

### SEQUENCE LISTING

The instant application contains a Sequence Listing which has been submitted electronically in .xml format and is hereby incorporated by reference in its entirety. Said .xml copy, created on Apr. 10, 2023, is named 01001_008889-WOO and is 133,268 bytes in size.

### BACKGROUND

The COVID-19 pandemic caused by the SARS-CoV-2 virus¹⁻³, has affected approximately 750 million people and counting in the world. More than seven million people have passed away due to the viral infection. Because the mutagenesis rate in SARS-CoV-2 genes such as Spike is high⁴⁻⁶, it is a challenge to develop sustainable approaches for prevention and treatment. While several new vaccines and drug candidates have become available, the number of COVID-19 infections and deaths are still increasing, and new variants are being reported⁷⁻⁹. Therefore, there is a great need to target coronavirus genes that are highly conserved in SARS-CoV-1 and SARS-CoV-2 as well as other human coronaviruses.

Human coronaviruses such as SARS-CoV-2 and Middle East Respiratory Syndrome coronavirus (MERS-CoV) express an Envelope (E) protein that forms an ion channel essential for viral function called a viroporin¹⁰⁻¹⁵. Compared to the other molecules, E is highly conserved among coronaviruses: SARS-CoV-2 E (2E) protein has 75 amino-acid residues, high homology with SARS-CoV-1 E protein (˜96%) with identical transmembrane and pore structures^{10,16,17}. 2E induces cellular toxicity in a number of different ways^{11,15,18-20}, and may play essential roles in viral function. 2E can be a potential therapeutic target for COVID-19 and future variants.

### SUMMARY

The present disclosure provides for a fusion polypeptide, comprising (or consisting essentially of, or consisting of): (i) a cell-penetrating peptide; and (ii) a coronavirus peptide comprising a fragment of an envelope (E) protein of a coronavirus.

The present disclosure also provides for a coronavirus peptide comprising a fragment of an envelope (E) protein of a coronavirus.

The coronavirus peptide may have about 10 to about 30 amino acid residues in length, where the coronavirus peptide has an amino acid sequence at least or about 70% identical, at least or about 75% identical, at least or about 80% identical, at least or about 85% identical, at least or about 90% identical, at least or about 95% identical, or about 100% identical, to a consecutive amino acid sequence within position 1 to position 40 of the envelope (E) protein of a coronavirus.

The coronavirus peptide may have about 15 to about 20 amino acid residues in length, where the coronavirus peptide has an amino acid sequence at least or about 70% identical, at least or about 75% identical, at least or about 80% identical, at least or about 85% identical, at least or about 90% identical, at least or about 95% identical, or about 100% identical, to a consecutive amino acid sequence within position 1 to position 25 of the envelope (E) protein of a coronavirus.

The coronavirus peptide may have about 18 amino acid residues in length, and wherein the coronavirus peptide has an amino acid sequence at least or about 70% identical, at least or about 75% identical, at least or about 80% identical, at least or about 85% identical, at least or about 90% identical, at least or about 95% identical, or about 100% identical, to a consecutive amino acid sequence within position 1 to position 18 of the envelope (E) protein of a coronavirus.

In certain embodiments, the coronavirus peptide may comprise at least one (e.g., 1, 2, 3, 4, 5, or more) glutamic acid to aspartic acid mutation, and/or at least one (e.g., 1, 2, 3, 4, 5, or more) aspartic acid to glutamic acid mutation, compared to the wildtype envelope (E) protein of a coronavirus.

In certain embodiments, the coronavirus peptide may comprise (or consist essentially of, or consist of) an amino acid sequence at least or about 70% identical, at least or about 75% identical, at least or about 80% identical, at least or about 85% identical, at least or about 90% identical, at least or about 95% identical, or about 100% identical, to the amino acid sequence set forth in SEQ ID NO: 1, SEQ ID NO: 13, SEQ ID NO: 14, SEQ ID NO: 17, SEQ ID NO: 19, SEQ ID NO: 22, or SEQ ID NO: 24.

In certain embodiments, the coronavirus peptide may comprise (or consist essentially of, or consist of) an amino acid sequence at least or about 70% identical, at least or about 75% identical, at least or about 80% identical, at least or about 85% identical, at least or about 90% identical, at least or about 95% identical, or about 100% identical, to the amino acid sequence set forth in SEQ ID NO: 2, SEQ ID NO: 15, SEQ ID NO: 16, SEQ ID NO: 18, SEQ ID NO: 20, SEQ ID NO: 21, SEQ ID NO: 23 or SEQ ID NO: 25.

In certain embodiments, the cell-penetrating peptide may comprise (or consist essentially of, or consist of) TAT, 6-Arg or Penetratin.

In certain embodiments, the cell-penetrating peptide may comprise (or consist essentially of, or consist of) an amino acid sequence at least or about 70% identical, at least or about 75% identical, at least or about 80% identical, at least or about 85% identical, at least or about 90% identical, at least or about 95% identical, or about 100% identical, to the amino acid sequence set forth in SEQ ID NO: 4, SEQ ID NO: 5 or SEQ ID NO: 6.

In certain embodiments, the cell-penetrating peptide is directly linked to the coronavirus peptide.

In certain embodiments, the fusion polypeptide may further comprise a linker connecting (linking) the cell-penetrating peptide and the coronavirus peptide.

In certain embodiments, the linker is a polyethylene glycol (PEG) linker, such as PEG, (PEG)2, (PEG)3, (PEG)4, (PEG)5, (PEG)6, (PEG)7, (PEG)8, (PEG)9, or (PEG)10. In one embodiment, the linker is (PEG)3.

In certain embodiments, the fusion polypeptide is PEGylated.

In certain embodiments, the coronavirus peptide is PEGylated.

The cell-penetrating peptide may be located at the N-terminus or the C-terminus of the fusion polypeptide. The coronavirus peptide may be located at the C-terminus or the N-terminus of the fusion polypeptide.

The fusion polypeptide may comprise (or consist essentially of, or consist of) an amino acid sequence at least or about 70% identical, at least or about 75% identical, at least or about 80% identical, at least or about 85% identical, at least or about 90% identical, at least or about 95% identical, or about 100% identical, to the amino acid sequence set forth in SEQ ID NO: 7, SEQ ID NO: 9 or SEQ ID NO: 10.

The fusion polypeptide may comprise (or consist essentially of, or consist of) an amino acid sequence at least or about 70% identical, at least or about 75% identical, at least or about 80% identical, at least or about 85% identical, at least or about 90% identical, at least or about 95% identical, or about 100% identical, to the amino acid sequence set forth in SEQ ID NO: 3 or SEQ ID NO: 8.

The present disclosure provides for a nucleic acid encoding the fusion polypeptide or the coronavirus peptide.

Also encompassed by the present disclosure is a pharmaceutical composition comprising the fusion polypeptide or the coronavirus peptide. The present disclosure also provides for a pharmaceutical composition comprising the nucleic acid encoding the fusion polypeptide or the coronavirus peptide.

The present disclosure provides for a kit comprising the fusion polypeptide, the coronavirus peptide, or the pharmaceutical composition.

The present disclosure also provides for a method of treating or preventing infection by a coronavirus in a subject.

The method may comprise administering the fusion polypeptide or the coronavirus peptide to the subject. The method may comprise administering the pharmaceutical composition to the subject. The method may comprise administering the nucleic acid to the subject.

The present polypeptide/peptide, nucleic acid, or pharmaceutical composition may be administered to the subject by nasal administration (intranasal administration), pulmonary administration (e.g., by nebulization), intravenous administration, or oral administration. The present polypeptide/peptide, nucleic acid, or pharmaceutical composition may be administered to the subject by parenteral administration.

The coronavirus may be a human coronavirus, such as SARS-CoV-2, SARS-CoV-1, MERS-CoV, HCoV-229E, HCoV-NL63, HCoV-OC43 or HCoV-HKU1.

In certain embodiments, the envelope (E) protein of a coronavirus comprises (or consists essentially of, or consists of) an amino acid sequence at least or about 70% identical, at least or about 75% identical, at least or about 80% identical, at least or about 85% identical, at least or about 90% identical, at least or about 95% identical, or about 100% identical, to the amino acid sequence set forth in SEQ ID NO: 11, SEQ ID NO: 12, SEQ ID NO: 26, SEQ ID NO: 27, SEQ ID NO: 28, SEQ ID NO: 29, or SEQ ID NO: 30.

### BRIEF DESCRIPTION OF THE DRAWINGS

For the purpose of illustrating the invention, there are depicted in drawings certain embodiments of the invention. However, the invention is not limited to the precise arrangements and instrumentalities of the embodiments depicted in the drawings.

FIGS. 1a-1e: Testing the amino-terminal fragment of SARS-CoV-2 Envelope named MY18 on lysosomal pH imaging in mammalian cells. (FIG. 1a) Alignment of Envelope of SARS-CoV (SARS1-E) and SARS-CoV-2 (SARS2-E), showing the difference in amino acids (dashed line), the targeted amino-terminal region named MY18 and putative transmembrane region. (FIG. 1b) Representative confocal fluorescent and bright field images of NIH 3T3 cells loaded with DND-189, a lysosomal pH fluorescent dye, and transfected with MY18 peptide construct, empty vector (mock) and SARS2-E fused with mKate2 fluorescent protein (2E-mKate2). Scale bar, 5 μm. (FIG. 1c) Relative fluorescent intensity of DND-189 dye in NIH 3T3 cells transfected using mock (n=40) or 2E-mKate2 plasmid without (−, n=36) and with MY18 plasmid (+MY18, n=30). One-way ANOVA with Tukey's multiple comparisons test (**** P<0.0001; n.s. not significant). (FIG. 1d) The sequences of cell-penetrating peptide candidates, 6-Arg (6Arg), TAT and Penetratin (Pen), for MY18 peptide uptake in mammalian cells. (FIG. 1e) Relative fluorescent intensity of DND-189 dye in NIH 3T3 cells incubated with MY18 (10 μM, n=93), 6Arg-MY18 (0.1 μM, n=40; 1, n=41; 10, n=106), TAT-MY18 (0.1 μM, n=27; 1, n=20; 10, n=92) and Pen-MY18 peptides (0.1 μM, n=27; 1, n=32; 10, n=68) with 2E-mKate2 plasmid transfection. Mock (n=116) and non-treated 2E-mKate2 (−, n=139) were also tested as their controls. In 1 μM condition, TAT-MY18 peptide is not different from mock (shaded), while 6Arg and Pen are significantly lower than mock. One-way ANOVA with Dunnett's multiple comparisons test (**** P<0.0001; *** P<0.001; ** P<0.01; n.s. not significant, compared to mock). All the graphs in the figure are mean±s.d.

FIGS. 2a-2g: Mutagenesis of MY18 peptide to develop iPep-SARS2-E. (FIG. 2a) Volcano plots of global proteomics of HEK 293S cells transfected with SARS2-E fused to mKate2 (2E-mKate2). Plots demonstrate significant increases in 2E-mKate2 compared to empty vector (mock) (false discovery rate, FDR: 0.05). (FIG. 2b and FIG. 2c) The expressions of JUN/AP-1 (FIG. 2b) and NFATC4 transcripts (FIG. 2c) significantly increased in HEK 293S cells transfected to 2E-mKate2 compared to mock. Unpaired Student's t-test was used (** P<0.01; * P<0.05, n=3−4). (FIG. 2d) Schematic representation of dual luminescence reporter system using 4-repeated NFAT response element (RE) of human IL-2 gene and Firefly luciferase gene (NFAT-FLuc) and herpes simplex virus thymidine kinase (HSV TK) promoter-driven Renilla luciferase gene (TK-RLuc) as transfection control. (FIG. 2e) Relative NFAT-FLuc/TK-RLuc activity in HEK 293T cells transfected using empty vector (mock, n=9) or 2E-mKate2 plasmid without (−, n=9) and with MY 18 plasmid (+MY18, n=12). One-way ANOVA with Tukey's multiple comparisons test was used (**** P<0.0001). (FIG. 2f) Relative NFAT-FLuc/TK-RLuc activity in HEK 293T cells transfected using empty vector (mock, n=9) or 2E-mKate2 plasmid without (−, n=6) and with various sizes of SARS2-E amino-terminal constructs including MY18 (each, n=6, inset). One-way ANOVA with Dunnett's multiple comparisons test was used (**** P<0.0001; ** P<0.01; * P<0.05; n.s. not significant, compared to MY18). (FIG. 2g) Relative NFAT-FLuc/TK-RLuc activity in HEK 293T cells transfected using empty vector (mock, n=3) or 2E-mKate2 plasmid without (−, n=3) and with MY18 mutants (each, n=3). One-way ANOVA with Dunnett's multiple comparisons test was used (**** P<0.0001; ** P<0.01; * P<0.05; n.s. not significant, compared to MY18 wild-type, WT, n=9). S-T switch, Ser and Thr replaced each other. The inset demonstrates the amino acid sequence of MY18-2ED (EE7-8DD, underlined) mutant, which significantly improves the inhibitory effect on SARS2-E-mediated NFAT-JUN/AP-1 activation. All the graphs in the figure are mean±s.d.

FIGS. 3a-3e: Characterization of iPep-SARS2-E. (FIG. 3a) Representative immunoblot images of HEK 293T cells transfected with SARS2-E fused with YFP (2E-YFP) and 48-hr treated with 10 uM TAT-MY18-2ED or MY18-WT peptides (negative control). Anti-GFP (for 2E-YFP, top) and GAPDH antibodies (as loading control, bottom) were used. Putative aggregates of 2E-YFP proteins (&) were observed even though urea-based lysis buffer was used for the sample preparation. #, non-specific bands around 40 and 50 kDa according to the YFP blotting. (FIG. 3b and FIG. 3c) Quantification of 2E-YFP monomeric form (FIG. 3b) and aggregates (FIG. 3c) of HEK 293T cells transfected with 2E-YFP and treated using TAT-MY18-2ED (n=4) or MY18-WT peptides (n=4). (FIG. 3d) Representative immunoblot images of HEK 293T cells transfected with YFP plasmid and 48-hr treated with TAT-MY18-2ED (10 μM, n=3) and MY18-WT peptides (10 μM, n=3, a negative control) and non-treated cells (n=3, another negative control). Anti-GFP (for YFP, top) and GAPDH antibodies (as loading control, bottom) were used. (FIG. 3e) Representative epi-fluorescence image of NIH 3T3 cells co-transfected with 2E-mKate2 and 6×His-MY18 plasmids. Anti-His tag antibody conjugated to Alexa Fluor 488 and Hoechst 33258 dye (nucleus) were used. White arrowheads, co-localization of mKate2 and Alexa Fluor 488 fluorescence. Scale bar, 5 μm. All the graphs in the figure are mean±s.d.

FIGS. 4a-4d: Permeability of iPep-SARS2-E. (FIG. 4a) Representative fluorescent and bright field images of time-course cell-penetrating test using Alexa Fluor 594(A594)-conjugated iPep-SARS2-E peptides, A594-TAT-MY18-2ED (amino-terminal conjugation, N-term, 1 μM, bottom) and TAT-MY18-2ED-A594 (carboxyl-terminal, C-term, 10 μM, top) in NIH 3T3 cells after the incubation started. Scale bar, 50 μm. (FIG. 4b) Quantification of fluorescence-positive cells treated with the A594-conjugated peptides for the peptide cell-penetrating “on” kinetics (mean±s.d.). (FIG. 4c) Experimental design for the peptide stability, “off” kinetics, quantification. (FIG. 4d) Representative fluorescent and bright field images after washout of A594-conjugated TAT-MY18-2ED peptide (C-term version) in NIH 3T3 cells. White arrowheads, fluorescent puncta. Scale bar, 50 μm.

FIGS. 5a-5n: iPep-SARS2-E in vitro test. (FIG. 5a) Experimental design for the iPep-SARS2-E (TAT-MY18-2ED) test using SARS-CoV-2 WA1 virus (MOI, 0.10) and Vero-E6 cells in vitro. (FIG. 5b) Inhibition of iPep-SARS2-E in the cytopathic effect of SARS-CoV-2 WA1 virus on Vero-E6 cells. MY18 WT (non-TAT) was used as a control. (FIG. 5c) Design of time-course experiment using SARS-CoV-2 WA1 virus (MOI, 0.10) and Vero-E6 cells in vitro. (FIG. 5d) Time-course qPCR of SARS2-CoV-2 nucleocapsid (SARS2 N) expression of PBS- and iPep-SARS2-E (1 μM)-treated Vero-E6 cells. The expression of N gene was normalized to a house-keeping gene, GAPDH. Student's t-test was used at each time point (n.s., not significant). (FIG. 5e) qPCR of SARS2 N expression of PBS- and iPep-SARS2-E (1 μM)-treated Vero-E6 cell culture supernatant (sup) at 24 hr post-infection. Student's t-test was used (**** P<0.0001). (FIG. 5f) qPCR of JUN/AP-1 expression of PBS- and iPep-SARS2-E (1 μM)-treated Vero-E6 cells comparing to non-infected cells. The expression of JUN was normalized to GAPDH. One-way ANOVA with Tukey's multiple comparisons test was used (* P<0.05; n.s., not significant). (FIG. 5g) Representative electron microscopic (EM) image of PBS-treated Vero-E6 cells at 24 hours post-infection. Scale bar, 500 nm. (FIG. 5h) Higher magnification of the EM image of PBS-treated Vero-E6 cells at 24 hours post-infection (a box shown in FIG. 5g). Scale bar, 100 nm. (FIG. 5i) Representative EM image of iPep-SARS2-E-treated Vero-E6 cells at 24 hours post-infection. Arrowheads, virus-like particles accumulated in the endoplasmic reticulum. Scale bar, 500 nm. (FIG. 5j) Representative confocal fluorescent images of Vero-E6 cells treated with PBS or iPep-SARS2-E at 24 hr post-infection. SARS2 N antibody and Hoechst 33258 dye (for nucleus) were used with antibodies of subcellular organelle markers: BiP for endoplasmic reticulum (ER), ERGIC-53 for ER Golgi inter compartment (ERGIC) and LAMP1 for lysosome. Scale bar, 5 μm. (FIG. 5k) Experimental design for the iPep-SARS2-E test using SARS-CoV-2 WA1 virus (MOI, 0.10) and human cerebral organoids in vitro. (FIG. 5l and FIG. 5m) qPCR of SARS2 N expression of PBS- and iPep-SARS2-E (1 μM)-treated organoids culture supernatant (sup, FIG. 5l) and cells (FIG. 5m) at 48 hr post-infection. Student's t-test was used (** P<0.01; * P<0.05). (FIG. 5n) Representative merged section images of epi-fluorescence and bright field of human cerebral organoids treated with PBS or iPep-SARS2-E at 48 hr post-infection. SARS2 N antibody (arrowheads) and DAPI dye (for nucleus) were used. Scale bar, 50 μm. All the graphs in the figure are mean±s.d.

FIGS. 6a-6f: iPep-SARS2-E in vivo preclinical study. (FIG. 6a) Representative fluorescent and bright field images of lung tissues isolated from mice administrated i.v. with PBS or Alexa594-conjugated iPep-SARS2-E peptide (TAT-MY18-2ED-A594, 300 μM, 2 hr and 24 hr). After isolating the tissues, the samples were washed using PBS three times, and the fluorescent and bright field images were taken by a fluorescent stereoscope. Scale bar, 2 mm. (FIG. 6b) Experimental design using the iPep-SARS2-E i.v. injection in vivo. (FIG. 6c) There is no difference in body weight between PBS control (n=11) and iPep-SARS2-E-treated mice (n=7). (FIG. 6d) There is a significant reduction of lung viral titer in iPep-SARS2-E-treated mice compared to the control. Student's t-test was used (* P<0.05; n.s., not significant). Median tissue culture infection dose (TCID) is normalized to lung wet weight (g) measured before tissue homogenization to isolate the virus. (FIG. 6e) iPep-SARS2-E significantly reduced the transcript expression of SARS2 N in MA10-infected Balb/c mouse lung tissues (PBS, n=11; iPep-SARS2-E, n=7, normalized to mouse GAPDH expression). Student's t-test was used (** P<0.01). (FIG. 6f) Representative images of hematoxylin and eosin (H&E) staining of mouse lung tissues of PBS control and iPep-SARS2-E-treated mice at 4 days post-infection. Protein accumulation (x) and immune cells (arrowheads) are indicated. Scale bar, 50 μm. All the graphs in the figure are mean±s.d.

FIGS. 7a-7f: MY18 peptide application for other human coronaviruses. (FIG. 7a) Alignment of the N-terminal region of human coronavirus Envelope (E) of SARS-CoV-2, MERS-CoV, HCoV-229E, HCov-NL63, HCov-OC43 and HCov-HKU1. Glu (E), Asp (D) and other amino acid residues (R, N) are targeted for further mutagenesis to customize MY18 inhibitory peptides for each viral E. (FIG. 7b) MY18 peptide design for each human coronavirus E of SARS-CoV-2, MERS-CoV, HCoV-229E, HCov-NL63, HCov-OC43 and HCov-HKU1. Glu and Asp are replaced (underlined), following iPep-SARS2-E. (FIG. 7c) Relative NFAT-FLuc/TK-RLuc activity in HEK 293T cells transfected using mock (n=17), SARS2-E (n=10) and the other coronavirus E (each, n=8). One-way ANOVA with Dunnett's multiple comparisons test was used (**** P<0.0001; * P<0.05; n.s. not significant, compared to mock). (FIG. 7d) Relative NFAT-FLuc/TK-RLuc activity in HEK 293T cells transfected using mock (n=9), NL63-E without (n=12) and with NL63-MY18 constructs (WT, n=12; 2DE, n=3; the other mutants, n=6). One-way ANOVA with Dunnett's multiple comparisons test was used (**** P<0.0001; *** P<0.001; n.s. not significant, compared to mock). (FIG. 7e) Relative NFAT-FLuc/TK-RLuc activity in HEK 293T cells transfected using mock (n=9), MERS-E without (n=12) and with MERS-MY18 constructs (WT, n=12; E7D, n=3; the other mutants, n=6). One-way ANOVA with Dunnett's multiple comparisons test was used (**** P<0.0001; ** P<0.01; n.s. not significant, compared to mock). (FIG. 7f) Relative fluorescent intensity of DND-189 dye in NIH 3T3 cells transfected with mock (n=78) and the other human coronavirus E-mKate2 constructs: OC43 (n=74), 229E (n=66), MERS (n=63), NL63 (n=65) and HKU1 (n=78). Each MY18 mutant construct was also tested: MERS-MY18 (R8H, n=36), NL63-MY18 (2DE & N9D, n=37) and HKU1-MY18 (D8E, n=31). One-way ANOVA with Dunnett's multiple comparisons test was used (**** P<0.0001; n.s. not significant, compared to mock). All the graphs in the figure are mean±s.d.

FIGS. 8a-8d: iPep-SARS2-E in vitro validation. (FIG. 8a-FIG. 8d) qPCR of SARS-CoV-2 N (FIG. 8a), E (FIG. 8b), JUN/AP-1 expression (FIG. 8c) of PBS (n=6)- and iPep-SARS2-E (1 μM, n=6)-treated Vero-E6 cells comparing to non-infected cells (n=6) at 48 hr post-infection. The expression of these genes was normalized to a house-keeping gene, GAPDH. One-way ANOVA with Tukey's multiple comparisons test was used (**** P<0.0001; *** P<0.001; ** P<0.01; n.s., not significant). (FIG. 8d) qPCR of SARS2-CoV-2 N expression of PBS (n=6)- and iPep-SARS2-E (1 μM, n=6)-treated Vero-E6 cell culture supernatant (sup) at 48 hr post-infection. Student's t-test was used (**** P<0.0001). The cDNA samples of cells and cell culture sup were prepared with TRIzol Plus RNA Purification kit, PureLink DNase set and SuperScript III and then diluted (1/5) using UltraPure distilled water for conducting qPCR.

FIGS. 9a-9e: iPep-SARS2-E in vivo test using intranasal administration. (FIG. 9a) Experimental design for the iPep-SARS2-E test in vivo using intranasal administration. (FIG. 9b) iPep-SARS2-E prevents body weight loss in SARS-CoV-2 MA10-infected Balb/c mice (5.0×10\hat{ }4 PFU/mouse). Student's t-test was used at each day (** P<0.01; * P<0.05). (FIG. 9c-FIG. 9d) Representative immunoblots of SARS-CoV-2 E (2E, FIG. 9c) and mouse Gapdh proteins (FIG. 9d) in SARS-CoV-2 MA10-infected mouse lung tissues with PBS or iPep-SARS2-E treatment. (FIG. 9e) iPep-SARS2-E peptides significantly reduced the protein expression of 2E in MA10-infected Balb/c mouse lung tissues (PBS, n=4; iPep-SARS2-E, n=4). Student's t-test was used (* P<0.05). All the graphs in the figure are mean±s.d.

FIGS. 10a-10d: PEGylated MY18 peptide application for SARS-CoV-2 in vivo. (FIG. 10a) Amino acid sequence of the PEGylated version of iPep-SARS2-E using PEG3 addition between TAT and MY18-2ED (named “iPep-PEG3”). (FIG. 10b) Experimental design of intranasal administration in mouse study using iPep-PEG3. (FIG. 10c) Body weight changes in control mice treated with a mutant peptide (neg. Ctrl, n=5) and mice treated with iPep-PEG3 (n=5). Student's t-test was used at each time-point (*P<0.05). (FIG. 10d) iPep-PEG3 significantly reduced the transcript expression of SARS-CoV-2 N in the infected mouse lung tissues (neg. Ctrl, n=5; iPep-PEG3, n=5). Student's t-test was used (*P<0.05). The graphs in the figures are mean±s.d.

FIG. 11: Envelope sequence alignment of SARS-CoV-2, MERS-CoV, HCoV-229E, HCoV-NL63, HCoV-OC43 and HCoV-HKU1. CLUSTALW 2.1 multiple sequence alignment software is used to obtain the alignment.

### DETAILED DESCRIPTION

The present inhibitory polypeptides/peptides against coronaviruses (e.g., human coronaviruses such as SARS-CoV-2) can be used to treat or prevent infection by a coronavirus in a subject. The subject may be known or suspected of having, or being at risk of, an infection or disease caused by a coronavirus (e.g., a human coronavirus such as SARS-CoV-2) or has been exposed to a coronavirus (e.g., a human coronavirus such as SARS-CoV-2).

The present disclosure provides for a fusion polypeptide, comprising (or consisting essentially of, or consisting of): (i) a cell-penetrating peptide; and (ii) a coronavirus peptide comprising a fragment of an envelope (E) protein of a coronavirus.

In certain embodiments, the cell-penetrating peptide is located at the N-terminus or C-terminus of the fusion polypeptide. In certain embodiments, the coronavirus peptide is located at the C-terminus or N-terminus of the fusion polypeptide.

The cell-penetrating peptide may be directly linked to the coronavirus peptide.

The cell-penetrating peptide may be linked to the coronavirus peptide through a linker. In certain embodiments, the present fusion polypeptide may further comprise a linker linking (or ligating or connecting) the cell-penetrating peptide and the coronavirus peptide.

The fusion polypeptide may comprise (or consist essentially of, or consist of) the following structure (N-terminus to C-terminus):
cell-penetrating peptide—coronavirus peptide, or
coronavirus peptide—cell-penetrating peptide.

The fusion polypeptide may comprise (or consist essentially of, or consist of) the following structure (N-terminus to C-terminus):
cell-penetrating peptide—linker—coronavirus peptide, or
coronavirus peptide—linker—cell-penetrating peptide.

The fusion polypeptide can be designed to place the various functional moieties (a cell-penetrating peptide, and a coronavirus peptide) in any order. In the fusion polypeptide, these functional moieties may be covalently ligated continuously or non-continuously (e.g., they may be separated by linkers (e.g., a peptide linker or linker amino acid residues)). The linker may have up to 30, up to 20, up to 18, up to 15, up to 12, up to 11, or up to 10, amino acid residues in length. In certain embodiments, the linker has about 1, 2, 3, 4, 5, 6, 7, 8, 9, 10-20, 8-10, 8-12, 8-15, 8-20, or 8-30 amino acid residues in length. In certain embodiments, the linker has about 7-10, 7-12, 7-15, 7-20, or 7-30 amino acid residues in length.

The linker may be a peptide linker or a non-peptide linker. In certain embodiments, the linker may be a polyethylene glycol (PEG) linker. In one embodiment, the PEG linker is (PEG)3.

The present disclosure also provides for a coronavirus peptide comprising (or consisting essentially of, or consisting of) a fragment of an envelope (E) protein of a coronavirus.

A fragment as used herein may refer to less than 100% of the sequence (e.g., an envelope (E) protein of a coronavirus), e.g., about 99%, about 95%, about 90%, about 85%, about 80%, about 75%, about 70%, about 65%, about 60%, about 55%, about 50%, about 45%, about 40%, about 35%, about 30%, about 25%, about 20%, about 15%, about 10% etc.), and/or comprising about 5, 6, 7, 8, 9, 10, 12, 13, 14, 15, 16, 17, 18, 19, 20, 21, 22, 23, 24, 25, 26, 27, 28, 29, 30, 31, 32, 33, 34, 35, 36, 37, 38, 39, 40, or more amino acid residues of the sequence (e.g., an envelope (E) protein of a coronavirus).

The present polypeptide/peptide may be PEGylated.

In certain embodiments, the fusion polypeptide may comprise (or consist essentially of, or consist of) a cell-penetrating peptide and a coronavirus peptide, where the coronavirus peptide may comprise (or consist essentially of, or consist of) an amino acid sequence at least or about 50%, at least or about 55%, at least or about 60%, at least or about 61%, at least or about 62%, at least or about 63%, at least or about 64%, at least or about 65%, at least or about 66%, at least or about 67%, at least or about 68%, at least or about 69%, at least or about 70%, at least or about 71%, at least or about 72%, at least or about 73%, at least or about 74%, at least or about 75%, at least or about 76%, at least or about 77%, at least or about 78%, at least or about 79%, at least or about 80%, at least or about 81%, at least or about 82%, at least or about 83%, at least or about 84%, at least or about 85%, at least or about 86%, at least or about 87%, at least or about 88%, at least or about 89%, at least or about 90%, at least or about 91%, at least or about 92%, at least or about 93%, at least or about 94%, at least or about 95%, at least or about 96%, at least or about 97%, at least or about 98%, at least or about 99%, or about 100%, identical to the amino acid sequence set forth in SEQ ID NO: 1, SEQ ID NO: 13, SEQ ID NO: 14, SEQ ID NO: 17, SEQ ID NO: 19, SEQ ID NO: 22, SEQ ID NO: 24, SEQ ID NO: 2, SEQ ID NO: 15, SEQ ID NO: 16, SEQ ID NO: 18, SEQ ID NO: 20, SEQ ID NO: 21, SEQ ID NO: 23 or SEQ ID NO: 25, or fragments, variants, analogs, orthologs, homologs or derivatives thereof; and where the cell-penetrating peptide may comprise (or consist essentially of, or consist of) an amino acid sequence at least or about 50%, at least or about 55%, at least or about 60%, at least or about 61%, at least or about 62%, at least or about 63%, at least or about 64%, at least or about 65%, at least or about 66%, at least or about 67%, at least or about 68%, at least or about 69%, at least or about 70%, at least or about 71%, at least or about 72%, at least or about 73%, at least or about 74%, at least or about 75%, at least or about 76%, at least or about 77%, at least or about 78%, at least or about 79%, at least or about 80%, at least or about 81%, at least or about 82%, at least or about 83%, at least or about 84%, at least or about 85%, at least or about 86%, at least or about 87%, at least or about 88%, at least or about 89%, at least or about 90%, at least or about 91%, at least or about 92%, at least or about 93%, at least or about 94%, at least or about 95%, at least or about 96%, at least or about 97%, at least or about 98%, at least or about 99%, or about 100%, identical to the amino acid sequence set forth in SEQ ID NO: 4, SEQ ID NO: 5, SEQ ID NO: 6, or any of SEQ ID NOs: 45-148, or fragments, variants, analogs, orthologs, homologs or derivatives thereof.

In certain embodiments, the fusion polypeptide may comprise (or consist essentially of, or consist of) a cell-penetrating peptide and a coronavirus peptide, where the coronavirus peptide may comprise (or consist essentially of, or consist of) an amino acid sequence at least or about 50%, at least or about 55%, at least or about 60%, at least or about 61%, at least or about 62%, at least or about 63%, at least or about 64%, at least or about 65%, at least or about 66%, at least or about 67%, at least or about 68%, at least or about 69%, at least or about 70%, at least or about 71%, at least or about 72%, at least or about 73%, at least or about 74%, at least or about 75%, at least or about 76%, at least or about 77%, at least or about 78%, at least or about 79%, at least or about 80%, at least or about 81%, at least or about 82%, at least or about 83%, at least or about 84%, at least or about 85%, at least or about 86%, at least or about 87%, at least or about 88%, at least or about 89%, at least or about 90%, at least or about 91%, at least or about 92%, at least or about 93%, at least or about 94%, at least or about 95%, at least or about 96%, at least or about 97%, at least or about 98%, at least or about 99%, or about 100%, identical to the amino acid sequence set forth in SEQ ID NO: 2, or SEQ ID NO: 1, or fragments, variants, analogs, orthologs, homologs or derivatives thereof; and where the cell-penetrating peptide may comprise (or consist essentially of, or consist of) an amino acid sequence at least or about 50%, at least or about 55%, at least or about 60%, at least or about 61%, at least or about 62%, at least or about 63%, at least or about 64%, at least or about 65%, at least or about 66%, at least or about 67%, at least or about 68%, at least or about 69%, at least or about 70%, at least or about 71%, at least or about 72%, at least or about 73%, at least or about 74%, at least or about 75%, at least or about 76%, at least or about 77%, at least or about 78%, at least or about 79%, at least or about 80%, at least or about 81%, at least or about 82%, at least or about 83%, at least or about 84%, at least or about 85%, at least or about 86%, at least or about 87%, at least or about 88%, at least or about 89%, at least or about 90%, at least or about 91%, at least or about 92%, at least or about 93%, at least or about 94%, at least or about 95%, at least or about 96%, at least or about 97%, at least or about 98%, at least or about 99%, or about 100%, identical to the amino acid sequence set forth in SEQ ID NO: 4 or SEQ ID NO: 55, or fragments, variants, analogs, orthologs, homologs or derivatives thereof.

In certain embodiments, the fusion polypeptide may comprise (or consist essentially of, or consist of) a cell-penetrating peptide and a coronavirus peptide, where the coronavirus peptide may comprise (or consist essentially of, or consist of) an amino acid sequence at least or about 50%, at least or about 55%, at least or about 60%, at least or about 61%, at least or about 62%, at least or about 63%, at least or about 64%, at least or about 65%, at least or about 66%, at least or about 67%, at least or about 68%, at least or about 69%, at least or about 70%, at least or about 71%, at least or about 72%, at least or about 73%, at least or about 74%, at least or about 75%, at least or about 76%, at least or about 77%, at least or about 78%, at least or about 79%, at least or about 80%, at least or about 81%, at least or about 82%, at least or about 83%, at least or about 84%, at least or about 85%, at least or about 86%, at least or about 87%, at least or about 88%, at least or about 89%, at least or about 90%, at least or about 91%, at least or about 92%, at least or about 93%, at least or about 94%, at least or about 95%, at least or about 96%, at least or about 97%, at least or about 98%, at least or about 99%, or about 100%, identical to the amino acid sequence set forth in SEQ ID NO: 1, SEQ ID NO: 13, SEQ ID NO: 14, SEQ ID NO: 17, SEQ ID NO: 19, SEQ ID NO: 22, SEQ ID NO: 24, SEQ ID NO: 2, SEQ ID NO: 15, SEQ ID NO: 16, SEQ ID NO: 18, SEQ ID NO: 20, SEQ ID NO: 21, SEQ ID NO: 23 or SEQ ID NO: 25, or fragments, variants, analogs, orthologs, homologs and derivatives thereof; and where the cell-penetrating peptide may be TAT, HIV-1 Tat, 6-Arg or Penetratin, or fragments, variants, analogs, orthologs, homologs or derivatives thereof.

In certain embodiments, the fusion polypeptide may comprise (or consist essentially of, or consist of) a cell-penetrating peptide and a coronavirus peptide, where the coronavirus peptide may comprise (or consist essentially of, or consist of) an amino acid sequence at least or about 50%, at least or about 55%, at least or about 60%, at least or about 61%, at least or about 62%, at least or about 63%, at least or about 64%, at least or about 65%, at least or about 66%, at least or about 67%, at least or about 68%, at least or about 69%, at least or about 70%, at least or about 71%, at least or about 72%, at least or about 73%, at least or about 74%, at least or about 75%, at least or about 76%, at least or about 77%, at least or about 78%, at least or about 79%, at least or about 80%, at least or about 81%, at least or about 82%, at least or about 83%, at least or about 84%, at least or about 85%, at least or about 86%, at least or about 87%, at least or about 88%, at least or about 89%, at least or about 90%, at least or about 91%, at least or about 92%, at least or about 93%, at least or about 94%, at least or about 95%, at least or about 96%, at least or about 97%, at least or about 98%, at least or about 99%, or about 100%, identical to the amino acid sequence set forth in SEQ ID NO: 2, or SEQ ID NO: 1, or fragments, variants, analogs, orthologs, homologs or derivatives thereof; and where the cell-penetrating peptide may be TAT, or HIV-1 Tat, or fragments, variants, analogs, orthologs, homologs or derivatives thereof.

In certain embodiments, the fusion polypeptide may comprise (or consist essentially of, or consist of) a cell-penetrating peptide, a linker, and a coronavirus peptide, where the coronavirus peptide may comprise (or consist essentially of, or consist of) an amino acid sequence at least or about 50%, at least or about 55%, at least or about 60%, at least or about 61%, at least or about 62%, at least or about 63%, at least or about 64%, at least or about 65%, at least or about 66%, at least or about 67%, at least or about 68%, at least or about 69%, at least or about 70%, at least or about 71%, at least or about 72%, at least or about 73%, at least or about 74%, at least or about 75%, at least or about 76%, at least or about 77%, at least or about 78%, at least or about 79%, at least or about 80%, at least or about 81%, at least or about 82%, at least or about 83%, at least or about 84%, at least or about 85%, at least or about 86%, at least or about 87%, at least or about 88%, at least or about 89%, at least or about 90%, at least or about 91%, at least or about 92%, at least or about 93%, at least or about 94%, at least or about 95%, at least or about 96%, at least or about 97%, at least or about 98%, at least or about 99%, or about 100%, identical to the amino acid sequence set forth in SEQ ID NO: 1, SEQ ID NO: 13, SEQ ID NO: 14, SEQ ID NO: 17, SEQ ID NO: 19, SEQ ID NO: 22, SEQ ID NO: 24, SEQ ID NO: 2, SEQ ID NO: 15, SEQ ID NO: 16, SEQ ID NO: 18, SEQ ID NO: 20, SEQ ID NO: 21, SEQ ID NO: 23 or SEQ ID NO: 25, or fragments, variants, analogs, orthologs, homologs or derivatives thereof; where the cell-penetrating peptide may comprise (or consist essentially of, or consist of) an amino acid sequence at least or about 50%, at least or about 55%, at least or about 60%, at least or about 61%, at least or about 62%, at least or about 63%, at least or about 64%, at least or about 65%, at least or about 66%, at least or about 67%, at least or about 68%, at least or about 69%, at least or about 70%, at least or about 71%, at least or about 72%, at least or about 73%, at least or about 74%, at least or about 75%, at least or about 76%, at least or about 77%, at least or about 78%, at least or about 79%, at least or about 80%, at least or about 81%, at least or about 82%, at least or about 83%, at least or about 84%, at least or about 85%, at least or about 86%, at least or about 87%, at least or about 88%, at least or about 89%, at least or about 90%, at least or about 91%, at least or about 92%, at least or about 93%, at least or about 94%, at least or about 95%, at least or about 96%, at least or about 97%, at least or about 98%, at least or about 99%, or about 100%, identical to the amino acid sequence set forth in SEQ ID NO: 4, SEQ ID NO: 5, SEQ ID NO: 6, or any of SEQ ID NOs: 45-148, or fragments, variants, analogs, orthologs, homologs or derivatives thereof; and where the linker may comprise (or consist essentially of, or consist of) PEG, (PEG)2, (PEG)3, (PEG)4, (PEG)5, (PEG)6, (PEG)7, (PEG)8, (PEG)9, (PEG)10, (PEG)11, (PEG)12, (PEG)13, (PEG)14, (PEG)15, PEG containing more than 15 monomer units, a 7-atom polyethylene glycol linker, 8-atom polyethylene glycol linker, 9-atom polyethylene glycol linker, 13-atom polyethylene glycol linker, 14-atom polyethylene glycol linker, 16-atom polyethylene glycol linker, 18-atom polyethylene glycol linker, 20-atom polyethylene glycol linker, 24-atom polyethylene glycol linker, 30-atom polyethylene glycol linker, or 31-atom polyethylene glycol linker.

In certain embodiments, the fusion polypeptide may comprise (or consist essentially of, or consist of) a cell-penetrating peptide, a linker, and a coronavirus peptide, where the coronavirus peptide may comprise (or consist essentially of, or consist of) an amino acid sequence at least or about 50%, at least or about 55%, at least or about 60%, at least or about 61%, at least or about 62%, at least or about 63%, at least or about 64%, at least or about 65%, at least or about 66%, at least or about 67%, at least or about 68%, at least or about 69%, at least or about 70%, at least or about 71%, at least or about 72%, at least or about 73%, at least or about 74%, at least or about 75%, at least or about 76%, at least or about 77%, at least or about 78%, at least or about 79%, at least or about 80%, at least or about 81%, at least or about 82%, at least or about 83%, at least or about 84%, at least or about 85%, at least or about 86%, at least or about 87%, at least or about 88%, at least or about 89%, at least or about 90%, at least or about 91%, at least or about 92%, at least or about 93%, at least or about 94%, at least or about 95%, at least or about 96%, at least or about 97%, at least or about 98%, at least or about 99%, or about 100%, identical to the amino acid sequence set forth in SEQ ID NO: 2, or SEQ ID NO: 1, or fragments, variants, analogs, orthologs, homologs or derivatives thereof; where the cell-penetrating peptide may comprise (or consist essentially of, or consist of) an amino acid sequence at least or about 50%, at least or about 55%, at least or about 60%, at least or about 61%, at least or about 62%, at least or about 63%, at least or about 64%, at least or about 65%, at least or about 66%, at least or about 67%, at least or about 68%, at least or about 69%, at least or about 70%, at least or about 71%, at least or about 72%, at least or about 73%, at least or about 74%, at least or about 75%, at least or about 76%, at least or about 77%, at least or about 78%, at least or about 79%, at least or about 80%, at least or about 81%, at least or about 82%, at least or about 83%, at least or about 84%, at least or about 85%, at least or about 86%, at least or about 87%, at least or about 88%, at least or about 89%, at least or about 90%, at least or about 91%, at least or about 92%, at least or about 93%, at least or about 94%, at least or about 95%, at least or about 96%, at least or about 97%, at least or about 98%, at least or about 99%, or about 100%, identical to the amino acid sequence set forth in SEQ ID NO: 4, or SEQ ID NO: 55, or fragments, variants, analogs, orthologs, homologs or derivatives thereof; and where the linker may comprise (or consist essentially of, or consist of) PEG, (PEG)2, (PEG)3, (PEG)4, (PEG)5, (PEG)6, (PEG)7, (PEG)8, (PEG)9, (PEG)10, (PEG)11, (PEG)12, (PEG)13, (PEG)14, (PEG)15, PEG containing more than 15 monomer units, a 7-atom polyethylene glycol linker, 8-atom polyethylene glycol linker, 9-atom polyethylene glycol linker, 13-atom polyethylene glycol linker, 14-atom polyethylene glycol linker, 16-atom polyethylene glycol linker, 18-atom polyethylene glycol linker, 20-atom polyethylene glycol linker, 24-atom polyethylene glycol linker, 30-atom polyethylene glycol linker, or 31-atom polyethylene glycol linker.

In certain embodiments, the fusion polypeptide may comprise (or consist essentially of, or consist of) a cell-penetrating peptide, a linker, and a coronavirus peptide, where the coronavirus peptide may comprise (or consist essentially of, or consist of) an amino acid sequence at least or about 50%, at least or about 55%, at least or about 60%, at least or about 61%, at least or about 62%, at least or about 63%, at least or about 64%, at least or about 65%, at least or about 66%, at least or about 67%, at least or about 68%, at least or about 69%, at least or about 70%, at least or about 71%, at least or about 72%, at least or about 73%, at least or about 74%, at least or about 75%, at least or about 76%, at least or about 77%, at least or about 78%, at least or about 79%, at least or about 80%, at least or about 81%, at least or about 82%, at least or about 83%, at least or about 84%, at least or about 85%, at least or about 86%, at least or about 87%, at least or about 88%, at least or about 89%, at least or about 90%, at least or about 91%, at least or about 92%, at least or about 93%, at least or about 94%, at least or about 95%, at least or about 96%, at least or about 97%, at least or about 98%, at least or about 99%, or about 100%, identical to the amino acid sequence set forth in SEQ ID NO: 1, SEQ ID NO: 13, SEQ ID NO: 14, SEQ ID NO: 17, SEQ ID NO: 19, SEQ ID NO: 22, SEQ ID NO: 24, SEQ ID NO: 2, SEQ ID NO: 15, SEQ ID NO: 16, SEQ ID NO: 18, SEQ ID NO: 20, SEQ ID NO: 21, SEQ ID NO: 23 or SEQ ID NO: 25, or fragments, variants, analogs, orthologs, homologs or derivatives thereof; where the cell-penetrating peptide may be TAT, HIV-1 Tat, 6-Arg or Penetratin, or fragments, variants, analogs, orthologs, homologs or derivatives thereof; and where the linker may comprise (or consist essentially of, or consist of) PEG, (PEG)2, (PEG)3, (PEG)4, (PEG)5, (PEG)6, (PEG)7, (PEG)8, (PEG)9, (PEG)10, (PEG)11, (PEG)12, (PEG)13, (PEG)14, (PEG)15, PEG containing more than 15 monomer units, a 7-atom polyethylene glycol linker, 8-atom polyethylene glycol linker, 9-atom polyethylene glycol linker, 13-atom polyethylene glycol linker, 14-atom polyethylene glycol linker, 16-atom polyethylene glycol linker, 18-atom polyethylene glycol linker, 20-atom polyethylene glycol linker, 24-atom polyethylene glycol linker, 30-atom polyethylene glycol linker, or 31-atom polyethylene glycol linker.

In certain embodiments, the fusion polypeptide may comprise (or consist essentially of, or consist of) a cell-penetrating peptide, a linker, and a coronavirus peptide, where the coronavirus peptide may comprise (or consist essentially of, or consist of) an amino acid sequence at least or about 50%, at least or about 55%, at least or about 60%, at least or about 61%, at least or about 62%, at least or about 63%, at least or about 64%, at least or about 65%, at least or about 66%, at least or about 67%, at least or about 68%, at least or about 69%, at least or about 70%, at least or about 71%, at least or about 72%, at least or about 73%, at least or about 74%, at least or about 75%, at least or about 76%, at least or about 77%, at least or about 78%, at least or about 79%, at least or about 80%, at least or about 81%, at least or about 82%, at least or about 83%, at least or about 84%, at least or about 85%, at least or about 86%, at least or about 87%, at least or about 88%, at least or about 89%, at least or about 90%, at least or about 91%, at least or about 92%, at least or about 93%, at least or about 94%, at least or about 95%, at least or about 96%, at least or about 97%, at least or about 98%, at least or about 99%, or about 100%, identical to the amino acid sequence set forth in SEQ ID NO: 2, or SEQ ID NO: 1, or fragments, variants, analogs, orthologs, homologs or derivatives thereof; where the cell-penetrating peptide may be TAT, or HIV-1 Tat, or fragments, variants, analogs, orthologs, homologs or derivatives thereof; and where the linker may comprise (or consist essentially of, or consist of) PEG, (PEG)2, (PEG)3, (PEG)4, (PEG)5, (PEG)6, (PEG)7, (PEG)8, (PEG)9, (PEG)10, (PEG)11, (PEG)12, (PEG)13, (PEG)14, (PEG)15, PEG containing more than 15 monomer units, a 7-atom polyethylene glycol linker, 8-atom polyethylene glycol linker, 9-atom polyethylene glycol linker, 13-atom polyethylene glycol linker, 14-atom polyethylene glycol linker, 16-atom polyethylene glycol linker, 18-atom polyethylene glycol linker, 20-atom polyethylene glycol linker, 24-atom polyethylene glycol linker, 30-atom polyethylene glycol linker, or 31-atom polyethylene glycol linker.

In certain embodiments, the present fusion polypeptide/peptide may comprise (or consist essentially of, or consist of) an amino acid sequence at least or about 50%, at least or about 55%, at least or about 60%, at least or about 61%, at least or about 62%, at least or about 63%, at least or about 64%, at least or about 65%, at least or about 66%, at least or about 67%, at least or about 68%, at least or about 69%, at least or about 70%, at least or about 71%, at least or about 72%, at least or about 73%, at least or about 74%, at least or about 75%, at least or about 76%, at least or about 77%, at least or about 78%, at least or about 79%, at least or about 80%, at least or about 81%, at least or about 82%, at least or about 83%, at least or about 84%, at least or about 85%, at least or about 86%, at least or about 87%, at least or about 88%, at least or about 89%, at least or about 90%, at least or about 91%, at least or about 92%, at least or about 93%, at least or about 94%, at least or about 95%, at least or about 96%, at least or about 97%, at least or about 98%, at least or about 99%, or about 100%, identical to the amino acid sequence set forth in SEQ ID NO: 7, SEQ ID NO: 9, or SEQ ID NO: 10, or fragments, variants, analogs, orthologs, homologs or derivatives thereof.

In certain embodiments, the present fusion polypeptide/peptide may comprise (or consist essentially of, or consist of) an amino acid sequence at least or about 50%, at least or about 55%, at least or about 60%, at least or about 61%, at least or about 62%, at least or about 63%, at least or about 64%, at least or about 65%, at least or about 66%, at least or about 67%, at least or about 68%, at least or about 69%, at least or about 70%, at least or about 71%, at least or about 72%, at least or about 73%, at least or about 74%, at least or about 75%, at least or about 76%, at least or about 77%, at least or about 78%, at least or about 79%, at least or about 80%, at least or about 81%, at least or about 82%, at least or about 83%, at least or about 84%, at least or about 85%, at least or about 86%, at least or about 87%, at least or about 88%, at least or about 89%, at least or about 90%, at least or about 91%, at least or about 92%, at least or about 93%, at least or about 94%, at least or about 95%, at least or about 96%, at least or about 97%, at least or about 98%, at least or about 99%, or about 100%, identical to the amino acid sequence set forth in SEQ ID NO: 3 or SEQ ID NO: 8, or fragments, variants, analogs, orthologs, homologs or derivatives thereof.

The present polypeptides or peptides may include fragments, variants, analogs, orthologs, homologs and derivatives of amino acids and/or peptides. The present polypeptides or peptides may contain one or more analogs of amino acids (including, for example, non-naturally occurring amino acids, amino acids which only occur naturally in an unrelated biological system, modified amino acids etc.), peptides with substituted linkages, as well as other modifications known in the art. The present polypeptides or peptides may comprise a peptidomimetic, such as a peptoid. The present polypeptides or peptides may contain one or more amino acid residues modified by, e.g., PEGylation, glycosylation, acylation (e.g., acetylation, formylation, myristoylation, palmitoylation, lipoylation), alkylation (e.g., methylation), isoprenylation or prenylation (e.g., farnesylation, geranylgeranylation), sulfation, amidation, hydroxylation, succinylation, etc. A polypeptide or peptide may be modified by acetylation and/or methylation. The present polypeptide or peptide may be glycosylated, sulfonated and/or phosphorylated and/or grafted to complex sugars or to a lipophilic compound such as, for example, a polycarbon chain or a cholesterol derivative.

In some embodiments, the polypeptide or peptide is PEGylated. In some embodiments, the polypeptide or peptide is glycosylated. In other embodiments, the polypeptide or peptide is non-glycosylated.

The present polypeptide or peptide may further include a moiety (e.g., tag) useful for polypeptide production and/or detection, including, but not limited to, poly-histidine (e.g., six histidine residues) a maltose binding protein, GST, green fluorescent protein (GFP), hemagglutinin, or alkaline phosphatase, secretion signal peptides (e.g., preprotyrypsin signal sequence), c-Myc, and/or FLAG.

The present polypeptide or peptide can be derivatized or linked to another functional molecule. For example, present fusion polypeptide can be functionally linked (by chemical coupling, genetic fusion, noncovalent interaction, etc.) to one or more other molecular entities, such as an antibody or antibody fragment, a detectable agent, an immunosuppressant, a cytotoxic agent, a pharmaceutical agent, a protein or peptide that can mediate association with another molecule (such as a streptavidin core region or a poly-histidine tag), amino acid linkers, signal sequences, immunogenic carriers, or ligands useful in protein purification, such as glutathione-S-transferase, histidine tag, and staphylococcal protein A. Cytotoxic agents may include radioactive isotopes, chemotherapeutic agents, and toxins such as enzymatically active toxins of bacterial, fungal, plant, or animal origin, and fragments thereof. Useful detectable agents with which a protein can be derivatized (or labeled) include fluorescent agents, various enzymes, prosthetic groups, luminescent materials, bioluminescent materials, and radioactive materials. Non-limiting, exemplary fluorescent detectable agents include fluorescein, fluorescein isothiocyanate, rhodamine, and phycoerythrin. A polypeptide or peptide can also be derivatized with detectable enzymes, such as alkaline phosphatase, horseradish peroxidase, beta-galactosidase, acetylcholinesterase, glucose oxidase and the like. A polypeptide can also be derivatized with a prosthetic group (e.g., streptavidin/biotin and avidin/biotin).

One type of derivatized protein is produced by crosslinking/linking two or more polypeptides or peptides (of the same type or of different types). Suitable crosslinkers include those that are heterobifunctional, having two distinct reactive groups separated by an appropriate spacer (e.g., m-maleimidobenzoyl-N-hydroxysuccinimide ester) or homobifunctional (e.g., disuccinimidyl suberate).

A variant of a polypeptide or peptide may be a polypeptide or peptide modified by, for example, the deletion, addition and/or substitution of 1 or more amino acid residues (10 or fewer, 9 or fewer, 8 or fewer, 7 or fewer, 6 or fewer, 5 or fewer, 4 or fewer, 3 or fewer, 2 or fewer, amino acid residues or 1 amino acid residue) of a wildtype protein, polypeptide or peptide.

The number of amino acid insertions, deletions and/or substitutions may be at least or about 1, at least or about 2, at least or about 3, at least or about 4, at least or about 5, at least or about 6, at least or about 7, at least or about 8, at least or about 9, at least or about 10, at least or about 11, at least or about 12, at least or about 13, at least or about 14, at least or about 15, at least or about 16, at least or about 17, at least or about 18, at least or about 19, at least or about 20, at least or about 21, at least or about 22, at least or about 23, at least or about 24, at least or about 25, at least or about 26, at least or about 27, at least or about 28, at least or about 29, at least or about 30 amino acids.

Suitable amino acid substitutions include, but are not limited to, amino acid substitutions known in the art as “conservative”. A “conservative” substitution is one in which an amino acid is substituted for another amino acid that has similar properties, such that one skilled in the art of peptide chemistry would expect the biological activity, secondary structure and/or hydropathic nature of the polypeptide to be substantially unchanged. Amino acid substitutions may be made on the basis of similarity in polarity, charge, solubility, hydrophobicity, hydrophilicity and/or the amphipathic nature of the residues. For example, negatively charged amino acids include aspartic acid and glutamic acid; positively charged amino acids include lysine, histidine and arginine; and amino acids with uncharged polar head groups having similar hydrophilicity values include leucine, isoleucine and valine; glycine and alanine; asparagine and glutamine; and serine, threonine, phenylalanine and tyrosine. A polypeptide/peptide variant may also, or alternatively, contain non-conservative amino acid changes.

Amino acid substitutions may include the substitution of the amino acid residues within one of the groups including: (1) Ala, Pro, Gly (glycine or G), Glu (glutamic acid or E), Asp (aspartic acid or D), Gln (glutamine or Q), Asn (asparagine or N), Ser, Thr; (2) Cys, Ser, Tyr, Thr; (3) Val, Ile, Leu, Met, Ala, Phe; (4) Lys, Arg (arginine or R), His (histidine or H); and (5) Phe, Tyr, Trp, His.

As used herein, the term variant also denotes any peptide, polypeptide, pseudopeptide (peptide incorporating non-biochemical elements) or protein differing from the wildtype protein, polypeptide, or peptide, obtained by one or more genetic and/or chemical modifications. Genetic and/or chemical modification may be understood to mean any mutation, substitution, deletion, addition and/or modification of one or more residues of the protein, polypeptide, or peptide considered. Chemical modification may refer to any modification of the peptide, polypeptide, or protein generated by chemical reaction or by chemical grafting of biological or non-biological molecule(s) onto any number of residues of the protein.

A homolog of a polypeptide or peptide may refer to a polypeptide or peptide from a different species but sharing substantially the same biological function or activity as the corresponding polypeptide or peptide from another species. A homologous sequence may be a polypeptide modified by the addition, deletion or substitution of amino acids, said modification not substantially altering the functional characteristics compared with the unmodified polypeptide. Where homologous sequence indicates sequence identity, it may mean a sequence which presents a high sequence identity (at least or about 70%, at least or about 75%, at least or about 80%, at least or about 85%, at least or about 90%, at least or about 95%, or at least or about 98% sequence identity) with the parent sequence.

As used herein, a homologous sequence or a homologue may comprise additions, deletions or substitutions of one or more amino acids, which do not substantially alter the functional characteristics of the polypeptides/peptides. The number of amino acid deletions or substitutions may be up to 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69 or 70 amino acids.

Also encompassed by the present disclosure is a nucleic acid encoding the present polypeptide or peptide. The nucleic acid may comprise a sequence encoding the present polypeptide or peptide. The nucleic acid may be DNA or RNA. The nucleic acid may be in the form of, be present in and/or be part of a genetic construct. Such genetic constructs generally comprise at least one nucleic acid that is optionally linked to one or more elements of genetic constructs known, such as for example one or more suitable regulatory elements (such as a suitable promoter(s), enhancer(s), terminator(s), etc.) and the further elements of genetic constructs referred to herein. The nucleic acid may be in the form of, be present in and/or be part of a vector, such as for example a plasmid, cosmid, YAC, a viral vector or transposon, which again may be in essentially isolated form. The vector may be an expression vector, i.e., a vector that can provide for expression in vitro and/or in vivo. In addition, the present disclosure provides vectors comprising such nucleic acids, and host cells comprising such vectors. In certain embodiments, the vector is a shuttle vector. In other embodiments, the vector is an expression vector (e.g., a bacterial or eukaryotic expression vector). In certain embodiments, the host cell is a bacterial cell. In other embodiments, the host cell is a eukaryotic cell. The nucleic acid may be in a form suitable for transformation of the intended host cell or host organism, in a form suitable for integration into the genomic DNA of the intended host cell or in a form suitable for independent replication, maintenance and/or inheritance in the intended host organism, or in a form suitable for genetic immunization. The nucleic acids and/or the genetic constructs disclosed herein may be used to transform a host cell or host organism, i.e., for expression and/or production of the polypeptide or peptide. Suitable hosts or host cells will be clear to the skilled person, and may for example be any suitable fungal, prokaryotic or eukaryotic cell or cell line or any suitable fungal, prokaryotic or eukaryotic organism.

In another aspect, the disclosure relates to host or host cell that expresses or that is capable of expressing the present polypeptide or peptide; and/or that contains a nucleic acid encoding such. Non-limiting examples of such hosts or host cells include, but are not limited to, bacterial cells such as, for example, E. coli, yeast cells such as, for example, S. cerevisiae, P. pastoris, insect cells or mammal cells, such as COS7-cells, a CHO cell, a NIH-3T3 cell, or a HEK-293 cell.

The present disclosure provides for a pharmaceutical composition comprising the present polypeptide, peptide, or a nucleic acid encoding the polypeptide/peptide.

The present disclosure provides for a kit comprising the present polypeptide, peptide, a nucleic acid encoding the polypeptide/peptide, or the pharmaceutical composition.

The present disclosure provides for a method of treating, preventing and/or alleviating the symptoms of an infection or disease caused by a coronavirus (e.g., a human coronavirus such as SARS-CoV-2) in a subject. The method may comprise administering the fusion polypeptide, the coronavirus peptide, or the nucleic acid, to the subject. The method may comprise administering the pharmaceutical composition to the subject.

The coronavirus may be a human coronavirus. The coronavirus may be SARS-CoV-2, SARS-CoV-1, MERS-CoV, HCoV-229E, HCoV-NL63, HCoV-OC43 or HCoV-HKU1.

The present methods may further comprise administering a protease inhibitor and/or a polymerase inhibitor to the subject.

Coronaviruses are enveloped, single-stranded RNA viruses which can be pathogenic in animal and human populations. In humans, coronaviruses induce pathogenic respiratory diseases, notably SARS, MERS and more recently COVID-19. Bovine coronavirus is a major cause of calf scours, winter dysentery in adult cows and cause a significant percentage of bovine respiratory disease. Coronaviruses constitute the subfamily Orthocoronavirinae, in the family Coronaviridae, order Nidovirales and realm Riboviria.

The coronavirus may be Alphacoronavirus (Alpha-CoV or α-CoV), Betacoronavirus (Beta-CoV or β-CoV), Deltacoronavirus (Delta-CoV or δ-CoV), or Gammacoronavirus (Gamma-CoV or γ-CoV).

The coronavirus may be human coronavirus. In certain embodiments, the coronavirus may be severe acute respiratory syndrome coronavirus 2 (SARS-CoV-2), severe acute respiratory syndrome coronavirus 1 (SARS-CoV-1), Middle East respiratory syndrome-related coronavirus (MERS-CoV), human coronavirus OC43 (HCoV-OC43), human coronavirus HKU1 (HCoV-HKU1), human coronavirus 229E (HCoV-229E), or human coronavirus NL63 (HCoV-NL63).

The coronavirus envelope (E) proteins are embedded in the lipid bilayer. The E protein may contain a short hydrophilic N-terminal region, a hydrophobic helical transmembrane domain, and a C-terminal region. The E proteins may form pentameric (five-molecular) ion channels in the lipid bilayer. They may be involved in virion assembly, intracellular trafficking, morphogenesis (budding) and egress.

In certain embodiments, the coronavirus envelope (E) protein comprises (or consists essentially of, or consists of) an amino acid sequence at least or about 80%, at least or about 85%, at least or about 90%, or at least or about 95%, identical to the amino acid sequence set forth in SEQ ID NO: 11, SEQ ID NO: 12, SEQ ID NO: 26, SEQ ID NO: 27, SEQ ID NO: 28, SEQ ID NO: 29, or SEQ ID NO: 30. In certain embodiments, the coronavirus envelope (E) protein comprises (or consists essentially of, or consists of) an amino acid sequence at least or about 50%, at least or about 55%, at least or about 60%, at least or about 61%, at least or about 62%, at least or about 63%, at least or about 64%, at least or about 65%, at least or about 66%, at least or about 67%, at least or about 68%, at least or about 69%, at least or about 70%, at least or about 71%, at least or about 72%, at least or about 73%, at least or about 74%, at least or about 75%, at least or about 76%, at least or about 77%, at least or about 78%, at least or about 79%, at least or about 80%, at least or about 81%, at least or about 82%, at least or about 83%, at least or about 84%, at least or about 85%, at least or about 86%, at least or about 87%, at least or about 88%, at least or about 89%, at least or about 90%, at least or about 91%, at least or about 92%, at least or about 93%, at least or about 94%, at least or about 95%, at least or about 96%, at least or about 97%, at least or about 98%, at least or about 99%, or about 100%, identical to the amino acid sequence set forth in SEQ ID NO: 11, SEQ ID NO: 12, SEQ ID NO: 26, SEQ ID NO: 27, SEQ ID NO: 28, SEQ ID NO: 29, or SEQ ID NO: 30.

TABLE 1

| | | SEQ |
|---|---|---|
| | | ID |
| Name | Sequence | NO. |
| SARS-CoV-2 E protein | MYSFVSEETGTLIVNSVL | 1 |
| N-terminal 1-18 amino | | |
| acid residues (aa) | | |
| MY18-2ED | MYSFVSDDTGTLIVNSVL | 2 |
| TAT-PEG3-MY18-2ED | GRKKRRQRRRPPQXMYSFVSDDTGTLIVNSVL | 3 |
| | [X is 13-atom polyethylene glycol linker; or X | |
| | is (PEG)3.] | |
| TAT | GRKKRRQRRRPPQ | 4 |
| 6-Arg (6Arg) | RRRRRR | 5 |
| Penetratin (Pen) | RQIKIWFQNRRMKWKK | 6 |
| TAT-MY18 | GRKKRRQRRRPPQMYSFVSEETGTLIVNSVL | 7 |
| TAT-MY18-2ED | GRKKRRQRRRPPQMYSFVSDDTGTLIVNSVL | 8 |
| 6-Arg (Arg)-MY18 | RRRRRRMYSFVSEETGTLIVNSVL | 9 |
| Penetratin (Pen)-MY18 | RQIKIWFQNRRMKWKKMYSFVSEETGTLIVNSV | 10 |
| | L | |
| SARS-CoV-1 E protein | MYSFVSEETGTLIVNSVLLFLAFVVFLLVTLAIL | 11 |
| (1-76 aa) | TALRLCAYCCNIVNVSLVKPTVYVYSRVKNLNS | |
| | SEGVPDLLV | |
| SARS-CoV-2 E protein | MYSFVSEETGTLIVNSVLLFLAFVVFLLVTLAIL | 12 |
| (1-75 aa) | TALRLCAYCCNIVNVSLVKPSFYVYSRVKNLNS | |
| | SRVPDLLV | |
| SARS-CoV-2 E protein | MYSFVSEETGTLIVNSVLLFLA | 13 |
| N-terminal 1-22 aa | | |
| MERS-CoV E protein N- | MLPFVQERIGLFIVNFFI | 14 |
| terminal 1-18 aa | | |
| MERS-COV MY18 (ED | MLPFVQDRIGLFIVNFFI | 15 |
| or DE) | | |
| MERS-CoV MY18 R8H | MLPFVQEHIGLFIVNFFI | 16 |
| HCoV-229E E protein N- | MFLKLVDDHALVVNVLLW | 17 |
| terminal 1-18 aa | | |
| HCOV-229E MY18 (ED | MFLKLVEEHALVVNVLLW | 18 |
| or DE) | | |
| HCoV-NL63 E protein | MFLRLIDDNGIVLNSILW | 19 |
| N-terminal 1-18 aa | | |
| HCoV-NL63 MY18 (ED | MFLRLIEENGIVLNSILW | 20 |
| or DE) | | |
| HCoV-NL63 MY18 2DE | MFLRLIEEDGIVLNSILW | 21 |
| & N9D | | |
| HCoV-OC43 E protein | MADAYLADTVWYVGQIIF | 22 |
| N-terminal 3-20 aa | | |
| HCoV-OC43 MY18 (ED | MADAYLAETVWYVGQIIF | 23 |
| or DE) | | |
| HCoV-HKU1 E protein | MVDLFENDTAWYIGQILV | 24 |
| N-terminal 1-18 aa | | |
| HCoV-HKU1 MY18 | VDLFFNETAWYIGQILV | 25 |
| (ED or DE) | | |
| MERS-CoV E protein | MLPFVQERIGLFIVNFFIFTVVCAITLLVCMAFLT | 26 |
| sequence | ATRLCVQCMTGFNTLLVQPALYLYNTGRSVYV | |
| | KFQDSKPPLPPDEWV | |
| HCoV-OC43 E protein | MFMADAYLADTVWYVGQIIFIVAICLLVTIVVV | 27 |
| sequence | AFLATFKLCIQLCGMCNTLVLSPSIYVFNRGRQF | |
| | YEFYNDVKPPVLDVDDV | |
| HCoV-HKU1 E protein | MVDLFFNDTAWYIGQILVLVLFCLISLIFVVAFL | 28 |
| sequence | ATIKLCMQLCGFCNFFIISPSAYVYKRGMQLYKS | |
| | YSEQVIPPTSDYLI | |
| HCoV-229E E protein | MFLKLVDDHALVVNVLLWCVVLIVILLVCITIIK | 29 |
| sequence | LIKLCFTCHMFCNRTVYGPIKNVYHIYQSYMHI | |
| | DPFPKRVIDF | |
| HCoV-NL63 E protein | MFLRLIDDNGIVLNSILWLLVMIFFFVLAMTFIK | 30 |
| sequence | LIQLCFTCHYFFSRTLYQPVYKIFLAYQDYMQIA | |
| | PVPAEVLNV | |
| SARS-CoV-2 qPCR N | CTCTTGTAGATCTGTTCTCTAAACGAAC | 31 |
| forward primer | | |
| SARS-CoV-2 qPCR N | GGTCCACCAAACGTAATGCG | 32 |
| reverse primer | | |
| SARS-CoV-2 qPCR E | CTCATTCGTTTCGGAAGAGACAG | 33 |
| forward primer | | |
| SARS-CoV-2 qPCR E | AGACCAGAAGATCAGGAACTCTAG | 34 |
| reverse primer | | |
| Mouse Gapdh qPCR | CTTCACCACCATGGAGAAGG | 35 |
| forward primer | | |
| Mouse Gapdh qPCR | TGAAGTCGCAGGAGACAACC | 36 |
| reverse primer | | |
| Monkey qPCR GAPDH | GAAGGTGAAGGTCGGAGTCAAC | 37 |
| (for Vero-E6) forward | | |
| primer | | |
| Monkey qPCR GAPDH | TCGTTGTCATACCAGGAAATGAGC | 38 |
| (for Vero-E6) reverse | | |
| primer | | |
| Human NFATC4 qPCR | CTTCTCCGATGCCTCTGACG | 39 |
| forward primer | | |
| Human NFATC4 qPCR | CGGGGCTTGGACCATACAG | 40 |
| reverse primer | | |
| Human JUN/AP-1 qPCR | ACTCGGACCTTCTCACGTC | 41 |
| forward primer | | |
| Human JUN/AP-1 qPCR | GGTCGGTGTAGTGGTGATGT | 42 |
| reverse primer | | |
| Human GAPDH qPCR | CTCACCGGATGCACCAATGTT | 43 |
| forward primer | | |
| Human GAPDH qPCR | CGCGTTGCTCACAATGTTCAT | 44 |
| reverse primer | | |

The coronavirus peptide may have about 6 to about 70 amino acid residues in length, about 8 to about 65 amino acid residues in length, about 10 to about 60 amino acid residues in length, about 10 to about 55 amino acid residues in length, about 10 to about 50 amino acid residues in length, about 10 to about 45 amino acid residues in length, about 10 to about 40 amino acid residues in length, about 10 to about 35 amino acid residues in length, about 10 to about 30 amino acid residues in length, about 10 to about 20 amino acid residues in length, about 12 to about 55 amino acid residues in length, about 12 to about 50 amino acid residues in length, about 12 to about 45 amino acid residues in length, about 12 to about 40 amino acid residues in length, about 12 to about 35 amino acid residues in length, about 12 to about 30 amino acid residues in length, about 12 to about 25 amino acid residues in length, about 12 to about 20 amino acid residues in length, about 15 to about 55 amino acid residues in length, about 15 to about 50 amino acid residues in length, about 15 to about 45 amino acid residues in length, about 15 to about 40 amino acid residues in length, about 15 to about 35 amino acid residues in length, about 15 to about 30 amino acid residues in length, about 15 to about 25 amino acid residues in length, about 15 to about 20 amino acid residues in length, about 6 amino acid residues in length, about 7 amino acid residues in length, about 8 amino acid residues in length, about 9 amino acid residues in length, about 10 amino acid residues in length, about 11 amino acid residues in length, about 12 amino acid residues in length, about 13 amino acid residues in length, about 14 amino acid residues in length, about 15 amino acid residues in length, about 16 amino acid residues in length, about 17 amino acid residues in length, about 19 amino acid residues in length, about 20 amino acid residues in length, about 21 amino acid residues in length, about 22 amino acid residues in length, about 23 amino acid residues in length, about 24 amino acid residues in length, about 25 amino acid residues in length, or about 18 amino acid residues in length, where the coronavirus peptide has an amino acid sequence at least or about 50%, at least or about 55%, at least or about 60%, at least or about 61%, at least or about 62%, at least or about 63%, at least or about 64%, at least or about 65%, at least or about 66%, at least or about 67%, at least or about 68%, at least or about 69%, at least or about 70%, at least or about 71%, at least or about 72%, at least or about 73%, at least or about 74%, at least or about 75%, at least or about 76%, at least or about 77%, at least or about 78%, at least or about 79%, at least or about 80%, at least or about 81%, at least or about 82%, at least or about 83%, at least or about 84%, at least or about 85%, at least or about 86%, at least or about 87%, at least or about 88%, at least or about 89%, at least or about 90%, at least or about 91%, at least or about 92%, at least or about 93%, at least or about 94%, at least or about 95%, at least or about 96%, at least or about 97%, at least or about 98%, at least or about 99%, or about 100%, identical to a consecutive amino acid sequence, within position 1 to position 75, within position 1 to position 70, within position 1 to position 65, within position 1 to position 60, within position 1 to position 55, within position 1 to position 50, within position 1 to position 45, within position 1 to position 40, within position 1 to position 35, within position 1 to position 30, within position 1 to position 25, within position 1 to position 24, within position 1 to position 23, within position 1 to position 22, within position 1 to position 21, within position 1 to position 20, within position 1 to position 19, within position 1 to position 18, within position 2 to position 40, within position 2 to position 35, within position 2 to position 30, within position 3 to position 20, or within position 2 to position 25, of the wildtype envelope (E) protein of a coronavirus.

The coronavirus peptide may be greater than 50 amino acid residues in length, between about 5 and about 50 amino acid residues in length, between about 5 and about 45 amino acid residues in length, between about 5 and about 40 amino acid residues in length, between about 5 and about 35 amino acid residues in length, between about 5 and about 30 amino acid residues in length, between about 5 and about 25 amino acid residues in length, between about 5 and about 20 amino acid residues in length, between about 8 and about 20 amino acid residues in length, between about 10 and about 20 amino acid residues in length, or between about 15 and about 20 amino acid residues in length. In certain embodiments, the coronavirus peptide is 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, or more than 40, amino acid residues in length.

In certain embodiments, the coronavirus peptide comprises at least one glutamic acid (Glu, or E) to aspartic acid (Asp, or D) mutation, and/or at least one aspartic acid to glutamic acid mutation. In certain embodiments, 1, 2, 3, 4, 5, 6, or all of glutamic acid (Glu, or E) of the coronavirus peptide are substituted with aspartic acid (Asp, or D). In certain embodiments, 1, 2, 3, 4, 5, 6, or all of aspartic acid (Asp, or D) of the coronavirus peptide are substituted with glutamic acid (Glu, or E).

In certain embodiments, the coronavirus peptide may comprise (or consist essentially of, or consist of) an amino acid sequence at least or about 50%, at least or about 55%, at least or about 60%, at least or about 61%, at least or about 62%, at least or about 63%, at least or about 64%, at least or about 65%, at least or about 66%, at least or about 67%, at least or about 68%, at least or about 69%, at least or about 70%, at least or about 71%, at least or about 72%, at least or about 73%, at least or about 74%, at least or about 75%, at least or about 76%, at least or about 77%, at least or about 78%, at least or about 79%, at least or about 80%, at least or about 81%, at least or about 82%, at least or about 83%, at least or about 84%, at least or about 85%, at least or about 86%, at least or about 87%, at least or about 88%, at least or about 89%, at least or about 90%, at least or about 91%, at least or about 92%, at least or about 93%, at least or about 94%, at least or about 95%, at least or about 96%, at least or about 97%, at least or about 98%, at least or about 99%, or about 100%, identical to the amino acid sequence set forth in SEQ ID NO: 1, SEQ ID NO: 13, SEQ ID NO: 14, SEQ ID NO: 17, SEQ ID NO: 19, SEQ ID NO: 22 or SEQ ID NO: 24, or fragments, variants, analogs, orthologs, homologs or derivatives thereof.

In certain embodiments, the coronavirus peptide may comprise (or consist essentially of, or consist of) an amino acid sequence at least or about 50%, at least or about 55%, at least or about 60%, at least or about 61%, at least or about 62%, at least or about 63%, at least or about 64%, at least or about 65%, at least or about 66%, at least or about 67%, at least or about 68%, at least or about 69%, at least or about 70%, at least or about 71%, at least or about 72%, at least or about 73%, at least or about 74%, at least or about 75%, at least or about 76%, at least or about 77%, at least or about 78%, at least or about 79%, at least or about 80%, at least or about 81%, at least or about 82%, at least or about 83%, at least or about 84%, at least or about 85%, at least or about 86%, at least or about 87%, at least or about 88%, at least or about 89%, at least or about 90%, at least or about 91%, at least or about 92%, at least or about 93%, at least or about 94%, at least or about 95%, at least or about 96%, at least or about 97%, at least or about 98%, at least or about 99%, or about 100%, identical to the amino acid sequence set forth in SEQ ID NO: 2, SEQ ID NO: 15, SEQ ID NO: 16, SEQ ID NO: 18, SEQ ID NO: 20, SEQ ID NO: 21, SEQ ID NO: 23 or SEQ ID NO: 25, or fragments, variants, analogs, orthologs, homologs or derivatives thereof.

In certain embodiments, the present fusion polypeptide comprises a cell-penetrating peptide.

Cell-penetrating peptides (CPPs) can cross the cellular membrane. In certain embodiments, cell-penetrating peptides gain entry into the cell via endocytosis and/or direct translocation through the cellular membrane. Endocytosis occurs by various mechanisms, including clathrin-dependent endocytosis and clathrin-independent endocytosis.

Cell-penetrating peptides can be cationic, amphipathic, hydrophobic, anionic, hydrophilic, or non-amphipathic. In certain embodiments, the present fusion polypeptide comprises a non-cationic cell-penetrating peptide.

Cell-penetrating peptides can be linear or cyclical. Cell-penetrating peptides can be random coiled, alpha-helical, or contain beta-sheets.

In certain embodiments, the cell-penetrating peptide has up to 60, up to 50, up to 40, up to 30, up to 20, up to 18, up to 15, up to 12, up to 11, or up to 10, amino acid residues in length. In certain embodiments, the cell-penetrating peptide has about 10-20, 12-20, 12-16, 13-16, 6-20, 6-16, 6-13, 8-10, 8-12, 8-15, 8-20, 8-30, 8-40, 8-50, or 8-60 amino acid residues in length. In certain embodiments, the cell-penetrating peptide has about 7-10, 7-12, 7-15, 7-20, 7-30, 7-40, 7-50, or 7-60 amino acid residues in length.

The cell-penetrating peptide may be TAT, 6-Arg or Penetratin.

In certain embodiments, the cell-penetrating peptide may comprise (or consist essentially of, or consist of) an amino acid sequence at least or about 50%, at least or about 55%, at least or about 60%, at least or about 61%, at least or about 62%, at least or about 63%, at least or about 64%, at least or about 65%, at least or about 66%, at least or about 67%, at least or about 68%, at least or about 69%, at least or about 70%, at least or about 71%, at least or about 72%, at least or about 73%, at least or about 74%, at least or about 75%, at least or about 76%, at least or about 77%, at least or about 78%, at least or about 79%, at least or about 80%, at least or about 81%, at least or about 82%, at least or about 83%, at least or about 84%, at least or about 85%, at least or about 86%, at least or about 87%, at least or about 88%, at least or about 89%, at least or about 90%, at least or about 91%, at least or about 92%, at least or about 93%, at least or about 94%, at least or about 95%, at least or about 96%, at least or about 97%, at least or about 98%, at least or about 99%, or about 100%, identical to the amino acid sequence set forth in SEQ ID NO: 4, SEQ ID NO: 5, or SEQ ID NO: 6, or fragments, variants, analogs, orthologs, homologs or derivatives thereof.

Non-limiting examples of cell-penetrating peptides also include the cell-penetrating peptides in Tables 1-6. Milletti, F., Cell-penetrating peptides: classes, origin, and current landscape, Drug Discovery Today, 2012, Volume 17, Numbers 15/16: 850-860, the content of which is incorporated herein by reference in its entirety.

In certain embodiments, the cell-penetrating peptide may comprise (or consist essentially of, or consist of) an amino acid sequence at least or about 50%, at least or about 55%, at least or about 60%, at least or about 61%, at least or about 62%, at least or about 63%, at least or about 64%, at least or about 65%, at least or about 66%, at least or about 67%, at least or about 68%, at least or about 69%, at least or about 70%, at least or about 71%, at least or about 72%, at least or about 73%, at least or about 74%, at least or about 75%, at least or about 76%, at least or about 77%, at least or about 78%, at least or about 79%, at least or about 80%, at least or about 81%, at least or about 82%, at least or about 83%, at least or about 84%, at least or about 85%, at least or about 86%, at least or about 87%, at least or about 88%, at least or about 89%, at least or about 90%, at least or about 91%, at least or about 92%, at least or about 93%, at least or about 94%, at least or about 95%, at least or about 96%, at least or about 97%, at least or about 98%, at least or about 99%, or about 100%, identical to the amino acid sequence set forth in any of SEQ ID NOS: 45-148, or fragments, variants, analogs, orthologs, homologs or derivatives thereof.

TABLE 2
CPPs derived from heparan-, RNA- and DNA-binding proteins
Cationic
Heparan binding proteins

| | | |
|---|---|---|
| RKKRRRESRKKRRRES | DPV3 | SEQ ID NO: 45 |
| GRPRESGKKRKRKRLKP | DPV6 | SEQ ID NO: 46 |
| GKRKKKGKLGKKRDP | DPV7 | SEQ ID NO: 47 |
| GKRKKKGKLGKKRPRSR | DPV7b | SEQ ID NO: 48 |
| RKKRRRESRRARRSPRHL | DPV3/10 | SEQ ID NO: 49 |
| SRRARRSPRESGKKRKRKR | DPV10/6 | SEQ ID NO: 50 |
| VKRGLKLRHVRPRVTRMDV | DPV1047 | SEQ ID NO: 51 |
| SRRARRSPRHLGSG | DPV10 | SEQ ID NO: 52 |
| LRRERQSRLRRERQSR | DPV15 | SEQ ID NO: 53 |
| GAYDLRRRERQSRLRRRERQSR | DPV15b | SEQ ID NO: 54 |

RNA binding proteins

| | | |
|---|---|---|
| RKKRRQRRR | HIV-1 Tat | SEQ ID NO: 55 |
| RRRRNRTRRNRRRVR | FHV coat | SEQ ID NO: 56 |
| TRQARRNRRRRWRERQR | HIV-1 Rev | SEQ ID NO: 57 |
| TRRQRTRRARRNR | HTLV-II Rex | SEQ ID NO: 58 |
| KMTRAQRRAAARRNRWTAR | BMV Gag | SEQ ID NO: 59 |
| NAKTRRHERRRKLAIER | P22 N | SEQ ID NO: 60 |
| MDAQTRRRERRAEKQAQWKAAN | λN(1-22) | SEQ ID NO: 61 |
| TAKTRYKARRAELIAERR | φ21N(12-29) | SEQ ID NO: 62 |
| TRRNKRNRIQEQLNRK | Yeast PrP6 | SEQ ID NO: 63 |

DNA binding proteins

| | | |
|---|---|---|
| PRRRRSSSRPVRRRRRPRVSRRRRRRGGRRRR | Protamine 1 | SEQ ID NO: 64 |
| Leucine zipper | | |
| RIKAERKRMRNRIAASKSRKRKLERIAR | Human cJun | SEQ ID NO: 65 |
| KRRIRRERNKMAAAKSRNRRRELTDT | Human cFos | SEQ ID NO: 66 |

Transcription factors

| | | |
|---|---|---|
| KRARNTEAARRSRARKLQRMKQ | Yeast GCN4 | SEQ ID NO: 67 |

Homeoproteins

| | | |
|---|---|---|
| RQIKIWFQNRRMKWKK | Penetratin | SEQ ID NO: 68 |
| RVIRVWFQNKRCKDKK | Islet-1 | SEQ ID NO: 69 |
| SKRTRQTYTRYQTLELEKEFHENRYITRRRRIDI | Fushi-tarazu | SEQ ID NO: 70 |
| ANALSLSERQIKIWFQNRRMKSKKDR | | |
| SQIKIWFQNKRAKIKK | Engrailed-2 | SEQ ID NO: 71 |
| RQVTIWFQNRRVKEKK | HoxA-13 | SEQ ID NO: 72 |
| KQINNWFINQRKRHWK | Knotted-1 | SEQ ID NO: 73 |
| RHIKIWFQNRRMKWKK | PDX-1 | SEQ ID NO: 74 |

TABLE 3
CPPs derived from signal peptides
Amphipathic (I): signal peptide + NLS

| | | |
|---|---|---|
| MGLGLHLLVLAAALQGAKKKRKV | Ig(v) | SEQ ID NO: 75 |
| MVKSKIGSWILVLFVAMWSDVGLCKKRPKP | BPrPp(1-30) | SEQ ID NO: 76 |
| MANLGYWLLALFVTMWTDVGLCKKRPKP | MPrPp(1-28) | SEQ ID NO: 77 |
| AAVLLPVLLAAPVQRKRQKLP | K-FGF + NLS | SEQ ID NO: 78 |

Hydrophobic: signal peptide alone

| | | |
|---|---|---|
| AAVLLPVLLAAP | K-FGF | SEQ ID NO: 79 |

TABLE 4
CPPs derived from antimicrobial peptides
Pro-rich

| | | |
|---|---|---|
| RRIRPRPPRLPRPRPRPLPFPRPG | Bac7 | SEQ ID NO: 80 |
| VDKGSYLPRPTPPRPIYNRN | Pyrrhocoricin | SEQ ID NO: 81 |

Amphipathic

| | | |
|---|---|---|
| KCFQWQRNMRKVRGPPVSCIKR | Human lactoferrin | SEQ ID NO: 82 |
| | (19-40) | |
| TRSSRAGLQWPVGRVHRLLRK | Buforin 2 | SEQ ID NO: 83 |
| GIGAVLKVLTTGLPALISWIKRKRQQ | Melittin | SEQ ID NO: 84 |
| GIGKWLHSAKKFGKAFVGEIMNS | Magainin 2 | SEQ ID NO: 85 |
| LLGDFFRKSKEKIGKEFKRIVQRIK- | LL-37 | SEQ ID NO: 86 |
| DFLRNLVPRTESC | | |
| RGGRLSYSRRRFSTSTGR | SynB1 | SEQ ID NO: 87 |
| YKQCHKKGGKKGSG | Crotamine | SEQ ID NO: 88 |
| ALWKTLLKKVLKAPKKKRKV | S4₁₃-PVᵣₑᵥ | SEQ ID NO: 89 |
| HARIKPTFRRLKWKYKGKFW | L-2 | SEQ ID NO: 90 |

TABLE 5
CPPs derived from viral proteins
Unknown structure

| | | |
|---|---|---|
| TKRRITPKDVIDVRSVTTEINT | Inv3 | SEQ ID NO: 91 |

Amphipathic

| | | |
|---|---|---|
| RQGAARVTSWLGRQLRIAGKRLEGRSK | Erns | SEQ ID NO: 92 |
| NAATATRGRSAASRPTQRPRAPARSASRPR | VP22 | SEQ ID NO: 93 |
| RPVQ | | |
| RHSRIGIIQQRRTRNG | HIV-1 VPR | SEQ ID NO: 94 |
| | 77-92 | |
| KLIKGRTPIKFGKADCDRPPKHSQNGMGK | Ribotoxin2 L3 | SEQ ID NO: 95 |
| | loop | |
| PLSSIFSRIGDP | PreS2-TLM | SEQ ID NO: 96 |

Amphipathic (b-sheet)

| | | |
|---|---|---|
| DPKGDPKGVTVTVTVTVTGKGDPKPD | VT5 | SEQ ID NO: 97 |

TABLE 6
CPPs derived from various natural proteins
Cationic

| | | |
|---|---|---|
| RRIPNRRPRR | HRSV | SEQ ID NO: 98 |
| RLRWR | AIP6 | SEQ ID NO: 99 |

Amphipathic (I)

| | | |
|---|---|---|
| MVRRFLVTLRIRRACGPPRVRV | ARF(1-22) | SEQ ID NO: 100 |
| MVTVLFRRLRIRRACGPPRVRV | M918 | SEQ ID NO: 101 |
| LLIILRRRIRKQAHAHSK | pVEC | SEQ ID NO: 102 |

Amphipathic (helical)

| | | |
|---|---|---|
| LSTAADMQGVVTDGMASG | Azurin p18 | SEQ ID NO: 103 |
| LSTAADMQGVVTDGMASGLDKDYLKPDD | Azurin p28 | SEQ ID NO: 104 |
| KFHTFPQTAIGVGAP | hCT18-32 | SEQ ID NO: 105 |

Hydrophobic

| | | |
|---|---|---|
| VPTLK (PMLKE, VPALR, VSALK, | Bip | SEQ ID NO: 106 |
| IPALK) | | (SEQ ID NO: |
| | | 145, SEQ ID |
| | | NO: 146, SEQ |
| | | ID NO: 147, |
| | | SEQ ID NO: |
| | | 148) |
| PFVYLI | C105Y | SEQ ID NO: 107 |
| PIEVCMYREP | FGF12 | SEQ ID NO: 108 |

TABLE 7
Designed CPPs and CPPs derived from peptide libraries
Designed
Cationic

| | |
|---|---|
| R8, R9, R10, R12 | Polyarginine |

Amphipathic (cationic I)

| | | |
|---|---|---|
| KETWWETWWTEWSQPKKRKV | Pep-1 | SEQ ID NO: 109 |
| GLAFLGFLGAAGSTMGAWSQPKKKRKV | MPG | SEQ ID NO: 110 |

Amphipathic (cationic II)

| | | |
|---|---|---|
| GWTLNSAGYLLGKINLKALAALAKKIL | Transportan | SEQ ID NO: 111 |
| AGYLLGHINLHHLAHLAibHHIL | TH | SEQ ID NO: 112 |
| KLALKALKALKAALKLA | MAP | SEQ ID NO: 113 |
| RRWWRRWRR | W/R | SEQ ID NO: 114 |
| GLWRALWRLLRSLWRLLWRA | CADY | SEQ ID NO: 115 |
| LIRLWSHLIHIWFQNRRLKWKKK | EB-1 | SEQ ID NO: 116 |

Amphipathic (anionic II)

| | | |
|---|---|---|
| WEAALAEALAEALAEHLAEALAEALEALAA | GALA | SEQ ID NO: 117 |
| LKTLTETLKELTKTLTEL | MAP12 | SEQ ID NO: 118 |

Amphipathic (zero-charge II)

| | | |
|---|---|---|
| QLALQLALQALQAALQLA | MAP17 | SEQ ID NO: 119 |

Amphipathic (Proline-rich)

| | | |
|---|---|---|
| (PPR)3, (PPR)4, (PPR)5, (PPR)6 | (PPR)n | |
| (PRR)3, (PRR)4, (PRR)5, (PRR)6 | (PRR)n | |
| GPSQPTYPGDDAPVRDLIRFYRDLQRYLNVVTR | aPP4R1 | SEQ ID NO: 120 |
| HRY | | |
| GPSQPTYPGDDAPVRDLIRFYRDLRRYLNVVTR | aPP5R1 | SEQ ID NO: 121 |
| HRY | | |
| GPSQPTYPGDDAPVRDLRRFYRDLRRYLNVVTR | aPP6R1 | SEQ ID NO: 122 |
| HRY | | |
| G(P_{L}XX)_{N}P_{I} | PoliProline- | |
| | based | |
| VRLPPPVRLPPPVRLPPP | SAP | SEQ ID NO: 123 |
| VELPPPVELPPPVELPPP | SAP(E) | SEQ ID NO: 124 |

Peptide libraries
Support-vector machine model

| | | |
|---|---|---|
| FKIYDKKVRTRVVKH | SVM1 | SEQ ID NO: 125 |
| RASKRDGSWVKKLHRILE | SVM2 | SEQ ID NO: 126 |
| KGTYKKKLMRIPLKGT | SVM3 | SEQ ID NO: 127 |
| LYKKGPAKKGRPPLRGWFH | SVM4 | SEQ ID NO: 128 |
| HSPIIPLGTRFVCHGVT | SVM5 | SEQ ID NO: 129 |
| YTAIAWVKAFIRKLRK | YTA2 | SEQ ID NO: 130 |
| IAWVKAFIRKLRKGPLG | YTA4 | SEQ ID NO: 131 |
| IAWVKAFIRKLRKGPLG | YTA4 | SEQ ID NO: 132 |

Plasmid display
Amphipathic

| | | |
|---|---|---|
| RLSGMNEVLSFRWL | SG3 | SEQ ID NO: 133 |

Phage display
Hydrophobic

| | | |
|---|---|---|
| SDLWEMMMVSLACQY | Pep-7 | SEQ ID NO: 134 |
| VTWTPQAWFQWV | | SEQ ID NO: 135 |
| GSPWGLQHHPPRT | 439a | SEQ ID NO: 136 |
| GPFHFYQFLFPPV | 435b | SEQ ID NO: 137 |
| TSPLNIHNGQKL | HN-1 | SEQ ID NO: 138 |

Other

| | |
|---|---|
| CAYHRLRRC | SEQ ID NO: 139 |

Phylomer library
Cationic

| | | |
|---|---|---|
| RCGRASRCRVRWMRRRRI | BEN_1079 | SEQ ID NO: 140 |

Other

| | | |
|---|---|---|
| PYSRPHVQLWYPNRESCRSLIRSLGP | BEN_0805 | SEQ ID NO: 141 |

Peptide arrays
Hydrophobic

| | | |
|---|---|---|
| PLILLRLLRGQF | Pept1 | SEQ ID NO: 142 |
| PLIYLRLLRGQF | Pept2 | SEQ ID NO: 143 |
| KLWMRWYSPTTRRYG | IVV-14 | SEQ ID NO: 144 |

The cell-penetrating peptide may be directly linked to the coronavirus peptide (e.g., without use of a linker).

Alternatively, the cell-penetrating peptide may be linked to the coronavirus peptide via a linker.

In one embodiment, the linker may be non-immunogenic in the subject.

The linker may be a peptide linker or non-peptide linker.

For peptide linkers, such linker sequences may be a naturally occurring sequence or a non-naturally occurring sequence.

An exemplary non-peptide linker is a PEG linker.

“PEG,” “polyethylene glycol” and “poly(ethylene glycol)” as used herein, are interchangeable and encompass any nonpeptidic, water-soluble polyethylene oxide (PEO). In certain embodiments, PEGs may comprise the following structure “—(OCH₂CH₂)n—” where n is 1, 2, 3, 4, 5, 6, 7, 8, 9, 10, 11, 12, 13, 14, 15, 16, 17, 18, 19, 20, or up to 4000. As used herein, PEG also includes “—CH₂CH₂—O(CH₂CH₂O)n—CH₂CH₂—” and“—(OCH₂CH₂)nO—” where n is 1, 2, 3, 4, 5, 6, 7, 8, 9, 10, 11, 12, 13, 14, 15, 16, 17, 18, 19, 20, or up to 4000, depending upon whether or not the terminal oxygens have been displaced, e.g., during a synthetic transformation. The term “PEG” includes structures having various terminal or “end capping” groups and so forth. The term “PEG” also means a polymer that contains a majority, that is to say, greater than 50%, of —OCH₂CH₂— repeating subunits. With respect to specific forms, the PEG can take any number of a variety of molecular weights, as well as structures or geometries such as “branched,” “linear,” “forked,” “multifunctional,” and the like, to be described in greater detail below.

PEG is commercially available or can be prepared by ring-opening polymerization of ethylene glycol according to methods well known in the art (Sandler and Karo, Polymer Synthesis, Academic Press, New York, Vol. 3, pages 138-161). In the present application, the term “PEG” may encompass any polyethylene glycol molecule, in mono-, bi-, or poly-functional form, without regard to size or to modification of the PEG. PEG may be represented by the formula: —O(CH₂CH₂O)ₙ₋₁CH₂CH₂OH, where n is 1, 2, 3, 4, 5, 6, 7, 8, 9, 10, 11, 12, 13, 14, 15, 16, 17, 18, 19, 20, or up to 4000.

In certain embodiments, the PEG covalently linked to the present polypeptide/peptide has no greater than 60 atoms. In certain embodiments, the PEG covalently linked to the present polypeptide/peptide is monodispersed PEG.

In certain embodiments, the PEG linker of the present polypeptide/peptide may comprise (or consist essentially of, or consist of) PEG, (PEG)2, (PEG)3, (PEG)4, (PEG)5, (PEG)6, (PEG)7, (PEG)8, (PEG)9, (PEG)10, (PEG)11, (PEG)12, (PEG)13, (PEG)14, (PEG)15, or PEG containing more than 15 monomer units.

In one embodiment, the PEG linker of the present polypeptide/peptide, or the PEG covalently linked to the present polypeptide/peptide, is PEG (or monoethylene glycol). In one embodiment, the PEG linker of the present polypeptide/peptide, or the PEG covalently linked to the present polypeptide/peptide, is (PEG)2 (or diethylene glycol, or 8-amino-3,6-dioxaoctanoic acid). In one embodiment, the PEG linker of the present polypeptide/peptide, or the PEG covalently linked to the present polypeptide/peptide, is (PEG)3 (or triethylene glycol, or 12-amino-4,7,10-trioxadodecanoic acid). In one embodiment, the PEG linker of the present polypeptide/peptide, or the PEG covalently linked to the present polypeptide/peptide, is (PEG)4 (or tetraethylene glycol, or 15-amino-4,7,10,13-tetraoxapenta-decanoic acid).

In certain embodiments, the PEG linker of the present polypeptide/peptide may comprise (or consist essentially of, or consist of) a 7-atom polyethylene glycol linker, 8-atom polyethylene glycol linker, 9-atom polyethylene glycol linker, 13-atom polyethylene glycol linker, 14-atom polyethylene glycol linker, 16-atom polyethylene glycol linker, 18-atom polyethylene glycol linker, 20-atom polyethylene glycol linker, 24-atom polyethylene glycol linker, 30-atom polyethylene glycol linker, or 31-atom polyethylene glycol linker.

In certain embodiments, the PEG covalently linked to the present polypeptide/peptide has greater than 60 atoms. In certain embodiments, the PEG covalently linked to the present polypeptide/peptide is polydispersed PEG.

In one embodiment, the PEG groups may be attached to the polypeptide or peptide via acylation or reductive alkylation (or reductive amination) through a reactive group on the PEG moiety (e.g., an aldehyde, amino, thiol, or ester group) and to a reactive group on the polypeptide or peptide (e.g., an aldehyde, amino, or ester group). A strategy for the PEGylation of peptides includes combining, through forming a conjugate linkage in solution, a peptide and a PEG moiety, each bearing a special functionality that is mutually reactive toward the other. The peptides can be prepared with conventional solid phase synthesis. The peptides are “preactivated” with an appropriate functional group at a specific site. The precursors are purified and fully characterized prior to reacting with the PEG moiety. Ligation of the peptide with PEG may take place in aqueous phase and can be monitored by reverse phase analytical HPLC. The PEGylated peptides can be purified by preparative HPLC and characterized by analytical HPLC, amino acid analysis and laser desorption mass spectrometry.

Covalent conjugation of proteins and peptides with polyethylene glycol (PEG) may or may not extend the in vivo circulating half-lives of the polypeptide or peptide. Additional properties that may or may not be conferred by PEGylation include increased solubility, resistance to proteolytic degradation, and reduced immunogenicity of the polypeptide or peptide.

In some embodiments, a peptidyl linker is present (i.e., made up of amino acids linked together by peptide bonds) that is made in length, e.g., of from 1 to about 40 amino acid residues, from 1 to about 20 amino acid residues, and from 1 to about 10 amino acid residues. In certain embodiments, the amino acid residues in the linker are from among cysteine, glycine, alanine, proline, asparagine, glutamine, and/or serine. In certain embodiments, a peptidyl linker is made up of a majority of amino acids that are sterically unhindered, such as glycine, serine, and alanine. Linkers also include polyglycines (e.g., (Gly)4, (Gly)5), poly(Gly-Ala), and polyalanines.

The disclosure also provides for derivatives of the polypeptide or peptide. Such derivatives may be obtained by modification, e.g., by chemical and/or genetic modification, of the polypeptide or peptide and/or of one or more of the amino acid residues that form the polypeptide or peptide.

For example, such a modification may involve the introduction (e.g., by covalent linking or in another suitable manner) of one or more functional groups, residues or moieties into or onto the polypeptide/peptide, and in particular of one or more functional groups, residues or moieties that confer one or more desired properties or functionalities to the polypeptide/peptide.

Moieties that can be covalently attached to the present peptide/polypeptide include, but are not limited to: PEG, copolymers of ethylene glycol and propylene glycol, carboxymethyl cellulose, dextran, polyvinyl alcohol, polyvinyl pyrrolidone and polyproline. See, for example, Abuchowski and Davis (1981), Soluble Polymer-Enzyme Adducts, Enzymes as Drugs (Hocenberg and Roberts, eds.), Wiley-Interscience, New York, N.Y., pp 367-83; Newmark, et al. (1982), J. Appl. Biochem. 4:185-9. Other polymers that could be used are poly-1,3-dioxolane and poly-1,3,6-tioxocane.

The present polypeptide/peptide may be modified by moieties including, but not limited to, a copolymer of ethylene glycol, a copolymer of propylene glycol, a carboxymethylcellulose, a polyvinyl pyrrolidone, a poly-1,3-dioxolane, a poly-1,3,6-trioxane, an ethylene/maleic anhydride copolymer, a polyaminoacid (e.g., polylysine), a dextran n-vinyl pyrrolidone, a poly n-vinyl pyrrolidone, a propylene glycol homopolymer, a propylene oxide polymer, an ethylene oxide polymer, a polyoxyethylated polyol, a polyvinyl alcohol, a linear or branched glycosylated chain, a polyacetal, a long chain fatty acid, a long chain hydrophobic aliphatic group, an albumin (e.g., human serum albumin (HSA)); a transthyretin (TTR), or a thyroxine-binding globulin (TBG).

For example, such modification may comprise the introduction (e.g., by covalent binding or in any other suitable manner) of one or more functional groups that increase the half-life, the solubility and/or the absorption of the polypeptide/peptide, that reduce the immunogenicity and/or the toxicity of the polypeptide/peptide, that eliminate or attenuate any undesirable side effects of the polypeptide/peptide, and/or that confer other advantageous properties to and/or reduce the undesired properties of the polypeptide/peptide; or any combination of two or more of the foregoing.

One of the techniques for increasing the half-life and/or reducing the immunogenicity of proteins, polypeptides or peptides comprises attachment of a suitable pharmacologically acceptable polymer, such as polyethylene glycol (PEG) or derivatives thereof (such as methoxypoly(ethyleneglycol) or mPEG). Generally, any suitable form of PEGylation can be used; reference is made to, for example, Chapman, Nat. Biotechnol., 54, 531-545 (2002); Veronese and Harris, Adv. Drug Deliv. Rev. 54, 453-456 (2003); Harris and Chess, Nat. Rev. Drug. Discov., 2, (2003) and WO 04/060965. Various reagents for PEGylation of proteins are also commercially available. In one embodiment, site-directed PEGylation is used, e.g., via a cysteine-residue (see for example Yang et al., Protein Engineering, 16, 10, 761-770 (2003).

By “PEGylated” is meant a peptide, polypeptide or protein having a polyethylene glycol (PEG) moiety covalently bound to one or more amino acid residues of the peptide/polypeptide/protein itself or to a peptidyl or non-peptidyl linker (including but not limited to aromatic or aryl linkers) that is covalently bound to one or more amino acid residues of the peptide/polypeptide/protein.

The peptide/polypeptide may be modified by a polyalkylene glycol compound (such as polyethylene glycol) or a derivative thereof, with or without coupling agents or derivatization with coupling or activating moieties (e.g., with aldehyde, hydroxysuccinimidyl, hydrazide, thiol, triflate, tresylate, azirdine, oxirane, orthopyridyl disulphide, vinylsulfone, iodoacetamide or a maleimide moiety).

Any molecular mass (molecular weight) for PEG may be used, e.g., from about 50 Daltons (Da) to about 5,000 Da, from about 100 Da to about 2,000 Da, from about 100 Da to about 1,000 Da, from about 100 Da to about 500 Da, from about 100 Da to about 300 Da, from about 1,000 or 2,000 Da to about 100,000 Da or higher, from about 3,000 Da or 5,000 Da, to about 50,000 Da or 60,000 Da, from about 10,000 Da to about 40,000 Da, or from about 20,000 Da to about 30,000 Da. The PEG may have a molecular weight (or an average molecular weight) of about 100 to about 5000 kDa, about 100 to about 500 kDa, from about 100 Daltons to about 150,000 Daltons, greater than 5,000 Daltons to about 100,000 Daltons, from about 6,000 Daltons to about 90,000 Daltons, from about 10,000 Daltons to about 85,000 Daltons, from about 10,000 Daltons to about 85,000 Daltons, from about 20,000 Daltons to about 85,000 Daltons, from about 53,000 Daltons to about 85,000 Daltons, from about 25,000 Daltons to about 120,000 Daltons, from about 29,000 Daltons to about 120,000 Daltons, from about 35,000 Daltons to about 120,000 Daltons, or from about 40,000 Daltons to about 120,000 Daltons.

In some embodiments, a PEG used to modify the present polypeptide/peptide terminates on one end with hydroxy or methoxy, i.e., X is H or CH3 (“methoxy PEG”).

PEGylation may include site-specific PEGylation at any suitable amino acid residue(s) on the polypeptide/peptide. PEG may be a linker linking two amino acid residues of the polypeptide/peptide. The polypeptide/peptide may have N-terminal PEGylation, internal PEGylation, and/or C-terminal PEGylation.

The present polypeptide/peptide may be modified by a biologically suitable polymer or copolymer, for example, a polyalkylene glycol compound, such as a polyethylene glycol or a polypropylene glycol. Other appropriate polyalkylene glycol compounds include, but are not limited to, charged or neutral polymers of the following types: dextran, polylysine, colominic acids or other carbohydrate based polymers, polymers of amino acids, and biotin derivatives.

Polysaccharide polymers are another type of water-soluble polymer that can be used for polypeptide/peptide modification. Dextrans are polysaccharide polymers comprised of individual subunits of glucose predominantly linked by α1-6 linkages. The dextran itself is available in many molecular weight ranges, and may include dextran in molecular weights from about 1 kDa to about 70 kDa, or about 1 kDa to about 20 kDa.

Another modification comprises N-linked or O-linked glycosylation, usually as part of co-translational and/or post-translational modification, depending on the host cell used for expressing the polypeptide/peptide.

Another modification may comprise the introduction of a functional group that is one part of a specific binding pair, such as the biotin-(strept)avidin binding pair. Such a functional group may be used to link the polypeptide/peptide to another protein, polypeptide or chemical compound that is bound to the other half of the binding pair, i.e., through formation of the binding pair. For example, a polypeptide/peptide may be conjugated to biotin, and linked to another protein, polypeptide, compound or carrier conjugated to avidin or streptavidin. Additionally, a conjugated polypeptide/peptide may be used as a reporter, for example in a diagnostic system where a detectable signal-producing agent is conjugated to avidin or streptavidin. Such binding pairs may for example also be used to bind the polypeptide/peptide to a carrier, including carriers suitable for pharmaceutical purposes.

Other potential chemical and enzymatical modifications are known in the art. Such modifications may also be introduced for research.

The polypeptides or peptides disclosed herein can be formulated in compositions, including pharmaceutical compositions. Such compositions can be formulated in such a way to deliver the polypeptides or peptides to the target tissue or cell.

Such compositions or pharmaceutical compositions can comprise more than one polypeptide/peptide disclosed herein. Such compositions could comprise two, three, four, five or more polypeptides/peptides.

The pharmaceutical compositions may comprise a nucleic acid encoding the polypeptides or peptides and a pharmaceutically acceptable carrier.

The phrase “pharmaceutically acceptable” as used herein refers to molecular entities and compositions that are physiologically tolerable and do not typically produce an allergic or similar untoward reaction, such as gastric upset, dizziness and the like, when administered to a human, and approved by a regulatory agency of the Federal or a state government or listed in the U.S. Pharmacopeia or other generally recognized pharmacopeia for use in animals, and more particularly in humans.

The term “carrier” refers to a diluent, adjuvant, excipient, or vehicle with which the therapeutic is administered. Such pharmaceutical carriers can be sterile liquids, such as saline solutions in water and oils, including those of petroleum, animal, vegetable, or synthetic origin, such as peanut oil, soybean oil, mineral oil, sesame oil, and the like. A saline solution is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol, and the like. The composition, if desired, can also contain minor amounts of wetting or emulsifying agents, or pH buffering agents. Adjuvants can also be added to the RNA to protect it from degradation.

Compositions, polypeptides and nucleic acids according to the present disclosure may be administered to a subject by conventional routes, such as intravenously, orally, delivered to the nose (nasal administration), upper respiratory tract and/or lung, sublingually, parenterally, or topically. The compositions and pharmaceutical composition may be formulated such that the polypeptides or nucleic acids according to the present disclosure reach the target tissue or cells.

The present polypeptides/peptides, nucleic acids, or pharmaceutical compositions may be delivered by nasal administration, or pulmonary administration (e.g., by nebulization). Pharmaceutical compositions adapted for nasal and pulmonary administration may comprise solid carriers such as powders, which can be administered by rapid inhalation through the nose. Compositions for nasal administration may comprise liquid carriers, such as sprays or drops. The present polypeptides/peptides or pharmaceutical compositions may be delivered by inhalation. Inhalation into the lungs may be accomplished by inhalation deeply or installation through a mouthpiece. These compositions may comprise aqueous or oil solutions of the active ingredient. Compositions for inhalation may be supplied in specially adapted devices including, but not limited to, pressurized aerosols, nebulizers or insufflators, which can be constructed so as to provide predetermined dosages of the polypeptides/peptides.

The present polypeptides/peptides or pharmaceutical compositions may be administered by nebulization. This route may provide a high local concentration in the airways and lungs to ensure rapid onset of therapeutic effects, while limiting the potential for unwanted systemic effects.

The present polypeptides/peptides, nucleic acids, or pharmaceutical compositions may be delivered by parenteral administration. Pharmaceutical compositions adapted for parenteral administration, including intravenous administration, may contain aqueous and non-aqueous sterile injectable solutions or suspensions, which may include anti-oxidants, buffers, bacteriostats, and solutes that render the compositions substantially isotonic with the blood of the subject. Other components which may be present in such compositions include water, alcohols, polyols, glycerin, and vegetable oils. Compositions adapted for parental administration may be presented in unit-dose or multi-dose containers, such as sealed ampules and vials, and may be stored in a freeze-dried (lyophilized) condition requiring only the addition of a sterile carrier, immediately prior to use. Extemporaneous injection solutions and suspensions may be prepared from sterile powders, granules, and tablets. Suitable vehicles that can be used to provide parenteral dosage forms of the invention are well known to those skilled in the art. Examples include: Water for Injection USP; aqueous vehicles such as Sodium Chloride Injection, Ringer's Injection, Dextrose Injection, Dextrose and Sodium Chloride Injection, and Lactated Ringer's Injection; water-miscible vehicles such as ethyl alcohol, polyethylene glycol, and polypropylene glycol; and non-aqueous vehicles such as corn oil, cottonseed oil, peanut oil, sesame oil, ethyl oleate, isopropyl myristate, and benzyl benzoate.

The present polypeptides/peptides, nucleic acids, or pharmaceutical compositions may be delivered by oral administration. Pharmaceutical compositions adapted for oral administration may be capsules, tablets, powders, granules, solutions, syrups, suspensions (in non-aqueous or aqueous liquids), or emulsions. Tablets or hard gelatin capsules may comprise lactose, starch or derivatives thereof, magnesium stearate, sodium saccharine, cellulose, magnesium carbonate, stearic acid or salts thereof. Soft gelatin capsules may comprise vegetable oils, waxes, fats, semi-solid, or liquid polyols. Solutions and syrups may comprise water, polyols, and sugars. An active agent intended for oral administration may be coated with or admixed with a material that delays disintegration and/or absorption of the active agent in the gastrointestinal tract. Thus, the sustained release may be achieved over many hours and if necessary, the active agent can be protected from degradation within the stomach. Pharmaceutical compositions for oral administration may be formulated to facilitate release of an active agent at a particular gastrointestinal location due to specific pH or enzymatic conditions.

Further routes of administration of the present polypeptides/peptides, nucleic acids, or pharmaceutical compositions include sublingual, vaginal, buccal, or rectal; or transdermal administration to a subject.

Selection of a therapeutically effective dose will be determined by the skilled artisan considering several factors, which will be known to one of ordinary skill in the art. Such factors include the particular form of the pharmacological agent, and its pharmacokinetic parameters such as bioavailability, metabolism, and half-life, which will have been established during the usual development procedures typically employed in obtaining regulatory approval for a pharmaceutical compound. Further factors in considering the dose include the condition or disease to be treated or the benefit to be achieved in a normal individual, the body mass of the patient, the route of administration, whether the administration is acute or chronic, concomitant medications, and other factors well known to affect the efficacy of administered pharmaceutical agents. Thus, the precise dose should be decided according to the judgment of the person of skill in the art, and each patient's circumstances, and according to standard clinical techniques.

The present disclosure provides for a method of treating, preventing and/or alleviating the symptoms of an infection or disease as well as prevention from severe illness caused by a coronavirus (e.g., a human coronavirus such as SARS-CoV-2). The method may comprise administering to a subject in need thereof an effective amount of the present polypeptide/peptide, or the pharmaceutical composition.

In some embodiments, the composition comprises more than one polypeptide/peptide. In some embodiments, the composition comprises two polypeptides/peptides. In some embodiments, the composition comprises three polypeptides/peptides. In some embodiments, the composition comprises four polypeptides/peptides. In some embodiments, the composition comprises five or more polypeptides/peptides.

The present disclosure provides for a method of treating, preventing and/or alleviating the symptoms of an infection or disease as well as prevention from severe illness caused by a coronavirus (e.g., a human coronavirus such as SARS-CoV-2). The method may comprise administering to a subject in need thereof an effective amount of a nucleic acid encoding the present polypeptide/peptide.

A further embodiment is a use of the present polypeptide/peptide, a nucleic acid encoding the polypeptide/peptide, or the present pharmaceutical composition, for the preparation of a medicament for treating, preventing and/or alleviating the symptoms of an infection or disease caused by a coronavirus (e.g., a human coronavirus such as SARS-CoV-2).

The present disclosure provides for kits for practicing any of the methods disclosed herein, including methods of treating, preventing and/or alleviating the symptoms of an infection or disease caused by a coronavirus (e.g., a human coronavirus such as SARS-CoV-2).

The present disclosure provides for kits which may include the present polypeptide/peptide, a nucleic acid encoding the present polypeptide/peptide, or the pharmaceutical composition.

The present kits may further include containers for suitable administration and a package insert including information concerning the pharmaceutical compositions and dosage forms in the kit. Generally, such information aids patients and physicians in using the enclosed pharmaceutical compositions and dosage forms effectively and safely. For example, the following information regarding a combination of the invention may be supplied in the insert: pharmacokinetics, pharmacodynamics, clinical studies, efficacy parameters, indications and usage, contraindications, warnings, precautions, adverse reactions, overdosage, proper dosage and administration, how supplied, proper storage conditions, references, manufacturer/distributor information and patent information.

There may be numerous tools and techniques within the skill of the art, such as those commonly used in molecular immunology, cellular immunology, pharmacology, and microbiology. See, e.g., Sambrook et al. (2001) Molecular Cloning: A Laboratory Manual. 3rd ed. Cold Spring Harbor Laboratory Press: Cold Spring Harbor, N.Y.; Ausubel et al. eds. (2005) Current Protocols in Molecular Biology, John Wiley and Sons, Inc.: Hoboken, N.J.; Bonifacino et al. eds. (2005) Current Protocols in Cell Biology, John Wiley and Sons, Inc.: Hoboken, N.J.; Coligan et al. eds. (2005) Current Protocols in Immunology, John Wiley and Sons, Inc.: Hoboken, N.J.; Coico et al. eds. (2005) Current Protocols in Microbiology, John Wiley and Sons, Inc.: Hoboken, N.J.; Coligan et al. eds. (2005) Current Protocols in Protein Science, John Wiley and Sons, Inc.: Hoboken, N.J.; and Enna et al. eds. (2005) Current Protocols in Pharmacology, John Wiley and Sons, Inc.: Hoboken, N.J.

The singular forms “a,” “an,” and “the” include plural reference unless the context clearly dictates otherwise.

The term “about” is used herein to mean approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 10%.

The term “polypeptide” and “peptide” are used interchangeably herein.

The term “subject” as used in this application means an animal such as avians and mammals. Mammals include canines, felines, rodents, bovine, equines, porcines, ovines, and primates. Avians include, but are not limited to, fowls, songbirds, and raptors. Thus, the polypeptide/peptide, pharmaceutical composition, or kit may be used in veterinary medicine, e.g., to treat companion animals, farm animals, laboratory animals in zoological parks, and animals in the wild. In one embodiment, the subject is a human.

In certain embodiments, the term “subject” is meant a patient or a human subject. In some embodiments, the subject is one suffering with an infection or disease caused by a coronavirus (e.g., a human coronavirus such as SARS-CoV-2) or is suspected of suffering from an infection or disease caused by a coronavirus (e.g., a human coronavirus such as SARS-CoV-2), or at risk from an infection or disease caused by a coronavirus (e.g., a human coronavirus such as SARS-CoV-2), or has been exposed to a coronavirus (e.g., a human coronavirus such as SARS-CoV-2). In some embodiments, the patient has not been exposed to a coronavirus (e.g., a human coronavirus such as SARS-CoV-2).

The terms “treat”, “treatment”, and the like refer to a means to slow down, relieve, ameliorate or alleviate at least one of the symptoms of the disease, or reverse the disease after its onset.

The terms “prevent”, “prevention”, and the like refer to acting prior to overt disease or disorder onset, to prevent the disease or disorder from developing or minimize the extent of the disease or disorder or slow its course of development.

The present polypeptide/peptide, a nucleic acid encoding the present polypeptide/peptide, or the pharmaceutical composition, or the present method, may be used to treat a subject in need thereof. The term “in need thereof” would be a subject known or suspected of having or being at risk of an infection or disease caused by a coronavirus (e.g., a human coronavirus such as SARS-CoV-2) or has been exposed to a coronavirus (e.g., a human coronavirus such as SARS-CoV-2). In some embodiments, the subject has not been exposed to a coronavirus (e.g., a human coronavirus such as SARS-CoV-2).

The terms “therapeutically effective amount” or “effective amount” encompasses an amount sufficient to ameliorate or prevent a symptom or sign of the medical condition. Effective amount also means an amount sufficient to allow or facilitate diagnosis. An effective amount for a particular subject may vary depending on factors such as the condition being treated, the overall health of the patient, the method route and dose of administration and the severity of side effects. An effective amount can be the maximal dose or dosing protocol that avoids significant side effects or toxic effects.

The following are examples of the present invention and are not to be construed as limiting.

### Example 1

We report an inhibitory peptide against SARS-CoV-2 E named iPep-SARS2-E. Taking advantage of E-induced alternations in proton homeostasis and NFAT/AP-1 pathway in mammalian cells, we developed screening platforms to optimize our peptides. The experiments using SARS-CoV-2 virus reveal that iPep-SARS2-E significantly inhibits E and virus egress and reduces viral cytotoxicity and propagation in vitro and in vivo. Furthermore, the peptide can be customizable for other human coronaviruses such as MERS-CoV. The results indicate that E can be a potential therapeutic target for human coronaviruses.

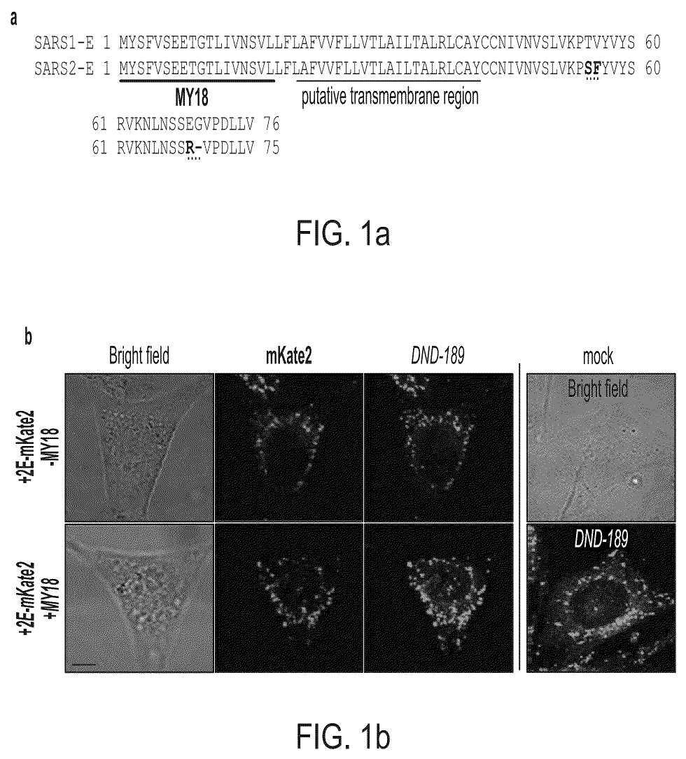

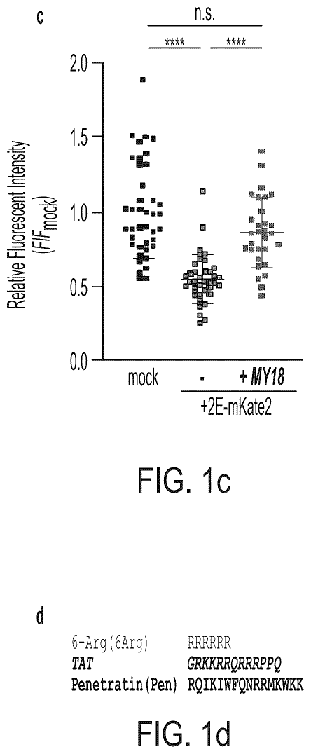

SARS-CoV-2 E (2E) protein has 75 amino-acid residues, high homology with SARS-CoV-1 E protein (˜96%) with identical transmembrane and pore structures^{10,16,17} (FIG. 1a). Previous studies reported that deficiency of SARS-CoV-1 E gene significantly reduced viral propagation¹¹, suggesting that 2E may also play essential roles in viral function and can be a potential therapeutic target for COVID-19 and future variants. Because 2E induces cellular toxicity in a number of different ways^{11,15,18-20}, in this study we seek to develop screening platforms to identify drug candidates against 2E. In addition, we examined whether our approach to 2E can be applicable for the other human coronavirus E.

### Results

Because 2E demonstrates high homology with SARS-CoV-1 E protein with identical transmembrane and pore structures (FIG. 1a), we first produced a monoclonal antibody against the amino (N)-terminal region. Following previous studies of viral envelope topology, we believed that this may be in the extracellular region of SARS-CoV-2 virus and as such was a suitable therapeutic target^{16,21}. To produce the monoclonal antibody, we used a synthetic peptide composed of the first 18 amino acids and Keyhole Limpet haemocyanin (KLH) as the antigen. Western blotting results reveal that a hybridoma produces a 2E monoclonal antibody (2E-N; clone, N2A5E8) that can recognize 2E proteins expressed in a mammalian heterologous expression system. However, immunocytochemistry using the antibody demonstrates that it could not detect all of the 2E-YFP proteins expressed in mammalian cells. These results suggest that the antibody may bind to only the denatured and/or monomeric form of 2E proteins in mammalian cells since we used a synthetic peptide containing the first 18 amino acids as the antigen. To address this concern, we expressed 2E construct fused to a His tag in bacteria, purified the recombinant protein using Ni resin and then incubated the protein in a 2E-N affinity column, which is composed of Protein G Agarose cross-linked to the 2E-N monoclonal antibody. We found that the affinity column could not purify the recombinant protein. These results suggest that the N-terminus might be integrated to the protein complex and not exposed to the outside as the previous studies reported in SARS-CoV-1 E and 2E oligomers^{10,13,16,17,22}. Therefore, the antibody 2E-N that we produced may not be applied to functional tests such as cytopathic assays using live SARS-CoV-2 virus because the antibody may not bind to the surface of SARS-CoV-2 virus where oligomerized 2E complexes are expressed.

Following the results from the 2E monoclonal antibody, we hypothesized that the N-terminal fragment might be able to disrupt 2E protein function because the N-terminus could be integrated into the protein complex. Because our previous study demonstrates that the overexpression of 2E affects proton homeostasis in intracellular organelles such as Golgi apparatus and lysosomes in mammalian cells¹⁹, we examined the effect of the N-terminal fragment named MY18 (18 amino acids, MYSFVSEETGTLIVNSVL) on 2E function using DND-189 pH fluorescent dye and MY18 plasmid transfection in mammalian cells. DND-189-based pH fluorescent imaging shows that MY18 co-overexpression significantly restores the proton homeostasis in 2E-expressing mammalian cells (FIG. 1b, FIG. 1c). These results suggest that MY18 can be used as an inhibitory peptide that disrupts 2E function. Next, to apply MY18 as a synthetic peptide, we sought cell-penetrating amino acid motifs and tested three candidates: an arginine repeat, TAT and Penetratin²³ (FIG. 1d). The DND-189-based pH imaging demonstrates that the TAT version of MY18 is an effective cell-penetrating peptide (FIG. 1e).

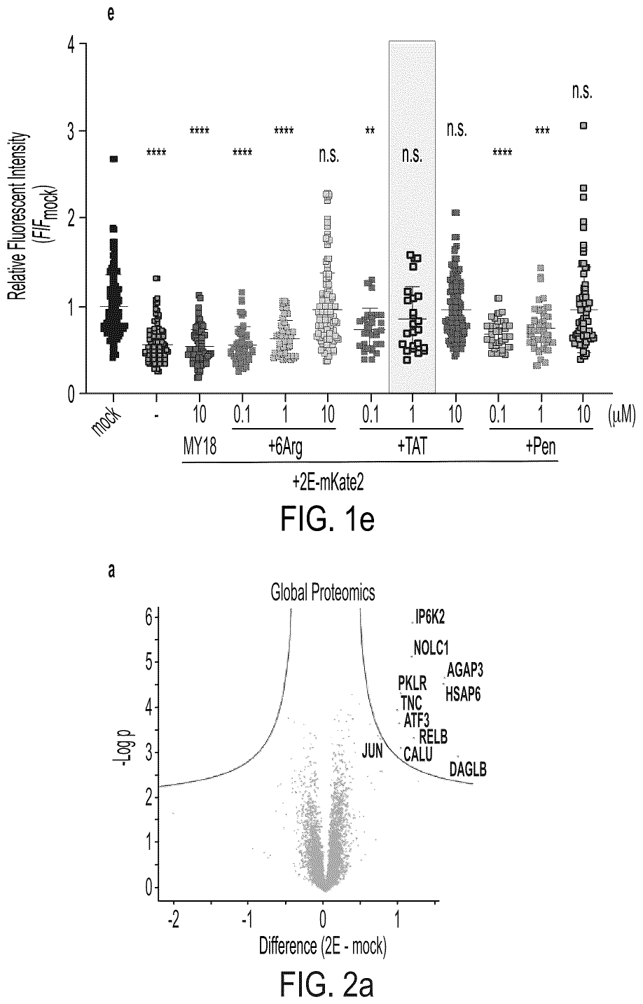

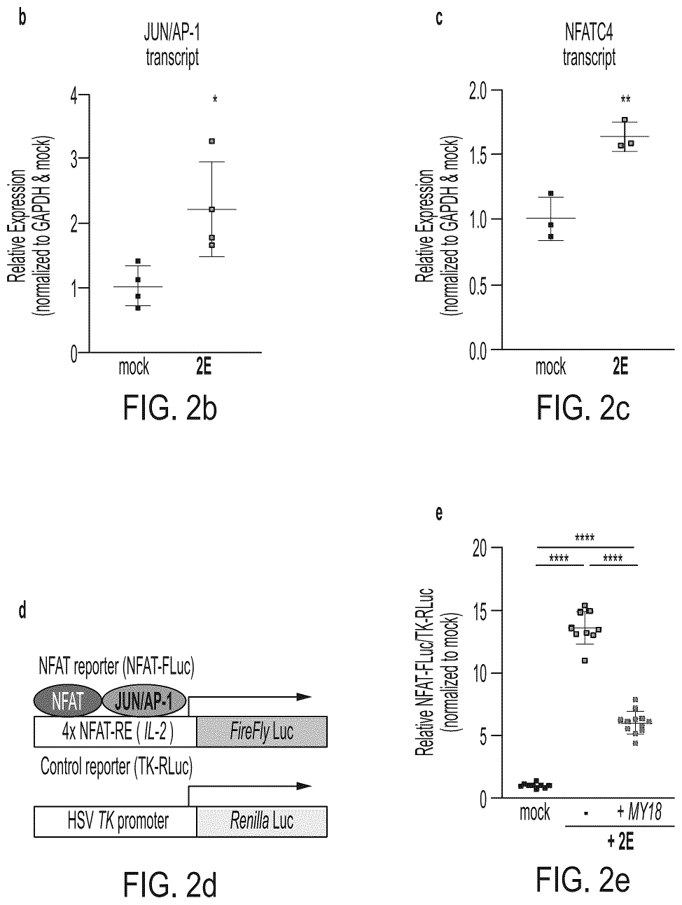

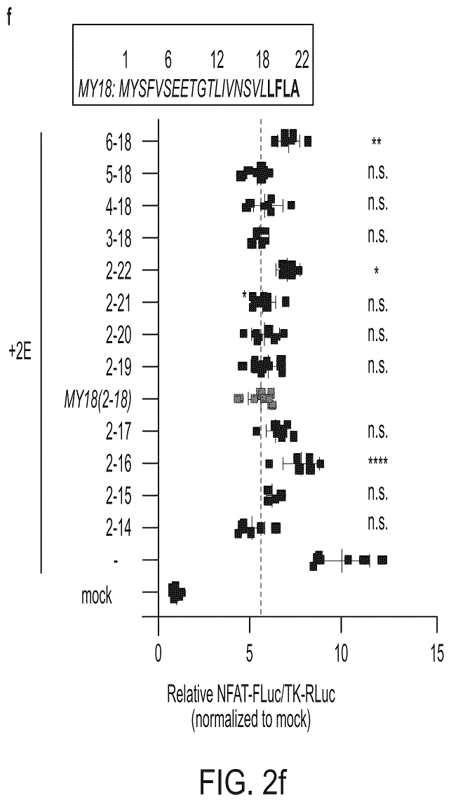

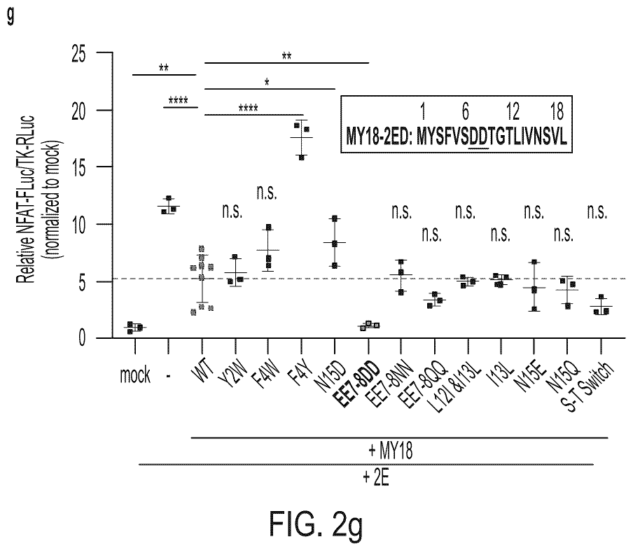

While DND-189-based pH fluorescent imaging is useful as a drug screening platform of live mammalian cells, the dynamic range of the dye is somewhat limited, the standard deviation of fluorescent readout is relatively large, and its throughput is not so high as an assay. These limitations might result in difficulty for optimizing the MY18 peptide further using molecular biological approaches with mutagenesis. To develop a higher-throughput screening platform, we overexpressed 2E in mammalian cells and explored other reliable and quantitative readouts. Interestingly, the global proteomics result demonstrates various increases of key signaling molecules such as JUN/AP-1 (FIG. 2a). Our follow-up experiment using quantitative RT-PCR confirms that the expression of JUN transcript is significantly increased in 2E-expressing cells compared to mock (FIG. 2b). Also, the transcript expression of a related molecule, NFATC4/NFAT3, is also upregulated significantly (FIG. 2c) though the increase of NFATC4/NFAT3 protein (˜20%) did not reach to significance in statistical analysis of the global proteomics results with the standard false discovery rate (FDR, 0.05, FIG. 2a). Following these results, we decided to apply the NFAT response element of the human IL-2 gene where NFAT and JUN/AP-1 interact²⁴. To obtain precise readouts, we used the dual luciferase reporter system containing NFAT Firefly luciferase reporter and pRL-TK-Renilla luciferase reporter as the transfection control using HSV TK, herpes simplex virus thymidine kinase, promoter²⁵ (FIG. 2d). The luciferase assay result obtained using plasmid DNA co-transfection shows that 2E overexpression significantly increases the Firefly luciferase activity in mammalian cells and that MY18 significantly suppresses the effect of 2E on the NFAT/AP-1 pathway though it does not fully reach to the level of mock (FIG. 2e). These results suggest that MY 18 is not sufficient to prevent 2E from altering the NFAT/AP-1 pathway completely though we observed that MY18 restores proton homeostasis in DND-189-based pH fluorescent imaging. Looking to optimize MY18, we examined whether deletion or extension of MY18 might improve the effect on interrupting 2E function using the luciferase reporter. We found that none of the constructs significantly improved the effect as the majority reduced efficacy (FIG. 2f). Next, using mutagenesis, transfection and luciferase assay, we tested a variety of MY18 mutant constructs and found that the substitution of glutamate to asparagine at 7^{th} and 8^{th} residues (EE7-8DD, 2ED) significantly improved MY18 (FIG. 2g). We combined the mutant 2ED and TAT cell-penetrating motif (FIG. 1d, FIG. 1e) and called TAT-MY18-2ED “iPep-SARS-2E” (inhibitory Peptide against SARS-CoV-2 Envelope).

To characterize iPep-SARS2-E, we first conducted an ELISA to compare the affinity of our 2E-N antibody to our peptides, 2ED and wild-type. We confirmed that the 2ED mutation reduces the binding affinity of the 2E antibody, which was produced using the wild-type MY18 peptide. Next, we used ELISA to examine how stable iPep-SARS2-E is in phosphate buffered saline (PBS) at 37° C. We did not observe obvious peptide degradation in 24 hours while the peptide might become unstable after 48 hours because the standard deviation becomes larger compared to the other time points. Using an apoptosis/necrosis assay with flow cytometry, we confirmed that iPep-SARS-2E peptide does not have cellular toxicity in mammalian cells in vitro.

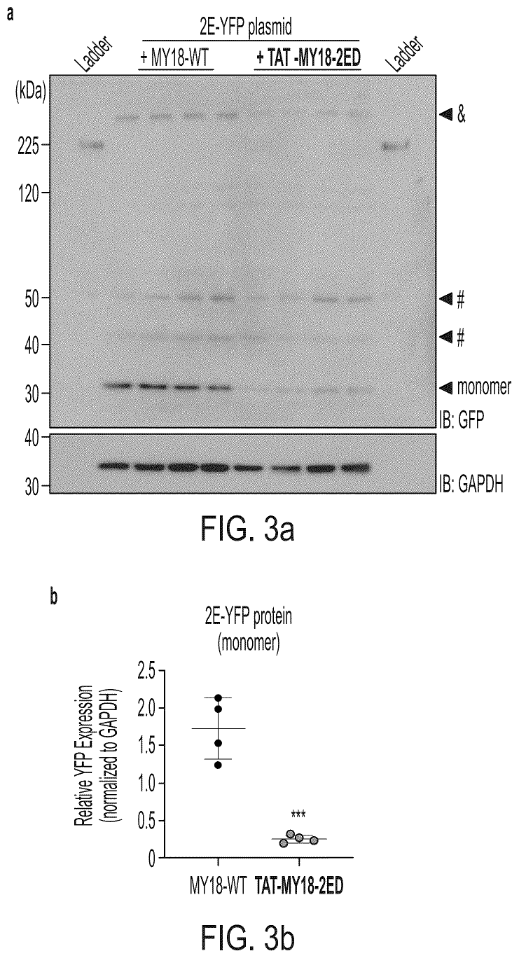

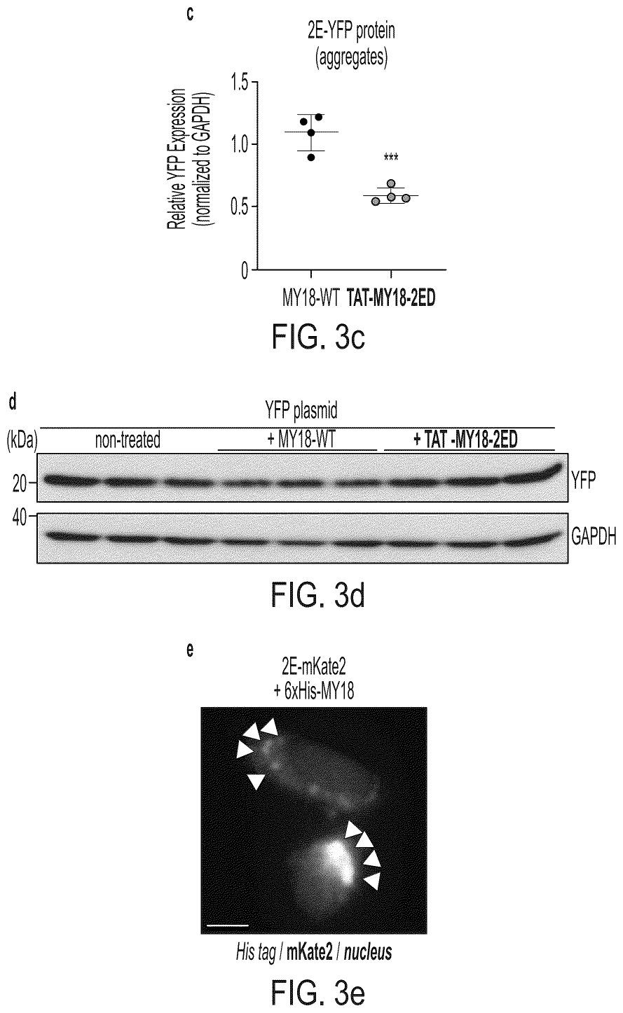

To investigate the molecular mechanism underlying the inhibitory effect of iPep-SARS2-E, we incubated mammalian cells with iPep-SARS2-E and transfected them with 2E-YFP construct to examine the effect of iPep-SARS2-E on 2E protein expression. Interestingly, iPep-SARS2-E significantly reduced 2E-YFP protein expression in mammalian cells. While we observed both monomeric and aggregate bands of 2E proteins, both forms were significantly decreased in the treated cells (FIGS. 3a-3c). As a control experiment, we used YFP transfected cells with peptide treatment and did not observe any effect of iPep-SARS2-E on YFP protein expression (FIG. 3d). The results indicate that iPep-SARS2-E does not affect lipofection but reduces 2E expression in mammalian cells potentially through a dominant-negative effect. Next, we co-expressed 2E-mKate2 with MY18 peptide fused to a His tag in NIH 3T3 cells using lipofection and anti-His tag antibody to examine whether MY18 peptide interacts 2E protein directly. The imaging result reveals that MY18 peptides are co-localized with 2E proteins (FIG. 3e), suggesting that MY18 peptide might be integrated into 2E protein complex and inactivate 2E and restore lysosomal activity. 2E may induce deacidification in lysosome. MY18 peptides may be integrated into 2E protein complexes, resulting in 2E inactivation and restored lysosomal activity and 2E protein reduction.

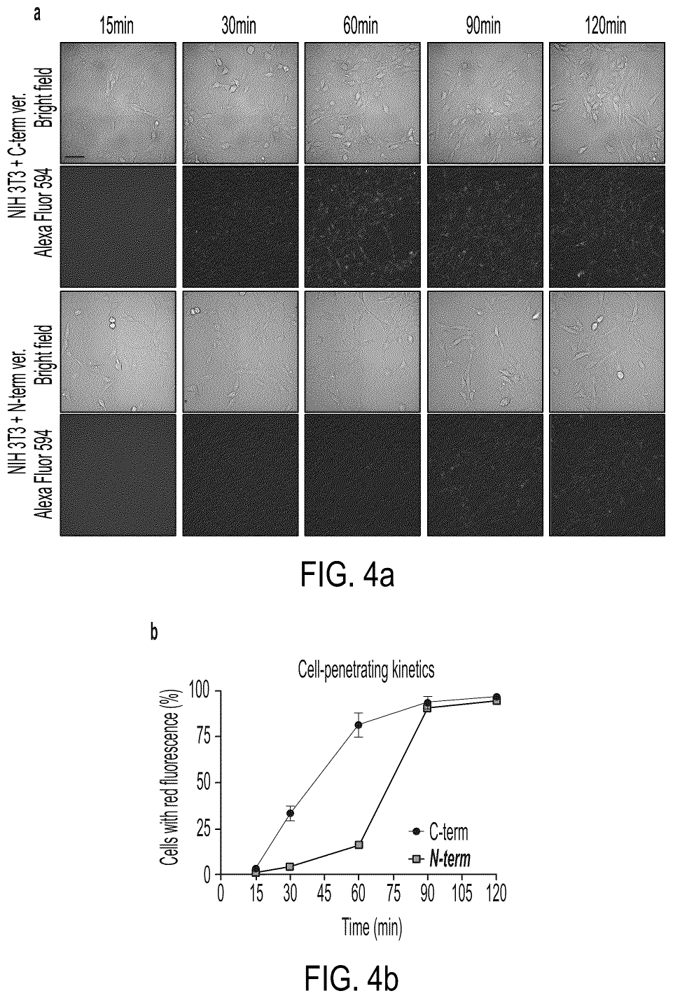

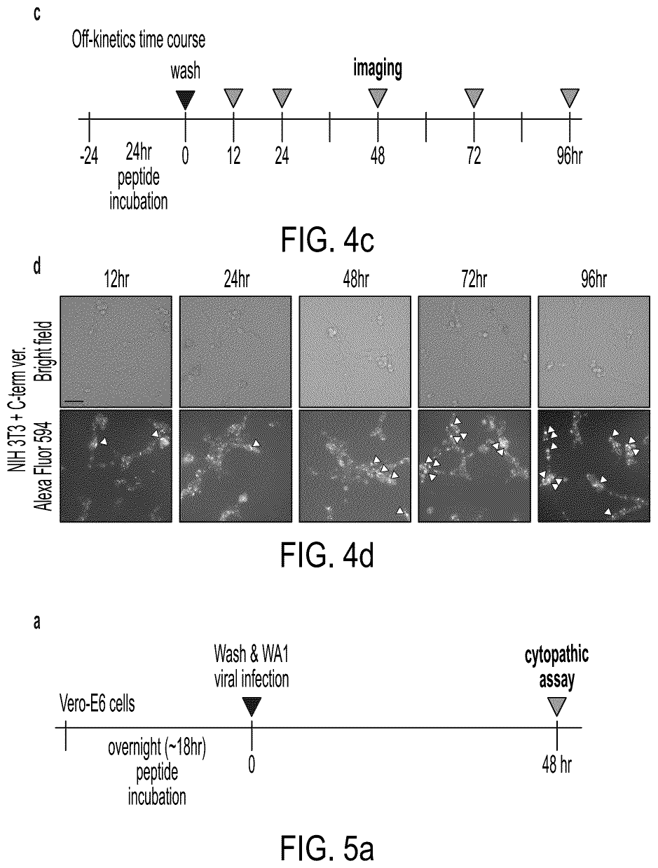

To further characterize iPep-SARS2-E in situ, we conjugated a fluorescent probe, Alexa Fluor 594, to the N- or carboxyl (C)-terminus of iPep-SARS2-E to examine the kinetics of peptide penetration into mammalian cells. The fluorescent imaging in situ reveals that the C-terminal fused version has faster cell-penetrating than N-terminal version and most cells can uptake the peptides in 2 hours (FIG. 4a, FIG. 4b). This result suggests that the N-terminal conjugation of Alexa Fluor 594 might affect the function of the TAT motif in the peptide. To examine the off kinetics, we next treated cells with iPep-SARS2-E fluorescent peptide (C-terminal version, TAT-MY18-2ED-Alexa Fluor 594) for 24 hours, washed out the culture medium, and then monitored the fluorescence (FIG. 4c). The in situ imaging suggests that the peptide is not reduced/degraded for 96 hours though the peptide might become unstable and aggregated after 72 hours at 37° C. in the culture medium since larger fluorescent puncta were observed compared to earlier time points (FIG. 4d). In addition, we tested the C-terminal version in another mammalian cell line Vero-E6, which has been commonly used in viral studies as well as for our cytopathic assay using live SARS-CoV-2 virus. We confirmed that the peptide can penetrate into these cells as well.

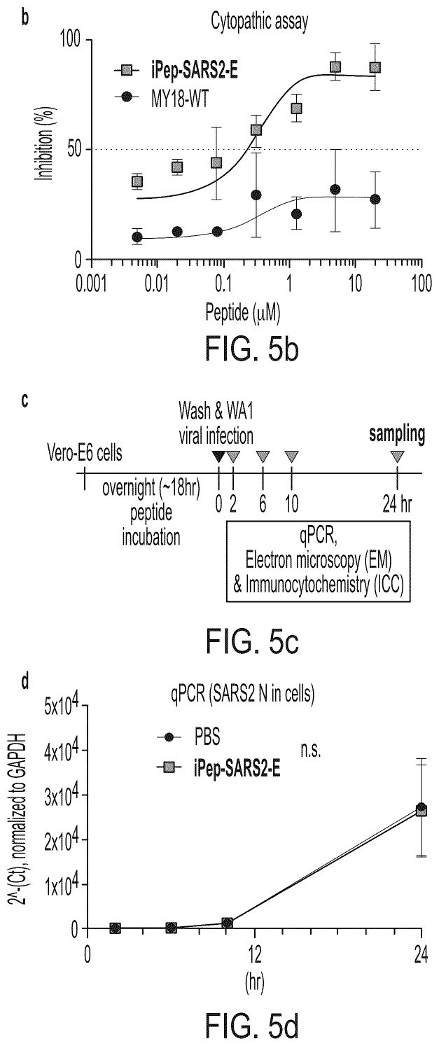

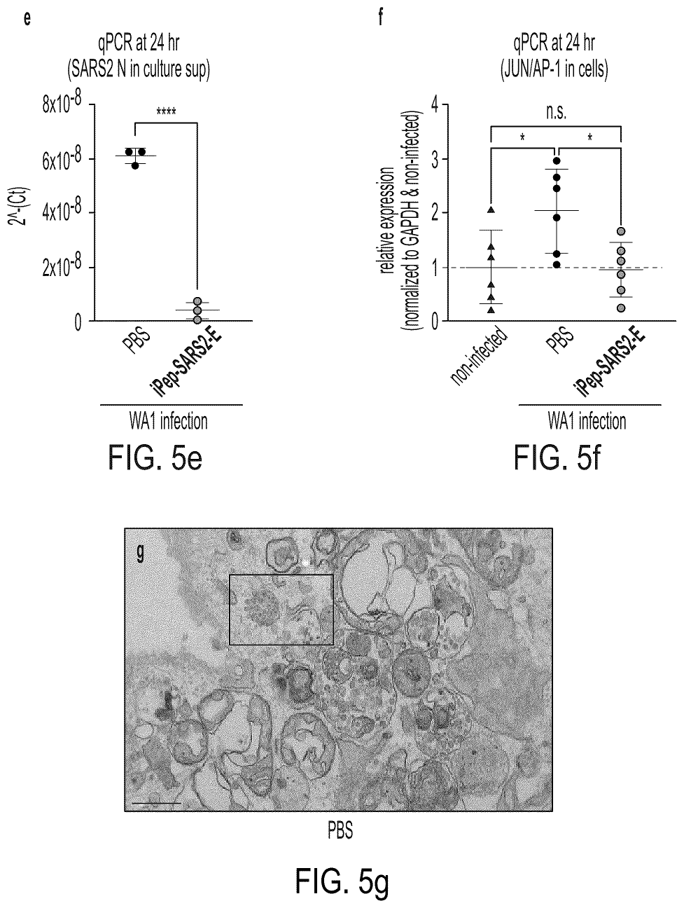

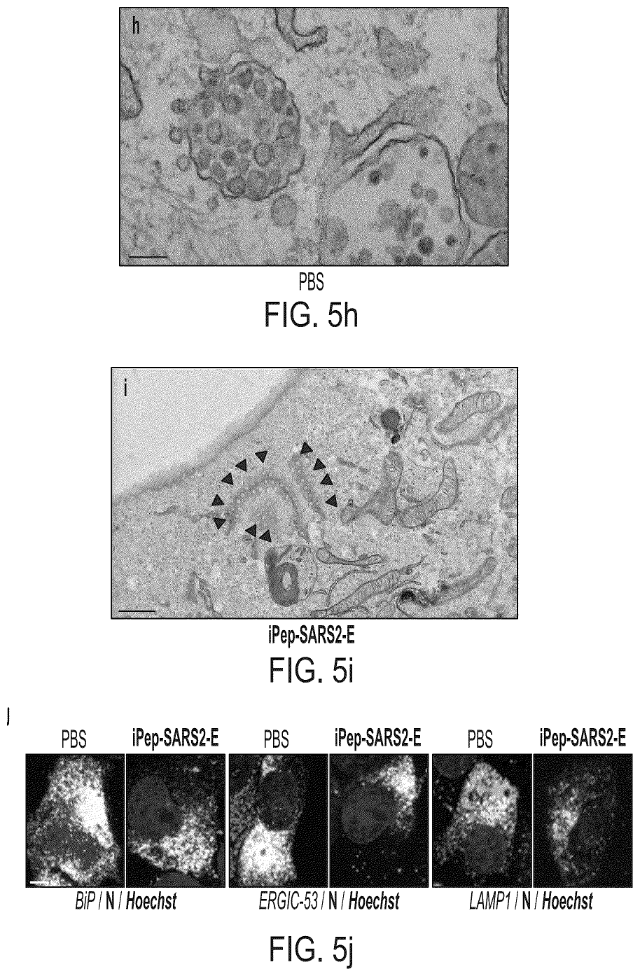

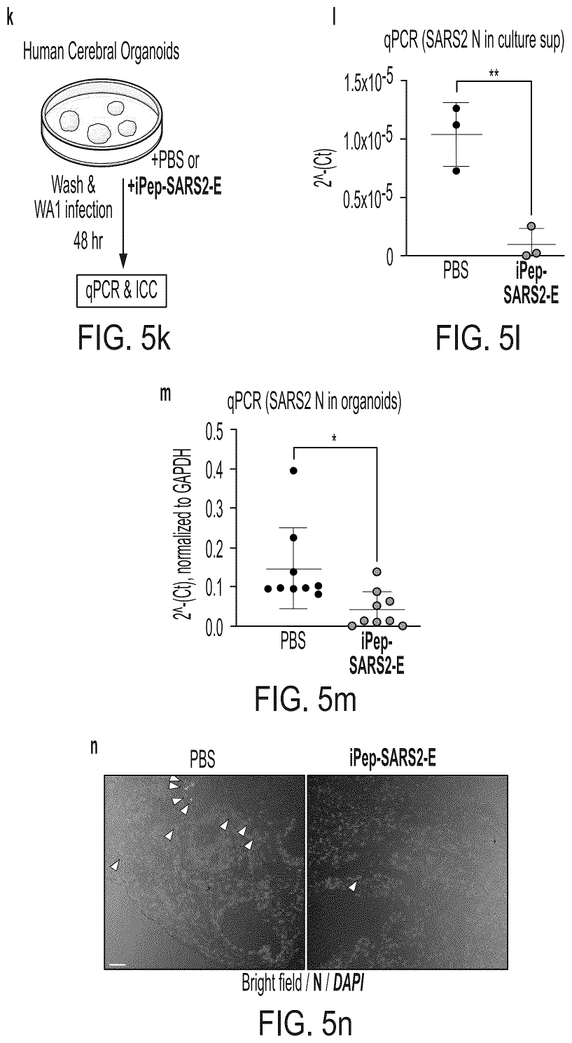

To examine the effect of iPep-SARS2-E (TAT-MY18-2ED) on SARS-CoV-2 virus, as a proof-of-concept experiment in vitro, we conducted cytopathic assay using a mammalian cell line, Vero-E6, and live SARS-CoV-2 virus (WA1 strain, FIG. 5a). As a negative control, we used MY18 wild-type peptide (non-TAT version, MY18-WT) in the assay because MY18-WT does not have any effect on 2E in the pH imaging (FIG. 1e). The cytopathic assay result demonstrates that iPep-SARS2-E significantly inhibits viral toxicity in vitro (FIG. 5b: IC₅₀ of iPep-SARS2-E, ˜200 nM). Following the results, next we designed and conducted time-course experiments to elucidate the mechanism underlying the inhibitory effect of iPep-SARS2-E on the viral function (FIG. 5c). The qPCR result demonstrates that there is no difference in SARS-CoV-2 viral gene expression, suggesting no effect of iPep-SARS2-E on the viral entry (FIG. 5d). On the other hand, there is a significant difference in SARS-CoV-2 viral gene detection of the culture supernatant samples harvested at 24 hours post-infection between PBS-treated control and iPep-SARS2-E-treated cells (FIG. 5e), demonstrating a significant reduction of viral release from iPep-SARS2-E-treated cells. Importantly, we found that iPep-SARS2-E could significantly restore the expression of JUN/AP-1 (FIG. 5f), which has been used as a reporter of SASR2-E cellular toxicity at this study to optimize MY18 peptide series (FIG. 2). Electron microscopy reveals that viral particles can be observed in large vacuoles of PBS-treated cells (FIG. 5g,h), which could be deacidified and disrupted lysosomes, according to the previous study²⁶. However, no vacuoles containing multiple viral particles were found in iPep-SARS2-E-treated cells while small particles were found in the endoplasmic reticulum and nuclear envelope (FIG. 5i), suggesting that the particles might be virions while it is not clear whether the virions are mature in iPep-SARS2-E-treated cells. Immunocytochemistry confirms that the nucleocapsid protein is co-localized to BiP/GRP78, a marker of endoplasmic reticulum (ER) and also ERGIC-53, a marker of ER-Golgi inter-compartment (ERGIC) in iPep-SARS2-E-treated cells while the nucleocapsid proteins in PBS-treated cells were highly and broadly expressed and also co-localized to LAMP1, a lysosomal marker (FIG. 5j). The results suggest that iPep-SARS2-E inhibits viral egress.

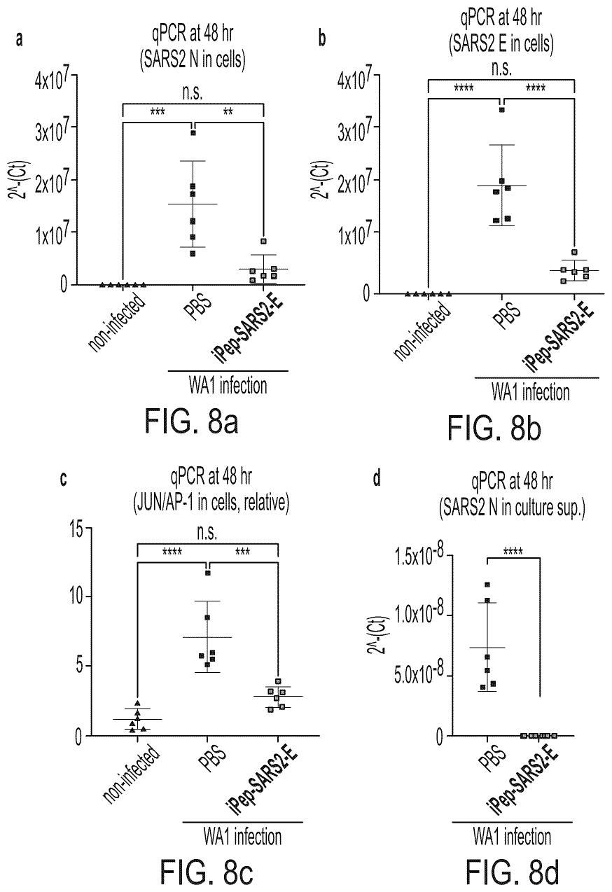

Following the results, we conducted in vitro experiments using iPep-SARS2-E at later time-point to examine the effect further. The qPCR result shows that there is a significant decrease in SARS-CoV-2 viral gene expressions (nucleocapsid and E) in iPep-SARS2-E-treated Vero-E6 cells at 48 hours post-infection compared to the PBS-treated control cells (FIG. 8a and FIG. 8b). In addition, iPep-SARS2-E could significantly restore the expression of JUN/AP-1 (FIG. 8c). Importantly, there is a significant reduction in SARS-CoV-2 viral gene detection of the culture supernatant harvested at 48 hours post-infection in iPep-SARS2-E-treated cells compared to the PBS-treated control cells (FIG. 8d), demonstrating that iPep-SARS2-E suppresses viral release. Immunocytochemistry demonstrates that the majority of infected Vero-E6 cells became round and apoptotic in PBS-treated group while iPep-SARS2-E-treated cells had moderate expression of viral nucleocapsids with normal cellular morphology, which is consistent with the cytopathic assay result (FIG. 5b).

To validate the inhibitory effect of iPep-SARS2-E peptide further, we conducted in vitro experiments using human cerebral organoids using WA1 virus (FIG. 5k). We found that there is a significant difference in SARS-CoV-2 viral detection of the culture supernatant at post-infection between PBS- and iPep-SARS2-E-treated organoids (FIG. 5l), suggesting that viral egress is blocked by iPep-SARS2-E. Also, SARS-CoV-2 viral gene expression was significantly reduced in iPep-SARS2-E-treated organoids at 48 hours post-infection compared to the PBS-treated control organoids (FIG. 5m). Immunocytochemistry allows us to observe higher expression of SARS-CoV-2 nucleocapsid compared to iPep-SARS2-E-treated organoid sections (FIG. 5n). The results using the organoids are consistent with the results using Vero-E6 cells at 48 hours post-infection (FIG. 8a and FIG. 8d). Our results in vitro reveal that iPep-SARS2-E can be useful to affect E and the viral egress as a new strategy to prevent SARS-CoV-2 propagation in not only monolayer cells but also 3D organoid model.

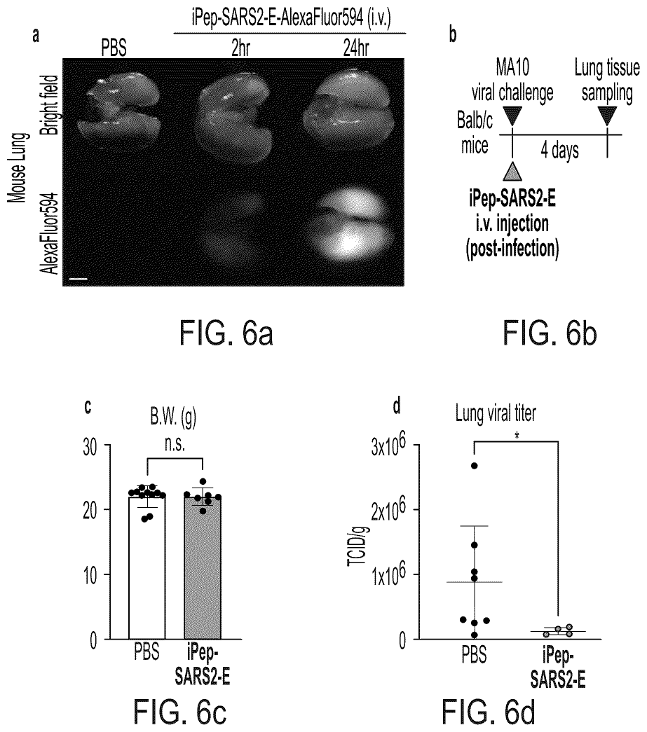

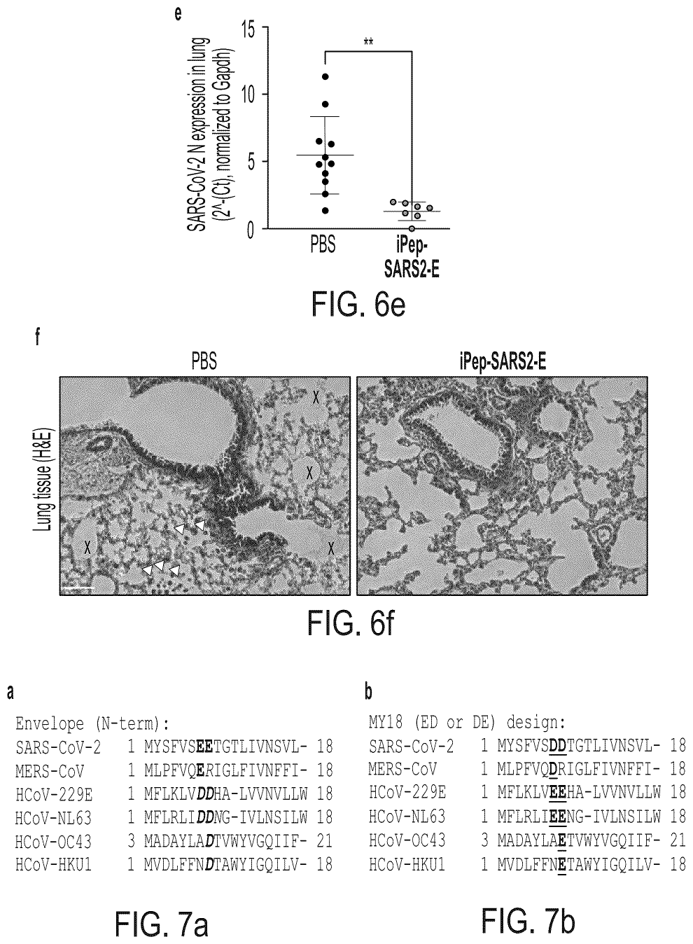

Next, to validate the inhibitory effect of iPep-SARS2-E further, we conducted a preclinical experiment in vivo using iPep-SARS2-E intravenous (i.v.) injection to Balb/c mice infected with MA10 virus, a mouse-adapted strain of SARS-CoV-2 virus²⁷. First, we conducted i.v. injection of the C-terminal version of iPep-SARS2-E fluorescent peptide (TAT-MY18-2ED-Alexa Fluor 594) to mice and confirmed that the peptide can permeate and is detectable in mouse lung tissues 2 hours after administration (FIG. 6a). Following the result, we next injected iPep-SARS2-E at post-infection. Four days after MA10 viral infection, the mice were sacrificed and their lungs were harvested for viral titer, qPCR and histology (FIG. 6b). The results show that there is no difference in body weight between the groups but a significant reduction of the viral propagation in iPep-SARS2-E-treated mouse lungs compared to the control (FIG. 6c, FIG. 6d). qPCR also confirms the inhibitory effect of iPep-SARS2-E peptide on the viral propagation in vivo (FIG. 6e). Lung histology reveal that no immune infiltration and alveolar damage are observed in iPep-SARS2-E-treated mouse lungs while minimal interstitial infiltrates with patchy lymphoid aggregates and protein accumulation were observed in the non-treated control group (FIG. 6f).

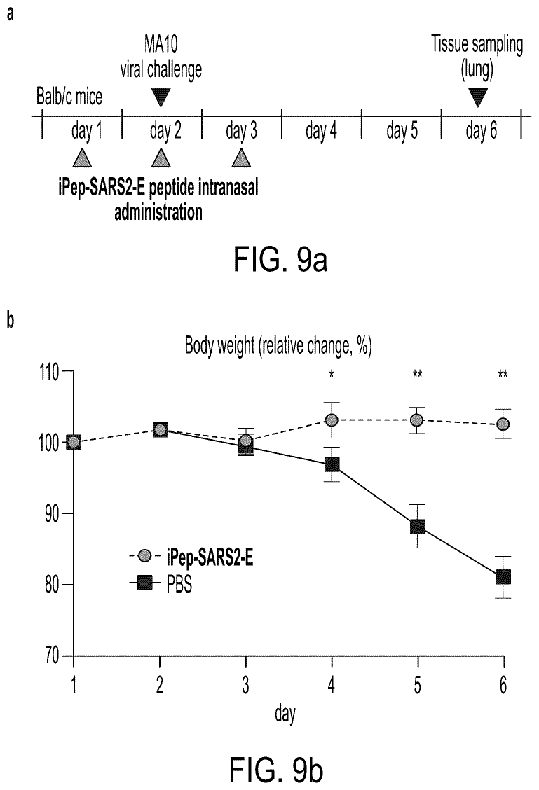

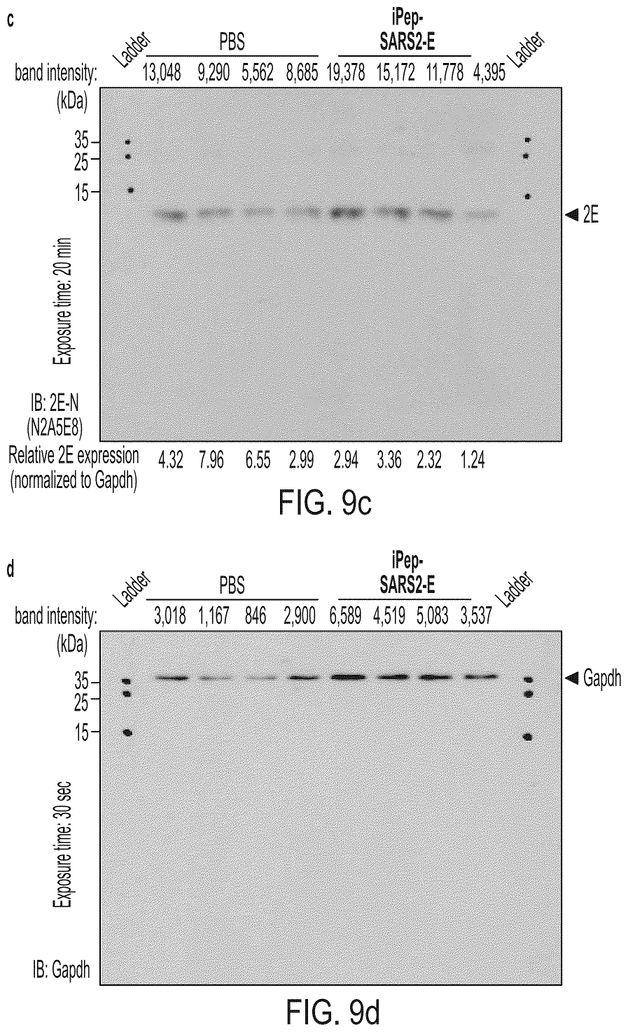

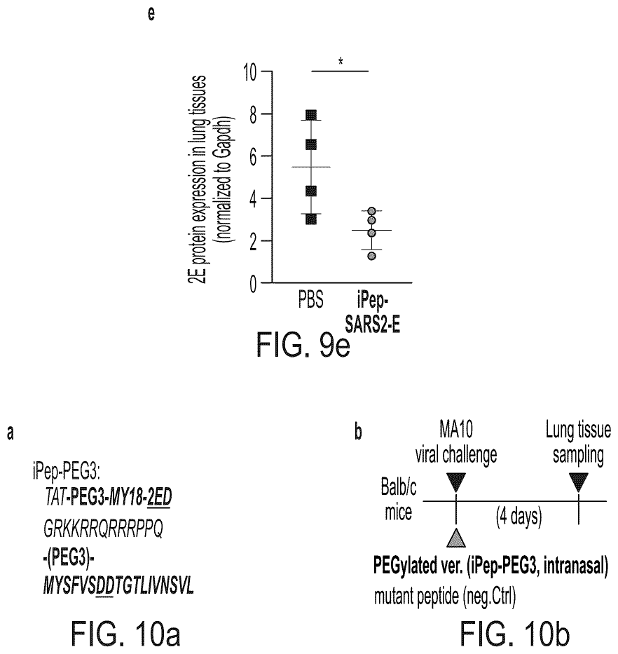

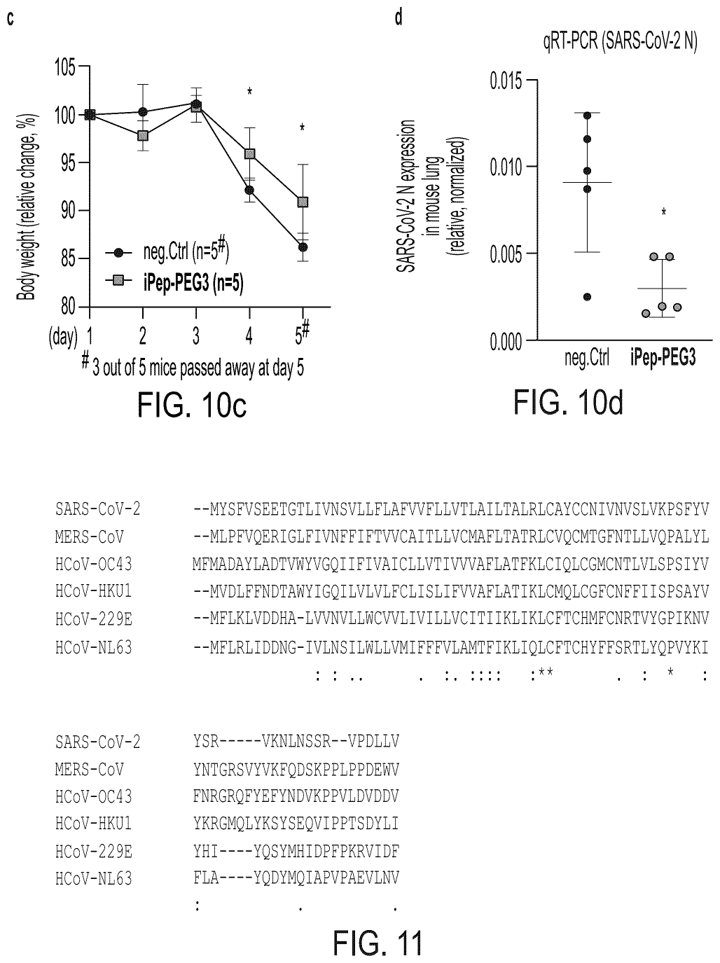

Following the result using i.v. injection, to examine whether iPep-SARS2-E could be applied for prevention of SARS-CoV-2 infection, we conducted another experimental series in vivo using iPep-SARS2-E intranasal administration to Balb/c mice infected with MA10 virus. First, we administrated the C-terminal version of iPep-SARS2-E fluorescent peptide to mice intranasally and confirmed that the peptide can permeate and is detectable in mouse nasal tissues 2 hours after administration. Next, we conducted a safety study in vivo using intranasal administration to Balb/c mice to examine the effect of iPep-SARS2-E on body weight and inflammation markers, comparing to non-treated and PBS-administrated groups. We did not find that there are differences in body weight, Cxc112 and C5a among iPep-SARS2-E, PBS, and non-treated groups. Following the results using intranasal administration, we applied iPep-SARS2-E intranasal administration to Balb/c mice infected with MA10 virus (FIG. 9a). We found that iPep-SARS2-E significantly prevents the body weight loss and suppresses 2E protein expression in infected mouse lungs in vivo (FIGS. 9b-9e). These results using in vivo experiments demonstrate that SARS2-E inhibition can be a novel strategy to prevent SARS-CoV-2 virus toxicity and propagation in vivo.

Our experiments to study the PEGylated peptide was conducted as follows. Balb/c mice (8-12 weeks old males, Charles River) were either mock (PBS) or infected intranasally with 5×10⁴ PFU of SARS-CoV-2 (MA10) in a final volume of 50 μl (a single dose) following isoflurane sedation. After viral infection, mice were monitored daily for body weight, temperature and foods. Mice showing >20% loss of their initial body weight were defined as reaching experimental end-point and humanely euthanized. The peptides were provided intravenously (i.v., 2 mM, 150 μl in PBS, pH7.0 adjusted with NaOH, a single dose) under isoflurane sedation, following a previous peptide-related study (de Vries, R. D. et al. Intranasal fusion inhibitory lipopeptide prevents direct-contact SARS-CoV-2 transmission in ferrets. Science 371, 1379-1382, (2021)). In case of the PEGylated and dead-mutant peptides, a single dose of intranasal administration was used together with the infection under isoflurane sedation (2.5 mM, 50 μl in PBS, pH7.0 adjusted with NaOH). The lung tissue samples were collected at the end-point (4 days post-infection) for RNA preparation, lung histology and/or lung viral titer using standard methods (de Vries et al. 2021) as well as our optimized method of SARS2-E protein blotting described hereinafter.

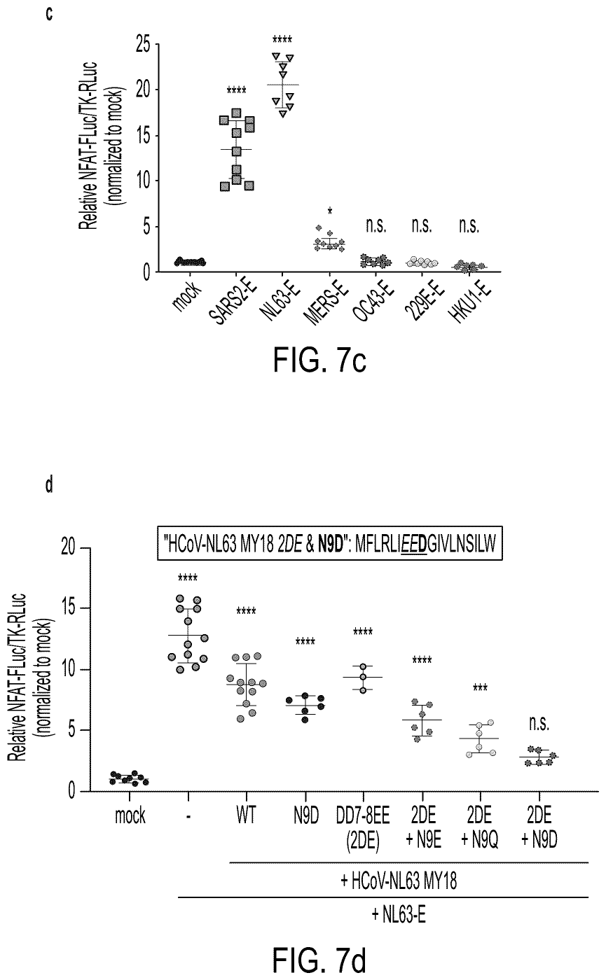

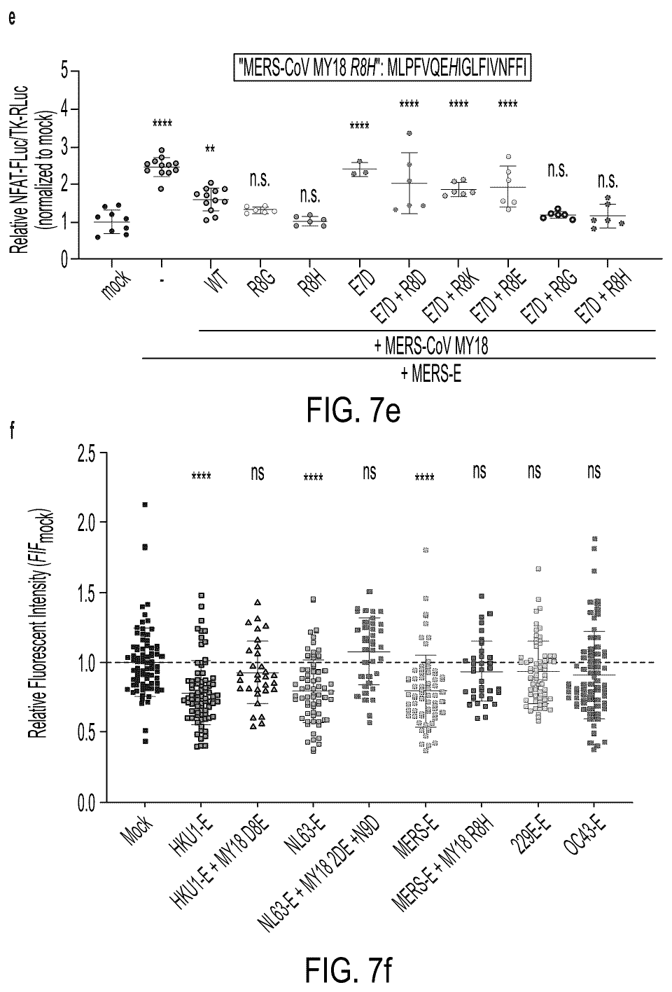

Next, we hypothesized that the peptide design and strategy could be applicable and customizable to the other human coronaviruses such as MERS-CoV because coronavirus E proteins are highly conserved (FIG. 7a and FIG. 11). Therefore, following iPep-SARS2-E development, we designed inhibitory peptide constructs against E proteins from each of the other human coronaviruses: MERS-CoV, HCoV-NL63, -OC43, -HKU1 and -229E (FIG. 7b). First, we found that the overexpression of MERS-CoV and HCoV-NL63 E proteins significantly increased NFAT/AP-1 luciferase reporter activity in mammalian cells while the E proteins of HCoV-OC43, -HKU1 and -229E do not have an effect on NFAT/AP-1 pathway (FIG. 7c). Following these results, we tested MER-CoVS and HCoV-NL63 MY18 WT peptide constructs using the NFAT/AP-1 luciferase reporter assay. The reporter assay results demonstrate that MERS-CoV and HCoV-NL63 MY18 WT could significantly reduce the effect of each E on NFAT/AP-1 reporter while substitution of Glu/E to Asp/D or Asp/D to Glu/E does not improve the MY18 constructs for MERS-CoV and HCoV-NL63, respectively (FIG. 7d, FIG. 7e). To improve each MY18 further, we conducted mutagenesis of MERS-CoV MY18 and HCoV-NL63 MY18 constructs. We found that HCoV-NL63 MY18 2DE & N9D and MERS-CoV MY18 R8H are effective to inhibit the cellular toxicity of HCoV-NL63 and MERS E proteins in mammalian cells, respectively (FIG. 7d, FIG. 7e). Next, using DND-189 pH imaging, we found that the overexpression of MERS-CoV, HCoV-NL63 and -HKU1 E proteins significantly reduced DND-189 fluorescence in mammalian cells while the Envelopes of HCoV-OC43 and -229E do not have a significant effect on lysosomal proton homeostasis (FIG. 7f). Following these results, we tested each MY18 construct and found that MERS-CoV MY18 R8H, HCoV-NL63 MY18 2DE & N9D, HCoV-HKU1 MY18 D8E peptide constructs could significantly rescue the phenotypes in proton homeostasis caused by MERS-CoV, HCoV-NL63 and HCoV-HKU1 E proteins in mammalian cells, respectively (FIG. 7f). The results of this experiment reveal that the MY18 peptides can be applicable for not only SARS-CoV-2 but also some of the other human coronaviruses such as MERS-CoV, CoV-NL63 and HCoV-HKU1, demonstrating that E can be a potential therapeutic target for human coronaviruses.

### DISCUSSION

We found that iPep-SARS2-E could rescue the molecular and cellular phenotypes in mammalian cells transfected with 2E. iPep-SARS2-E significantly reduces 2E-YFP protein expression in mammalian cells while having no effect on YFP and GAPDH protein (FIG. 3a-3d). Altered lysosomal pH due to 2E may result in a protective effect that enables for viral proteins such as Spike to be sequestered from proteolysis and promote viral assembly and release as reported in the other viruses^{19,26}. Therefore, 2E dysfunction with iPep-SARS2-E might result in 2E proteolysis conducted by normal lysosomal activity in the cells (FIG. 3f). Control safety experiments in vitro and in vivo demonstrate that there is no obvious toxicity of iPep-SARS2-E in vitro and in vivo. iPep-SARS2-E was shown to be efficacious for SARS-CoV-2 virus both in vitro and in vivo (FIGS. 5a-5n and FIGS. 6a-6e). The cytopathic assay result demonstrates that, although no cell-penetrating motif is added, MY18-WT, which was used as a negative control, also had a moderate inhibitory effect on the virus (FIG. 5b), suggesting that the non-TAT version might be uptaken by endocytosis in the cells spontaneously. Importantly, the in vitro preclinical study reveals that iPep-SARS2-E inhibits the viral egress since the viral transcript detection was significantly lower in iPep-SARS2-E-treated cell culture supernatant than PBS control (FIG. 5e). On the other hand, we found that there is no difference in SARS-CoV-2 nucleocapsid expression (FIG. 5d), suggesting no effect of iPep-SARS2-E on the viral entry. Because of the unique effect of iPep-SARS2-E, synergetic effect using iPep-SARS2-E with other therapeutic candidates such as protease and polymerase inhibitors might be promising. We further demonstrate that MY18 peptide design could be customizable for each human coronavirus E (FIGS. 7a-7f). Therefore, our approach using MY18 peptide series may be applied for future variants of human coronaviruses including SARS-CoV-2 new variants although further optimization of MY18 peptides is required.

In this study, we established and applied two screening platforms using lysosomal pH fluorescent imaging and the NFAT/AP-1 luminescence reporter system to identify novel therapeutic candidates against human coronavirus E. Our approach using synthetic peptides can be customizable and applicable for not only SARS-CoV-2 but also the other viruses, such as MERS-CoV, HCoV-NL63 and HCoV-HKU1, demonstrating that E is a potential therapeutic target for human coronaviruses.

### Methods

### Experimental Model and Subject Details

All rodent experimental procedures were carried out in accordance with regulations and established guidelines and were reviewed and approved by the Institutional Animal Care and Use Committee and BSL-3 Facility Committee at Columbia University (#AC-AABP2571).

### Cell Culture

Human embryonic kidney (HEK) 293S cells (ATCC, Cat #CRL-3022) were cultured in Dulbecco's Modified Eagle Media Nutrient Mixture F-12 (DMEM/F-12, Thermo-Fisher Gibco #11320033) and Human embryonic kidney (HEK) 293T cells (ATCC, Cat #CRL-3216), NIH 3T3 cells (ATCC, Cat #CRL-1658), and Vero-E6 cells (Catalog #CRL-1586) were cultured in and Dulbecco's Modified Eagle Media (DMEM, Thermo-Fisher/Gibco #10313021). Both media were supplemented with GlutaMax-I and penicillin/streptomycin (PS) and 10% fetal bovine serum (FBS, not heat-inactivated, HyClone, #SH30071.03, Thermo-Fisher) under normoxia (20% O₂, 5% CO₂, at 37° C.). The cell lines were passaged using trypsin-EDTA (0.25%, Thermo-Fisher, #25200-056) every 2-3 day. Human brain organoids were prepared using an established method with our normal induced pluripotent stem cell lines²⁸ and STEMdiff Cerebral Organoid Kit (STEMCELL Technologies, #8570).

### Molecular Biology Constructs

Plasmid DNA constructs were generated using standard methods with restriction enzymes (New England BioLabs), DNA ligase (MightyMix, TaKaRa Bio/Clontech) and polymerase chain reaction (PCR) with Phusion polymerase (Thermo-Fisher). Construct inserts for these experiments were synthesized (Integrated DNA Technologies, IDT) and subcloned into pcDNA3 vector (Life Technologies). Mock transfections were performed by using pcDNA3 empty vector.

### Imaging Experiments and pH Measurements

For the imaging experiments in FIGS. 1a-1e and 7a-7f, NIH 3T3 cells were plated at a cell density of 2.5×10⁵ cells/ml. The following day, the cells were transfected using Lipofectamine 3000 reagents (Thermo-Fisher, #L3000001). 4 μg of plasmid were mixed into 125 μL of serum-free OptiMEM with 5 μL of P3000 reagent. This was then added to another 125 μL of serum-free OptiMEM containing 7.5 μL of Lipofectamine 3000. Plasmid/P3000-lipofectamine complex was incubated for 15 minutes at room temperature, and then added to the plate. The medium was replaced 20-24 h after transfection, and 1 μM of Lysosensor Green DND-189 (Thermo-Fisher, L7535) was added. The cells were incubated for 30 minutes in a 37° C., 5% CO₂ incubator. The medium was replaced one final time prior to imaging. The live cell imaging was conducted on a customized/automated fluorescence microscope (Ti—U, Nikon) using an environmental chamber (TOKAI) and the culture medium to maintain normal cell culture conditions (37° C., 5% CO₂, 20% O₂). Transfection efficiency was estimated by counting cells that showed mKate2 red fluorescence and was typically between 25 and 35%. Fluorescence quantification and analysis was performed with ImageJ software and Prism 7/8/9 (GraphPad). Representative images were also gathered on a Leica DMi8 confocal microscope. The following peptides (>95% purity) were synthesized by Thermo-Fisher/Pierce:

MY18:
MYSFVSEETGTLIVNSVL
6-Arg (Arg)-MY18:
RRRRRR-MYSFVSEETGTLIVNSVL
TAT-MY18:
GRKKRRQRRRPPQ-MYSFVSEETGTLIVNSVL
Penetratin (Pen)-MY18:
RQIKIWFQNRRMKWKK-MYSFVSEETGTLIVNSVL

### Global Quantitative Proteomics by Mass Spectrometry

For global quantitative proteomics of HEK 293S transfected using Lipofectamine 2000 (Invitrogen 11668027) with pcDNA3-SARS-CoV-2 Envelope (WT)-mKate2, tandem mass tag (TMT)-based quantitative proteomics was used. In brief, frozen cells were lysed by bead-beating in 9M urea and 200 mM EPPS (pH 8.5), supplemented with protease and phosphatase inhibitors. Samples were reduced with 5 mM TCEP and alkylated with 10 mM iodoacetamide (IAA) that was quenched with 10 mM DTT. A total of 100 μg of protein was chloroform-methanol precipitated. Protein was reconstituted in 200 mM EPPS (pH 8.5) and digested by Lys-C overnight and trypsin for 6 h, both at a 1:50 protease-to-peptide ratio. Digested peptides were quantified using a Nanodrop at 280 nm and 50 μg of peptide from each sample were labeled with 400 μg TMT reagent using 10-plex TMT kit²⁹. TMT labels were checked, 0.5 μg of each sample was pooled, desalted and analyzed by short SPS-MS3 method, and using normalization factor, samples were bulk mixed at 1:1 across all channels and 500 μg of the bulk mixed sample was used for total proteome analysis. Mixed TMT-labeled samples were vacuum centrifuged and desalted with C18 Sep-Pak (100 mg) solid-phase extraction column. The desalted sample was fractionated using BPRP chromatography. Peptides were subjected to a 50 min linear gradient from 5 to 42% acetonitrile in 10 mM ammonium bicarbonate pH 8 at a flow rate of 0.6 mL/min over Water X-bridge C18 column (3.5 μm particles, 4.6 mm ID and 250 mm in length). The peptide mixture was fractionated into a total of 96 fractions, which were consolidated into 28 fractions. Fractions were subsequently acidified with 1% formic acid, and vacuum centrifuged to near dryness and desalted via SDB-RP StageTip.

For total proteome analysis, 28 desalted fractions were dissolved in 10 μl of 3% acetonitrile/0.1% formic acid injected using SPS-MS3. The UltiMate 3000 UHPLC system (Thermo-Fisher) and EASY-Spray PepMap RSLC C18 50 cm×75 μm ID column (Thermo-Fisher) coupled with Orbitrap Fusion (Thermo-Fisher) were used to separate fractioned peptides with a 5-30% acetonitrile gradient in 0.1% formic acid over 45 min at a flow rate of 250 nL/min. After each gradient, the column was washed with 90% buffer B for 10 min and re-equilibrated with 98% buffer A (0.1% formic acid, 100% HPLC-grade water) for 40 min. The full MS spectra were acquired in the Orbitrap Fusion™ Tribrid™ Mass Spectrometer (Thermo-Fisher) at a resolution of 120,000. The 10 most intense MS1 ions were selected for MS2 analysis. The isolation width was set at 0.7 Da and isolated precursors were fragmented by CID at normalized collision energy (NCE) of 35% and analyzed in the ion trap using “turbo” scan speed. Following the acquisition of each MS2 spectrum, a synchronous precursor selection (SPS) MS3 scan was collected on the top 10 most intense ions in the MS2 spectrum. SPS-MS3 precursors were fragmented by higher energy collision-induced dissociation (HCD) at an NCE of 65% and analyzed using the Orbitrap.

Raw mass spectrometric data were analyzed using Proteome Discoverer 2.4 to perform database search and TMT reporter ions quantification. TMT tags on lysine residues and peptide N termini (+229.163 Da) and the carbamidomethylation of cysteine residues (+57.021 Da) was set as static modifications, while the oxidation of methionine residues (+15.995 Da), deamidation (+0.984) on asparagine and glutamine were set as a variable modification. Data were searched against a UniProt human with peptide-spectrum match (PSMs) and protein-level at 1% FDR. The signal-to-noise (S/N) measurements of each protein were normalized so that the sum of the signal for all proteins in each channel was equivalent to account for equal protein loading. Results obtained from PD2.4 were further analyzed using Perseus statistical package³⁰ is part of the MaxQuant distribution. Significantly changed protein abundance was determined by ANOVA with P<0.05 (permutation-based FDR correction). Pathway analysis was performed using ingenuity IPA (Qiagen).

### Luciferase Assay

HEK 293T cells were plated at 0.5×10⁵ cells/well in 24 well plates (Corning) coated with poly-ornithine (Sigma-Aldrich). The following day, the cells were transfected with DNAs encoding the envelope protein of interest, an NFAT Firefly Luc (NFAT-FLuc) reporter (using 4× NFAT site from human IL-2 gene), and pRL-TK-Renilla Luc reporter (TK-RLuc, transfection control reporter using HSV TK, herpes simplex virus thymidine kinase, promoter) using Lipofectamine 2000 reagents (Invitrogen/Life Technologies #11668027). The standard transfection ratio of Envelope protein: NFAT-FLuc:TK-RLuc was as follows: 0.3 μg:0.3 μg:0.03 μg. Peptides of interested were added to this mixture at an amount of 0.1 μg. The cells were then incubated overnight at 37° C. in a CO₂ incubator. The following day the cells were treated with 1 μM Phorbol 12-myristate 13-acetate (Sigma-Aldrich, P1585) for 8 hours at 37° C. in a CO₂ incubator. Luc activity levels were then assayed using Dual Luciferase assay kit and Veritas 96-well luminometer (Promega, E1910) following the manufacturer's instructions. Following the luciferase results, TAT-MY18-2ED peptide (>95% purity) was synthesized by Thermo-Fisher/Pierce Custom Peptide team:

TAT-MY18-2ED:
GRKKRRQRRRPPQ-MYSFVSDDTGTLIVNSVL
(M.W. = 3,662.2416)

### SARS-CoV-2 Envelope Western Blotting

Western blot experiments were conducted using standard method. In brief, HEK 293T cells were plated in a 6-well dish at a 1×10⁶ cells/well density. Cell samples receiving iPep-SARS2-E peptide (10 μM) were treated with the peptide for 24 hours prior to transfection and peptide was refreshed during transfection. For transfection, 4 μg of the plasmid were mixed into 125 μL of serum-free OptiMEM, and 5 μL of Lipofectamine 2000 added to another 125 μL of serum-free OptiMEM. The two pots were combined and incubated at room temperature for 15 minutes. The next day, cells were lysed in 10× cell lysis buffer (Cell Signaling Technology, #9803) with 1% protease inhibitor cocktail (Sigma-Aldrich/Millipore-Sigma). Samples were denatured using an SDS-urea solution and boiled at 95° C. for 5 min. SDS-polyacrylamide gel electrophoresis was performed using Tris-Glycine-based gels (Bio-Rad) containing 10% Acrylamide-Bis (Fisher Scientific) which was then transferred to polyvinylidene difluoride (PVDF) membranes. Primary antibodies to GFP (MBL #598, 1/8,000 dilution) and anti-GAPDH (rabbit recombinant monoclonal antibody, ab181602, 1:10,000 dilution, Abcam). Secondary antibody α-rabbit (Thermo-Fisher, #31430, 1/8,000 dilution). 5% skim milk in TBS-T was used for blocking of the PVDF membranes. Pierce ECL Western blotting substrate (Thermo-Fisher, #32209) was used for the chemiluminescent reaction.

### Monoclonal Antibody Production

Immunization of animals: The emulsion was prepared by mixing synthesized SARS2-E N-term peptide (Thermo-Fisher) in 50% DMSO in PBS and the adjuvant complete freund (BD, #263810) evenly to make final peptide concentration of 1 mg/mL. Eight-week-old WKY/NCr1 female rat purchased from Charles River Laboratories (Massachusetts, USA) was injected intramuscularly at the right and left tail base with 100 μl each of emulsion (total 200 μg peptide/rat) under our animal protocol (AC-AABC3508). Three times booster injections were done using the same method except at days 14, 17, and 20 where adjuvant incomplete Freund (BD, #963910) was used instead of complete Freund.

Lymphocyte harvest: As a partner cell, mouse myeloma cell line NS-1 (P3/NS1/1-Ag4.1, Sigma-Aldrich, #85011427-1VL) was used. NS-1 cells were maintained with NS-1 medium (DMEM with 20% FBS, 1× GlutaMax supplement and 1× Pen. Strep.; Gibco #10313-021, HyClone #SH30071.03, Gibco #35050-061 and Gibco #15140-122, respectively) at 37° C., 5% CO2. Two days after the last antigen injection, the rat was euthanized and the medial iliac lymph node was taken aseptically and placed into 1 ml of NS-1 medium and cut to small pieces using sterile surgical blades. Following gentle pipetting to separate lymphocyte cells from other tissues, the lymphocyte cells were strained using a 100 μm pore cell strainer (Falcon, #352360). After counting, lymphocytes were frozen in NS-1 medium with 10% DMSO and kept in liquid nitrogen for storage.

Cell fusion: Lymphocytes were initiated the day before fusion and cultured in NS-1 medium. Fifteen million lymphocytes and 30 million NS-1 cells were mixed and spun down at 500×g for 2 min. After washing with HBSS (Gibco, #14175095), the cell pellet was resuspended in the fusion medium (0.3M mannitol with 0.1 mM CaCl₂ and MgCl₂). The mixture was then put into the fusion chamber and fused using an electrofusion method (NAPA GENE, #ECFG21, for align; 30V for 20 sec. For fusion; 350V for 30 μsec 3 times with 0.5 sec interval). The mixture was then collected from the fusion chamber and spun down at 500×g for 2 minutes. The fusion pellet was then resuspended to 1^{st} culture medium (fresh NS-1 medium mixed with equal volume NS-1 cultured conditioned medium with 1× Hymax Hybridoma Fusion & Cloning Supplement (Antibody Research Corporation, Missouri, USA, #113004), and plated on a 96 well plate. Hybridomas were selected by HAT sectioning culture for 2 weeks with NS-1 medium with 1× HAT media supplement (Sigma-Aldrich, #H0262) and 1× Hymax supplement, followed by HT maintenance culture with NS-1 medium with 1× HT media supplement (Sigma-Aldrich, #H0137) and 1× Hymax solution.

Screening: Primary screening was done by immunocytochemistry using 2E-YFP expressing plasmid transfected NIH 3T3 cells. The cells were plated on a Nunc Lab-Tek II chamber slide (Thermo-Fisher, #154534) at a density of 15,000 cells/well and transfected with plasmid using Lipofectamine 3000 (Invitrogen, #L3000001) following the manufacture's protocol. Twenty hours later, the cells were fixed using 4% paraformaldehyde in PBS. After 3 times wash with PBS, the cells were incubated with hybridoma culture supernatant with 0.5% NP-40 for 1 hour at room temperature. After 3 times wash with PBS, the cells were incubated with anti-rat IgG secondary antibody conjugated with Alexa Fluor 594 (1:2,000 dilution, Abcam, #ab150160) for 30 min at room temperature. Positive clones were expanded to 24 well plate. After reaching 80% confluency, western blot was done as a secondary screening. For western blot preparation, SARS2-E-YFP plasmid was transfected into HEK 293T cells in a 10 cm dish using Lipofectamine 2000 following manufacture's protocol. Twenty-four hours later, the cells were lysed using lysis buffer (Cell Signaling, #9803S) and spun down to collect the supernatant. The supernatant was mixed with same volume of 2× SDS sample buffer (8M urea, 40 mM Tris-Cl (pH6.8), 2% SDS, 10% 2-mercaptoethanol) and boiled at 95° C. for 5 min. Following the SDS page using 10% Bis-acrylamide gel, the protein was transferred to a PVDF membrane by electroblotting. Following 1 h blocking by 5% milk in TBS-T, the membrane was cut longitudinally into 0.5 cm wide strip. The strips were incubated with hybridoma culture supernatant for 1 h at room temperature. After 3 times washing with TBS-T, the strips were incubated with anti-rat IgG secondary antibody conjugated with HRP (Invitrogen, #31470) for 30 min at room temperature. After 3 times washing, development was done using ECL solution (Thermo-Fisher, #32209) following the manufacture's protocol. Anti-GFP antibody (MBL, #598) was used as a positive control. If positive, single cell cloning was done using a limiting dilution method and another round of immunocytochemistry and western blotting was completed to confirm the result. Following immunocytochemistry and western blotting confirmation for the single cloned hybridomas, positive clones were passaged with gradually reduced concentration of Hymax supplement (½, ¼, 1/10, and finally without Hymax) until the hybridomas can be maintained by NS-1 medium with 1× HT solution. Hybridoma isotyping was done using rat isotyping kit (Bio-Rad, #RMT1).

Antibody purification: 50 mL of hybridoma culture supernatant was collected followed by filtration using a 0.22 μm filter. The culture supernatant was mixed an equal volume of 20 mM NaPi solution (pH7.0), with additions of NaCl (150 mM, final conc.) and Tween-20 (0.02%, final conc.) as well. 0.25 mL of Protein-G agarose (Thermo-Fisher, #20399) was added and gently rocked for 1 hour at room temperature. Protein-G agarose was packed in a column using an open column method and washed with 20 mL of 20 mM NaPi solution (pH7.0). 100 mM Glycine-Cl solution (pH2.7) was used as elution buffer and eluted solution was immediately neutralized by 1/10 volume of 1M Tris-HCl solution (pH8.0). The neutralized antibody solution was concentrated, and buffer changed to 20 mM NaPi with 0.25M NaCl solution using an Amicon Ultra-4 filter (size of 3K, Millipore, #UFC800396). Antibody concentration was measured using a rat IgG ELISA kit (Abcam, #ab189578). Anti-His tag antibody (R&D, MAB050-100) was used to detect 6×His tagged 2E recombinant proteins purified from a standard bacteria expression system using pCold IV vector (TaKaRa Bio/Clontech), Ni Sepharose 6FF (Sigma-Aldrich, #17-5318-01) and Rosetta 2(DE3) pLysS competent cells (Novagen/Millipore-Sigma, 71400-3) using our established method³¹.

### Peptide Permeability Assay

The peptides (>95% purity) were synthesized by Thermo-Fisher/Pierce Custom Peptide team. Peptides were resolved in water and stored at a 2.5 mM stock concentration. For permeability assay NIH 3T3 and Vero-E6 cells were plated on 35 mm dishes at a density of 1×10⁵ cells/mL. ON kinetics: peptides were added to cell culture medium at a concentration of 10 μM and incubated under normoxia conditions until timepoints for imaging. OFF kinetics: peptides were added to the cell medium at a concentration of 10 μM and incubated under normoxia conditions. After 24 hours the medium was replaced (no new peptide added). Cells were then imaged at determined time points. For all imaging, cell media was replaced with Tyrode's solution (140 mM NaCl, 5.4 mM KCl, 1 mM MgCl₂, 10 mM glucose, 1.8 mM CaCl₂) and 10 mM HEPES, pH buffered to 7.4 with NaOH).

Alexa594-conjugated peptide:
AlexaFluo594-TAT-MY18-2ED-:
AlexaFluo594-[C]G-GRKKRRQRRRPPQ-
MYSFVSDDTGTLIVNSVL
TAT-MY18-2ED-AlexaFluo594:
GRKKRRQRRRPPQ-MYSFVSDDTGTLIVNSVL-
L[C]AlexaFluo594

### Stability Assay of Peptides

Peptide was synthesized by Thermo-Scientific/Pierce and resolved in water at a 2.5 mM stock concentration and aliquoted for storage at −20° C. Peptides were thawed on ice and diluted to 40 μg/mL using PBS. Samples were incubated at 37° C. Baseline levels were measured using freshly thawed peptide diluted by 37° C. pre-warmed PBS. Samples were transferred to an EIA/RIA plate (Corning, #3591) at 50 μL/well and incubated overnight for coating at 4° C. The next day, the plate was washed using 100 μL/well of PBS-T 3 times. The plate was blocked for 1 hour at room temperature using 3% bovine serum albumin (BSA, Sigma-Aldrich) in PBS-T with 250 rpm shaking. Following this, the plate was washed 3 times using PBS-T. The primary antibody solution was then added at 50 μL/well (N2A5E8, 0.45 μg/mL) for 1 h at room temperature with 250 rpm shaking followed by 3 times wash with PBS-T. The anti-rat IgG secondary antibody HRP conjugated solution (1:10,000, Invitrogen, #31460) was then added for 1 h at room temperature with 250 rpm shaking followed by 3 times wash with PBS-T. The development step was done using TMB solution (Thermo-Fisher, #34021) following the manufacture's manual. OD600 value was detected using a SpectraMax iD3 Plate Reader (Molecular Devices, San Jose, USA). Statistical analysis was done using GraphPad Prism software. This procedure was done in biological triplicate.

### Apoptosis/Necrosis Assay Using Peptides

Jurkat cells (ATCC, #TIB-152, cloneE6-1) were cultured and treated for 48 hours with 10 μM of iPep-SARS2-E peptide (TAT-MY18-2ED) or for 3 hours with 10 μM (S)-(+)-camptothecin (positive control group, Sigma-Aldrich, #C9911). After treatment, the cells were collected for an Annexin-V assay (Thermo-Fisher/Invitrogen, #V13241) following the manufacturer's manual. The data was collected by a ZE5 Cell Analyzer (Bio-Rad Laboratories, Inc.) and analyzed using FlowJo software (BD Biosciences). This procedure was done in biological triplicate.

### Viral Infection In Vitro and Sample Processing

Cytopathic assay using WA10 virus (MOI, 0.10) in Vero-E6 cells was conducted as done in the previous study using standard methods⁵. Immunocytochemistry was conducted using a standard method using fixation solution containing 4% paraformaldehyde (Electron Microscopy Sciences) and 2% sucrose (Sigma-Aldrich) in PBS, blocking/permeability solution (2% BSA and 0.25% NP-40, Sigma-Aldrich, in PBS) and antibodies to ERGIC/p58 (Sigma-Aldrich, E1031), LAMP1 (Abcam, ab24170), BiP/GPR78 (Abcam, ab21685) and SARS-CoV-2 nucleocapsid (Thermo-Fisher, PIMA17404). Goat anti-mouse IgG Alexa Fluor 594 antibody (Abcam, ab150116) and Goat anti-rabbit IgG Alexa Fluor 488 antibody (Abcam, ab150077). For electron microscopy, infected Vero-E6 cells were fixed using 2% paraformaldehyde, 2% glutaraldehyde (Electron Microscopy Sciences) and 2 mM CaCl₂ (Sigma-Aldrich) in 100 mM cacodylate buffer (pH 7.4, Electron Microscopy Sciences).

### Quantitative RT-PCR

RNA samples of Vero-E6 cells and mouse lung tissues were prepared using TRIzol Plus RNA Purification kit and PureLink DNase set (Thermo-Fisher) while RNeasy Mini kit and RNase-Free DNase set (Qiagen) was used for HEK 293 cells. cDNA was synthesized using the SuperScript III First-Strand Synthesis System for RT-PCR (Thermo-Fisher). FAST or Power SYBR™ Green PCR Master Mix and StepOnePlus real time PCR systems (Thermo-Fisher) with StepOne software (version 2.3, Life Technologies) were used for qPCR using the below primer sets.

SARS-CoV-2 qPCR N forward primer:
CTCTTGTAGATCTGTTCTCTAAACGAAC
SARS-CoV-2 qPCR N reverse primer:
GGTCCACCAAACGTAATGCG
SARS-CoV-2 qPCR E forward primer:
CTCATTCGTTTCGGAAGAGACAG
SARS-CoV-2 qPCR E reverse primer:
AGACCAGAAGATCAGGAACTCTAG
Mouse Gapdh qPCR forward primer:
CTTCACCACCATGGAGAAGG
Mouse Gapdh qPCR reverse primer:
TGAAGTCGCAGGAGACAACC
Monkey qPCR GAPDH (for Vero-E6) forward primer:
GAAGGTGAAGGTCGGAGTCAAC
Monkey qPCR GAPDH (for Vero-E6) reverse primer:
TCGTTGTCATACCAGGAAATGAGC
Human NFATC4 qPCR forward primer:
CTTCTCCGATGCCTCTGACG
Human NFATC4 qPCR reverse primer:
CGGGGCTTGGACCATACAG
Human JUN/AP-1 qPCR forward primer:
ACTCGGACCTTCTCACGTC
Human JUN/AP-1 qPCR reverse primer:
GGTCGGTGTAGTGGTGATGT
Human GAPDH qPCR forward primer:
CTCACCGGATGCACCAATGTT
Human GAPDH qPCR reverse primer:
CGCGTTGCTCACAATGTTCAT

### Viral Infection In Vivo and Sample Processing

Balb/c mice (8-11 weeks old males, Charles River) were either mock (PBS) or infected intranasally with 5×10⁴ PFU of SARS-CoV-2 (MA10) in a final volume of 50 μl (a single dose) following isoflurane sedation. After viral infection, mice were monitored daily for body weight, temperature and foods. Mice showing >20% loss of their initial body weight were defined as reaching experimental end-point and humanely euthanized. The peptides were provided intranasally with 2.7 mg/kg (total three doses) under isoflurane sedation or intravenously (i.v., 2 mM, 150 μl in PBS, pH7.0 adjusted with NaOH, a single dose), following a previous peptide-related study⁹. The lung tissue samples were collected at the end-point (4 days post-infection) for RNA preparation, lung histology and/or lung viral titer using standard methods⁹ as well as our optimized method of SARS2-E protein blotting described as the next section.

### Lung Tissue Western Blotting

To inactivate the virus, MA10-infected mouse lung was incubated in 0.5% SDS in PBS for 1 h at room temperature. After mashed by plastic masher, the sample mixture was spun down to collect the supernatant. The supernatant was mixed with same volume of 2× SDS sample buffer (8M urea, 40 mM Tris-Cl (pH6.8), 2% SDS, 10% 2-mercaptoethanol) and boiled at 95° C. for 5 min. Following the SDS page using 20% Bis-acrylamide gel, the protein was transferred to a PVDF membrane by electroblotting. Following 1 h blocking by 5% skim milk in TBS-T, the membrane was incubated with the primary antibody solution (N2A5E8, 0.2 μg/mL in TBS-T) for overnight at 4° C. After 3 times washing with TBS-T, the membrane was incubated with anti-rat IgG secondary antibody conjugated with HRP (Invitrogen, #31470) for 1 h at room temperature. After 3 times washing, development was done using ECL solution (Thermo-Fisher, #32209) following the manufacture's protocol.

### Fluorescent Stereoscopic Imaging of Mouse Tissues

iPep-SARS2-E peptide conjugated with Alexa594 at the C-terminus end (TAT-MY18-2ED-A594, 10 μM, 20 μL) or PBS was provided intranasally under the Isoflurane anesthesia. Two hours later, the mice were euthanized using CO₂. The taken skull was cut with sagittal section in the middle, followed by taken out the nasal septum. After the 3 times wash in the PBS, imaging for lateral side of nasal cavity was conducted on a fluorescent stereoscope (Leica, M165 FC). iPep-AF594 iv organs imaging. Regarding i.v. injection method, iPep-SARS2-E group mice were injected with TAT-MY18-2ED-A594 peptide (300 μM, 50 μL) via tail vein before 24 h or 2 h of sacrifice. The control mouse was injected with PBS before 2 h of sacrifice. After sacrifice using CO₂ and cervical dislocation, whole body perfusion with 15 mL of PBS was conducted to wash out the peptides in their bloods. Harvested tissues, such as lungs, were briefly washed by PBS and conducted imaging on the fluorescent stereoscope.

### Cytokine/Inflammation Array

Cytokine inflammation panel was done in accordance with the manufacturer's protocol for the mouse cytokine array kit panel A (R&D Systems, Cat #ARY006). Serum samples used were collected from blood heat-inactivated at 65° C. for 30 min (to inactivate any possible viruses) and spun down at 15,000 rpm for 10 minutes. 50p L of blood serum samples were used for the assay. Cxc112, C5a, MCSF and CD54 were detected in the array using the denatured blood samples.

### Statistics and Reproducibility

The statistics used for every figure have been indicated in the corresponding figure legends. The Student's t-test (paired and unpaired) was conducted with the t-test functions in Microsoft Excel software. The Student's t-test was two-tailed. The one-way ANOVA with Tukey's, Sidak's, Bonferroni's or Dunnett's post-hoc multiple comparison analysis was conducted with the GraphPad Prism 6/7/8/9 software. All the data meet the assumptions of the statistical tests. All the samples used in this study were biological repeats, not technical repeats. All experiments were conducted using at least two independent experimental materials/cohorts to reproduce similar results. No samples were excluded from the analysis in this study. All the graphs in the figures are mean±s.d.

### REFERENCE

1 Sanyaolu, A. et al. Global Pandemicity of COVID-19: Situation Report as of Jun. 9, 2020. Infect Dis (Auckl) 14, 1178633721991260, doi:10.1177/1178633721991260 (2021).
2 Zhu, N. et al. A Novel Coronavirus from Patients with Pneumonia in China, 2019. N Engl J Med 382, 727-733, doi:10.1056/NEJMoa2001017 (2020).
3 Zhou, P. et al. A pneumonia outbreak associated with a new coronavirus of probable bat origin. Nature 579, 270-273, doi:10.1038/s41586-020-2012-7 (2020).
4 Liu, L. et al. Striking antibody evasion manifested by the Omicron variant of SARS-CoV-2. Nature 602, 676-681, doi:10.1038/s41586-021-04388-0 (2022).
5 Wang, P. et al. Antibody resistance of SARS-CoV-2 variants B.1.351 and B.1.1.7. Nature 593, 130-135, doi:10.1038/s41586-021-03398-2 (2021).
6 Harvey, W. T. et al. SARS-CoV-2 variants, spike mutations and immune escape. Nat Rev Microbiol 19, 409-424, doi:10.1038/s41579-021-00573-0 (2021).
7 Andrews, N. et al. Covid-19 Vaccine Effectiveness against the Omicron (B.1.1.529) Variant. N Engl J Med, doi:10.1056/NEJMoa2119451 (2022).
8 Krause, P. R. et al. SARS-CoV-2 Variants and Vaccines. N Engl J Med 385, 179-186, doi:10.1056/NEJMsr2105280 (2021).
9 de Vries, R. D. et al. Intranasal fusion inhibitory lipopeptide prevents direct-contact SARS-CoV-2 transmission in ferrets. Science 371, 1379-1382, doi:10.1126/science.abf4896 (2021).
10 Chai, J. et al. Structural basis for SARS-CoV-2 envelope protein recognition of human cell junction protein PALS1. Nat Commun 12, 3433, doi:10.1038/s41467-021-23533-x (2021).
11 Castano-Rodriguez, C. et al. Role of Severe Acute Respiratory Syndrome Coronavirus Viroporins E, 3a, and 8a in Replication and Pathogenesis. mBio 9, doi:10.1128/mBio.02325-17 (2018).
12 Nieto-Torres, J. L. et al. Severe acute respiratory syndrome coronavirus envelope protein ion channel activity promotes virus fitness and pathogenesis. PLoS Pathog 10, e1004077, doi:10.1371/journal.ppat.1004077 (2014).
13 Nieva, J. L., Madan, V. & Carrasco, L. Viroporins: structure and biological functions. Nat Rev Microbiol 10, 563-574, doi:10.1038/nrmicro2820 (2012).
14 Nieto-Torres, J. L. et al. Subcellular location and topology of severe acute respiratory syndrome coronavirus envelope protein. Virology 415, 69-82, doi:10.1016/j.virol.2011.03.029 (2011).
15 DeDiego, M. L. et al. Severe acute respiratory syndrome coronavirus envelope protein regulates cell stress response and apoptosis. PLoS Pathog 7, e1002315, doi:10.1371/journal.ppat.1002315 (2011).
16 Mandala, V. S. et al. Structure and drug binding of the SARS-CoV-2 envelope protein transmembrane domain in lipid bilayers. Nat Struct Mol Biol 27, 1202-1208, doi:10.1038/s41594-020-00536-8 (2020).
17 Pervushin, K. et al. Structure and inhibition of the SARS coronavirus envelope protein ion channel. PLoS Pathog 5, e1000511, doi:10.1371/journal.ppat.1000511 (2009).
18 Zheng, M. et al. TLR2 senses the SARS-CoV-2 envelope protein to produce inflammatory cytokines. Nat Immunol 22, 829-838, doi:10.1038/s41590-021-00937-x (2021).
19 Cabrera-Garcia, D., Bekdash, R., Abbott, G. W., Yazawa, M. & Harrison, N. L. The envelope protein of SARS-CoV-2 increases intra-Golgi pH and forms a cation channel that is regulated by pH. J Physiol 599, 2851-2868, doi:10.1113/JP281037 (2021).
20 Boson, B. et al. The SARS-CoV-2 envelope and membrane proteins modulate maturation and retention of the spike protein, allowing assembly of virus-like particles. J Biol Chem 296, 100111, doi:10.1074/jbc.RA120.016175 (2021).
21 Duart, G. et al. SARS-CoV-2 envelope protein topology in eukaryotic membranes. Open Biol 10, 200209, doi:10.1098/rsob.200209 (2020).
22 Surya, W., Li, Y. & Torres, J. Structural model of the SARS coronavirus E channel in LMPG micelles. Biochim Biophys Acta Biomembr 1860, 1309-1317, doi:10.1016/j.bbamem.2018.02.017 (2018).
23 Patel, S. G. et al. Cell-penetrating peptide sequence and modification dependent uptake and subcellular distribution of green florescent protein in different cell lines. Sci Rep 9, 6298, doi:10.1038/s41598-019-42456-8 (2019).
24 Macian, F., Lopez-Rodriguez, C. & Rao, A. Partners in transcription: NFAT and AP-1. Oncogene 20, 2476-2489, doi:10.1038/sj.onc.1204386 (2001).
25 Morikawa, K. et al. Photoactivatable Cre recombinase 3.0 for in vivo mouse applications. Nat Commun 11, 2141, doi:10.1038/s41467-020-16030-0 (2020).
26 Ghosh, S. et al. beta-Coronaviruses Use Lysosomes for Egress Instead of the Biosynthetic Secretory Pathway. Cell 183, 1520-1535 e1514, doi:10.1016/j.cell.2020.10.039 (2020).
27 Leist, S. R. et al. A Mouse-Adapted SARS-CoV-2 Induces Acute Lung Injury and Mortality in Standard Laboratory Mice. Cell 183, 1070-1085 e1012, doi:10.1016/j.cell.2020.09.050 (2020).
28 Song, L., Park, S. E., Isseroff, Y., Morikawa, K. & Yazawa, M. Inhibition of CDK5 Alleviates the Cardiac Phenotypes in Timothy Syndrome. Stem Cell Reports 9, 50-57, doi:10.1016/j.stemcr.2017.05.028 (2017).
29 Navarrete-Perea, J., Yu, Q., Gygi, S. P. & Paulo, J. A. Streamlined Tandem Mass Tag (SL-TMT) Protocol: An Efficient Strategy for Quantitative (Phospho)proteome Profiling Using Tandem Mass Tag-Synchronous Precursor Selection-MS3. J Proteome Res 17, 2226-2236, doi:10.1021/acs.jproteome.8b00217 (2018).
30 Tyanova, S. et al. The Perseus computational platform for comprehensive analysis of (prote)omics data. Nat Methods 13, 731-740, doi:10.1038/nmeth.3901 (2016).
31 Bekdash, R. et al. GEM-IL: A highly responsive fluorescent lactate indicator. Cell Rep Methods 1, 100092, doi:10.1016/j.crmeth.2021.100092 (2021).

The scope of the present invention is not limited by what has been specifically shown and described hereinabove. Those skilled in the art will recognize that there are suitable alternatives to the depicted examples of materials, configurations, constructions and dimensions. Numerous references, including patents and various publications, are cited and discussed in the description of this invention. The citation and discussion of such references is provided merely to clarify the description of the present invention and is not an admission that any reference is prior art to the invention described herein. All references cited and discussed in this specification are incorporated herein by reference in their entirety. Variations, modifications and other implementations of what is described herein will occur to those of ordinary skill in the art without departing from the spirit and scope of the invention. While certain embodiments of the present invention have been shown and described, it will be obvious to those skilled in the art that changes and modifications may be made without departing from the spirit and scope of the invention. The matter set forth in the foregoing description is offered by way of illustration only and not as a limitation.

1. A fusion polypeptide, comprising:
(i) a cell-penetrating peptide; and
(ii) a coronavirus peptide comprising a fragment of an envelope (E) protein of a coronavirus.

2. The fusion polypeptide of claim 1, wherein the coronavirus peptide has about 10 to about 30 amino acid residues in length, and wherein the coronavirus peptide has an amino acid sequence at least 70% identical to a consecutive amino acid sequence within position 1 to position 40 of the envelope (E) protein of a coronavirus.

3. The fusion polypeptide of claim 2, wherein the coronavirus peptide has about 15 to about 20 amino acid residues in length, and wherein the coronavirus peptide has an amino acid sequence at least 80% identical to a consecutive amino acid sequence within position 1 to position 25 of the envelope (E) protein of a coronavirus.

4. The fusion polypeptide of claim 3, wherein the coronavirus peptide has about 18 amino acid residues in length, and wherein the coronavirus peptide has an amino acid sequence at least 80% identical to a consecutive amino acid sequence within position 1 to position 18 of the envelope (E) protein of a coronavirus.

5. The fusion polypeptide of any preceding claim, wherein the coronavirus peptide comprises at least one glutamic acid to aspartic acid mutation, and/or at least one aspartic acid to glutamic acid mutation.

6. The fusion polypeptide of any preceding claim, wherein the coronavirus peptide comprises an amino acid sequence at least 80% identical to the amino acid sequence set forth in SEQ ID NO: 1, SEQ ID NO: 13, SEQ ID NO: 14, SEQ ID NO: 17, SEQ ID NO: 19, SEQ ID NO: 22, or SEQ ID NO: 24.

7. The fusion polypeptide of any preceding claim, wherein the coronavirus peptide comprises an amino acid sequence set forth in SEQ ID NO: 2, SEQ ID NO: 15, SEQ ID NO: 16, SEQ ID NO: 18, SEQ ID NO: 20, SEQ ID NO: 21, SEQ ID NO: 23 or SEQ ID NO: 25.

8. The fusion polypeptide of any preceding claim, wherein the cell-penetrating peptide is TAT, 6-Arg or Penetratin.

9. The fusion polypeptide of any preceding claim, wherein the cell-penetrating peptide comprises an amino acid sequence at least 80% identical to the amino acid sequence set forth in SEQ ID NO: 4, SEQ ID NO: 5 or SEQ ID NO: 6.

10. The fusion polypeptide of any preceding claim, wherein the cell-penetrating peptide is directly linked to the coronavirus peptide.

11. The fusion polypeptide of any preceding claim, further comprising a linker connecting the cell-penetrating peptide and the coronavirus peptide.

12. The fusion polypeptide of claim 11, wherein the linker is a polyethylene glycol (PEG) linker.

13. The fusion polypeptide of claim 12, wherein the PEG linker is (PEG)3.

14. The fusion polypeptide of any preceding claim, wherein the fusion polypeptide is PEGylated.

15. The fusion polypeptide of any preceding claim, wherein the cell-penetrating peptide is located at the N-terminus of the fusion polypeptide.

16. The fusion polypeptide of any preceding claim, comprising an amino acid sequence at least 80% identical to the amino acid sequence set forth in SEQ ID NO: 7, SEQ ID NO: 9 or SEQ ID NO: 10.

17. The fusion polypeptide of any preceding claim, comprising an amino acid sequence set forth in SEQ ID NO: 3 or SEQ ID NO: 8.

18. A coronavirus peptide, having about 10 to about 30 amino acid residues in length, wherein the coronavirus peptide has an amino acid sequence at least 70% identical to a consecutive amino acid sequence within position 1 to position 40 of an envelope (E) protein of a coronavirus.

19. The coronavirus peptide of claim 18, having about 15 to about 20 amino acid residues in length, wherein the coronavirus peptide has an amino acid sequence at least 80% identical to a consecutive amino acid sequence within position 1 to position 25 of the envelope (E) protein of a coronavirus.

20. The coronavirus peptide of claim 19, having about 18 amino acid residues in length, wherein the coronavirus peptide has an amino acid sequence at least 80% identical to a consecutive amino acid sequence within position 1 to position 18 of the envelope (E) protein of a coronavirus.

21. The coronavirus peptide of any of claims 18-20, comprising an amino acid sequence at least 80% identical to the amino acid sequence set forth in SEQ ID NO: 1, SEQ ID NO: 13, SEQ ID NO: 14, SEQ ID NO: 17, SEQ ID NO: 19, SEQ ID NO: 22, SEQ ID NO: 24.

22. The coronavirus peptide of any of claims 18-21, comprising an amino acid sequence set forth in SEQ ID NO: 2, SEQ ID NO: 15, SEQ ID NO: 16, SEQ ID NO: 18, SEQ ID NO: 20, SEQ ID NO: 21, SEQ ID NO: 23 or SEQ ID NO: 25.

23. The fusion polypeptide or the coronavirus peptide of any preceding claim, wherein the coronavirus is a human coronavirus.

24. The fusion polypeptide or the coronavirus peptide of any preceding claim, wherein the coronavirus is SARS-CoV-2, SARS-CoV-1, MERS-CoV, HCoV-229E, HCoV-NL63, HCoV-OC43 or HCoV-HKU1.

25. A nucleic acid encoding the fusion polypeptide or the coronavirus peptide of claims 1-24.

26. A pharmaceutical composition, comprising the fusion polypeptide or the coronavirus peptide of any of claims 1-24 or the nucleic acid of claim 25.

27. A kit comprising the fusion polypeptide, the coronavirus peptide, or the pharmaceutical composition of any preceding claim.

28. A method of treating or preventing infection by a coronavirus in a subject, the method comprising administering the fusion polypeptide or the coronavirus peptide of any of claims 1-24 to the subject.

29. A method of treating or preventing infection by a coronavirus in a subject, the method comprising administering the pharmaceutical composition of claim 26 to the subject.

30. A method of treating or preventing infection by a coronavirus in a subject, the method comprising administering the nucleic acid of claim 25 to the subject.

31. The method of any of claims 28-30, wherein the coronavirus is a human coronavirus.

32. The method of any of claims 28-30, wherein the coronavirus is SARS-CoV-2, SARS-CoV-1, MERS-CoV, HCoV-229E, HCoV-NL63, HCoV-OC43 or HCoV-HKU1.
